(19) United States
(12) Patent Application Publication
(10) Pub. No.: US 2022/0073635 A1
(43) Pub. Date: Mar. 10, 2022

(54) NOVEL POLYPEPTIDES

(71) Applicant: ALLIGATOR BIOSCIENCE AB, Lund (SE)

(72) Inventors: Anna Sall, Lund (SE); Peter Ellmark, Lund (SE); Adnan Deronic, Lund (SE); Fredrika Carlsson, Lund (SE); Karin Hagerbrand, Lund (SE); Laura Von Schantz, Lund (SE)

(21) Appl. No.: 17/312,630

(22) PCT Filed: Dec. 17, 2019
(86) PCT No.: PCT/EP2019/085789

§ 371 Date: Jun. 10, 2021

(30) Foreign Application Priority Data
Dec. 17, 2018 (GB) 1820556.7
Apr. 17, 2019 (GB) 1905458.4
Jul. 12, 2019 (GB) 1910014.8

(51) Int. Cl. C07K 16/28; C07K 16/30; A61K 45/06; A61K 39/395; A61P 35/00

(52) U.S. Cl. CPC C07K 16/2878; C07K 16/30; A61K 45/06; A61K 2039/505; A61K 39/39558; A61P 35/00; A61K 39/3955

(57) ABSTRACT

The invention provides bispecific polypeptides comprising a first binding domain, designated B1, which is capable of binding specifically to CD40, and a second binding domain, designated B2, which is capable of specifically binding to a tumour cell-associated antigen. Also provided are pharmaceutical compositions of such bispecific polypeptides and uses of the same in medicine.

### FIELD OF INVENTION

The present invention relates to novel bispecific polypeptides, such as antibodies, and their use in the treatment of cancers.

### BACKGROUND

### Immunotherapy of Cancer

Cancer is a leading cause of premature deaths in the developed world. Immunotherapy of cancer aims to mount an effective immune response against tumour cells. This may be achieved by, for example, breaking tolerance against tumour antigen, augmenting anti-tumour immune responses, and stimulating local cytokine responses at the tumour site. The key effector cell of a long-lasting anti-tumour immune response is the activated tumour-specific effector T cell. Potent expansion of activated tumour-specific effector T cells can redirect the immune response towards the tumour. In this context, various immunosuppressive mechanisms induced by the tumour microenvironment suppress the activity of effector T cells. Several immunosuppressive mediators are expressed by the tumour cells. Such mediators inhibit T cell activation, either directly, or indirectly by inducing e.g. regulatory T cells (Treg) or myeloid-derived suppressor cells. Depleting, inhibiting, reverting or inactivating such regulatory cells may therefore provide anti-tumour effects and revert the immune suppression in the tumour microenvironment. Further, incomplete activation of effector T cells by, for example, dendritic cells (DC) can result in sub-optimally activated or anergic T cells, resulting in an inefficient anti-tumour response. In contrast, adequate induction by DC can generate a potent expansion of activated effector T cells, redirecting the immune response towards the tumour. In addition, natural killer (NK) cells play an important role in tumour immunology by attacking tumour cells with down-regulated human leukocyte antigen (HLA) expression and by inducing antibody dependent cellular cytotoxicity (ADCC). Stimulation of NK cells may thus also reduce tumour growth.

### Tumour Cell-Associated Antigens

Tumour cell-associated antigens (TAA) are cell surface proteins selectively expressed on tumour cells. The term tumour cell-associated indicates that TAA are not completely tumour-specific, but are rather over-expressed on the tumour. A vast number of TAA have been described and used in various therapeutic rationales, including monoclonal antibodies, T cell-redirecting therapies with TAA-CD3 bispecific antibodies, immunocytokines and antibody-drug conjugates. Some well-studied TAA include the EGFR family molecules (HER2, HER3 and EGFR/HER1), VEGFR, EpCAM, CEA, PSA, PSMA, EphA2, gp100, GD2, MUC1, CD20, CD19, CD22 and CD33, summarized in (Cheever et al., 2009, Clin Cancer Res).

5T4 (also designated trophoblast glycoprotein, TPBG, M6P1 and Waif1) is a well-defined TAA originally identified by Professor Peter Stern, University of Manchester (Hole and Stern, 1988, Br J Cancer). It is an oncofetal antigen expressed in a high proportion of patients in a variety of malignancies, including non-small cell lung, renal, pancreas, prostate, breast, colorectal, gastric, ovarian and cervix cancers as well as in acute lymphocytic leukemia, and has also been shown to be expressed in tumour-initiating cells (Castro et al., 2012, Leukemia; Damelin et al., 2011, Cancer Res; Elkord et al., 2009, Expert Rev Anticancer Ther; Southall et al., 1990, Br J Cancer).

5T4 expression is tumour-selective, with no or low expression in most normal tissues. In non-malignant tissue, 5T4 is mainly expressed in the placenta (trophoblast and amniotic epithelium) and at low levels in some specialised epithelia (Hole and Stern, 1988, Br J Cancer), as well as low at levels in other normal tissues (see US 2010/0021483). However, although low levels have been detected in some healthy tissue, the safety risk associated with this is considered low since expression levels in the tumour are considerably higher. This is supported by the fact that the phase III clinical programs, ANYARA and TroVax targeting 5T4 did not report severe 5T4-related toxicities.

Data from Stern et al. demonstrate that 5T4 regulates the functional activity of CXCR4 (Castro et al., 2012, Leukemia; Southgate et al., 2010, PLoS One). 5T4 binding antibodies or 5T4 knock-down resulted in inhibition of CXCR4-mediated cellular migration. The CXCR4 pathway is involved in tumour growth and metastasis. Therefore, targeting 5T4 in a CXCR4 inhibitory manner is likely to reduce tumour growth and/or spread.

EpCAM (Alternative names: BerEp4, CD326, CO-171A, 17-1A, EpCAM/Ep-AM, ESA, EGP, EGP-2, EGP34, EGP40, GA733-2, HEA125, KSA, KS1/4, MH99, MK-1, MOC31, TROP 1, VU-1D9, 323/A3) is overexpressed on malignant carcinomas (Patriarca et al., 2012, Cancer Treatment Reviews) (Yao et al. 2013, Int J Cancer) (Lund et al., 2014, mABs) (Schnell et al., 2013, Biochim Biophys Acta). EpCAM is a type I, transmembrane, 39-42 kDa glycoprotein that functions as a epithelial-specific intercellular adhesion molecule (Patriarca et al., 2012, Cancer Treatment Reviews).

EGFR is amplified and dysregulated on several cancer types. EGFR is expressed in different conformations which are functionally active, or inactive, and can be discriminated by specific antibodies. EGFR regulates cellular growth, apoptosis, migration, adhesion and differentiation (Yarden, 2001, Eur J Cancer; Yarden and Sliwkowski, 2001, Nat Rev Mol Cell Biol). Overexpression or continuous signalling through this receptor is common in carcinomas.

HER2, also known as CD340 (cluster of differentiation 340), proto-oncogene Neu, Erbb2 (rodent), or ERBB2, is amplified and dysregulated in many tumour types, in particular in breast cancer (Yarden, 2001, Eur J Cancer). Over-expression of this oncogene has been shown to play an important role in the development and progression of cancer.

### CD40

CD40 is a cell-surface expressed glycoprotein that belongs to the tumour necrosis factor receptor (TNFR) superfamily and plays a central role in the immune system. It is expressed on a variety of immune cells, such as B cells, DC, monocytes and macrophages, but also on other normal tissues including epithelial cells, endothelial cells and fibroblasts, as well as several tumour types, e.g. on B cell lymphomas and carcinomas. Activation of CD40 on DC results in an anti-tumour immune response via tumour-specific T effector cells. CD40 agonists trigger effective anti-tumour responses in pre-clinical models. These responses are mediated via two distinct mechanisms: (i) tumour-specific immune activation, and (ii) direct tumoricidal effects, via e.g. apoptosis, antibody-dependent cellular cytotoxicity (ADCC) and complement-dependent cytotoxicity (CDC). The anti-tumour immune effect, which is independent on the CD40 status of the tumour, is associated with activation of tumour-specific cytotoxic T lymphocytes (CTL), and possibly macrophages in certain tumour types. The direct tumoricidal effects on the other hand, are highly dependent on the CD40 expression of the tumour, and are thought to augment the anti-tumour immune response through the release of tumour antigens.

Pre-clinical studies have demonstrated proof of concept for agonistic anti-CD40 antibody treatment of several cancer types, including lymphomas, melanoma, hepatoma, osteosarcoma, renal cell carcinoma, breast cancer and bladder cancer. In addition, humanized or human anti-CD40 antibodies have been evaluated in a number of pre-clinical models and consistently demonstrated anti-tumour effects. Notably, SGN-40, a humanized CD40 monoclonal antibody with partial agonistic effects was evaluated using B cell lymphoma models (Raji and Ramos) in severe combined immunodeficiency (SCID) mice, and demonstrated effects on tumour growth and survival with complete response in approximately 50% of treated mice. CP-870,893, a human agonistic anti-CD40 antibody, showed anti-tumour effects against B cell lymphoma, breast, colon, prostate, and pancreatic cancer in SCID mice. Efficacy was observed in CD40 positive as well as in CD40 negative tumours, thus demonstrating the ability of CP-870,893 to enhance anti-tumour immunity.

Despite progress in the development of immunotherapies for the treatment of various cancers over the last decade, there remains a need for new and efficacious agents.

Accordingly, the present invention seeks to provide improved polypeptide-based therapies for the treatment of cancer.

### SUMMARY OF INVENTION

A first aspect of the invention provides a bispecific polypeptide comprising a first binding domain, designated B1, which is capable of binding specifically to CD40, and a second binding domain, designated B2, which is capable of specifically binding to a tumour cell-associated antigen.

Such bispecific compounds comprising one tumor-targeting moiety, e.g. an EpCAM binder, and one immune-activating moiety, e.g. a CD40 agonist, can be used to establish a highly effective and safe cancer immunotherapy.

Various types of tumour-localizing immunotherapeutic molecules, such as immunocytokines and bispecific antibodies have shown beneficial immune activation and inhibition of tumor growth in preclinical studies as well as in the clinic (reviewed in Kiefer and Neri, 2016).

The clinical progress with immunocytokines has so far not been impressive and the side effects still remain since the tumor-binding entity only confers limited tumor localization, with the bulk of the immunocytokine ending up in other compartments. Bispecific antibodies that restrict the activity to the tumor as described in this invention would provide a clear advantage over immunocytokines since they are inactive in the absence of tumors.

To avoid affecting part of the immune system not relevant for inducing tumour immunity and avoid systemic toxicity by CD40-activating agents, yet obtain high efficacy in the tumour area, the designs of the molecular formats of CD40 agonists may be optimised. For example, a good efficacy/safety profile can be obtained by a CD40-TAA bispecific antibody that requires crosslinking by binding to the TAA for CD40 activation to occur. Thus, CD40-expressing cells such as dendritic cells, residing in the tumour tissue, will preferentially be activated, whereas CD40-expressing cells in other tissues, where the expression of TAA is low or absent, will not. This would allow focused activation of CD40-expressing cells specifically in the tumour tissue, while limiting toxicity induced by generalised CD40 activation.

### Structure of the Bispecific Polypeptide

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The term “bispecific” as used herein means the polypeptide is capable of specifically binding at least two target entities.

In one preferred embodiment, the polypeptide is a bispecific antibody (numerous examples of which are described in detail below).

Thus, the first and/or second binding domains may be selected from the group consisting of antibodies and antigen-binding fragments thereof.

By “an antibody or an antigen-binding fragment thereof” we include substantially intact antibody molecules, as well as chimeric antibodies, humanised antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include Fv fragments (e.g. single chain Fv and disulphide-bonded Fv), Fab-like fragments (e.g. Fab fragments, Fab′ fragments and F(ab)2 fragments), single variable domains (e.g. VH and VL domains) and single domain antibodies (dAbs, including single and dual formats [i.e. dAb-linker-dAb], and nanobodies). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

In one embodiment, the antigen-binding fragment is selected from the group consisting of: Fv fragments (such as a single chain Fv fragment, or a disulphide-bonded Fv fragment), Fab-like fragments (such as a Fab fragment; a Fab′ fragment or a F(ab)₂ fragment) and single domain antibodies.

The phrase “an antibody or an antigen-binding fragment thereof” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

Also included within the scope of the invention are chimeric T cell receptors (also known as chimeric immunoreceptors, and chimeric antigen receptors or CARs) (see Pule et al., 2003, the disclosures of which are incorporated herein by reference). These are engineered receptors, which graft an arbitrary specificity onto an immune effector cell. Typically, CARs are used to graft the specificity of a monoclonal antibody onto a T cell; with transfer of their coding sequence facilitated by retroviral vectors. The most common form of such molecules is fusions comprising a single-chain variable fragment (scFv) derived from a monoclonal antibody fused to CD3-zeta transmembrane and endodomain. When T cells express this fusion molecule, they recognize and kill target cells that express the transferred monoclonal antibody specificity.

Persons skilled in the art will further appreciate that the invention also encompasses modified versions of antibodies and antigen-binding fragments thereof, whether existing now or in the future, e.g. modified by the covalent attachment of polyethylene glycol or another suitable polymer (see below).

Methods of generating antibodies and antibody fragments are well known in the art. For example, antibodies may be generated via any one of several methods which employ induction of in vivo production of antibody molecules, screening of immunoglobulin libraries (Orlandi. et al, 1989; Winter et al., 1991, the disclosures of which are incorporated herein by reference) or generation of monoclonal antibody molecules by cell lines in culture. These include, but are not limited to, the hybridoma technique, the human B cell hybridoma technique, and the Epstein-Barr virus (EBV)-hybridoma technique (Kohler et al., 1975, Kozbor et al., 1985; Cote et al., 1983; Cole et al., 1984, the disclosures of which are incorporated herein by reference).

Suitable methods for the production of monoclonal antibodies are also disclosed in “Monoclonal Antibodies: A manual of techniques”, H Zola (CRC Press, 1988, the disclosures of which are incorporated herein by reference) and in “Monoclonal Hybridoma Antibodies: Techniques and Applications”, J G R Hurrell (CRC Press, 1982, the disclosures of which are incorporated herein by reference).

Likewise, antibody fragments can be obtained using methods well known in the art (see, for example, Harlow & Lane, 1988, “Antibodies: A Laboratory Manual”, Cold Spring Harbor Laboratory, New York, the disclosures of which are incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy or diagnostics, human or humanised antibodies are preferably used. Humanised forms of non-human (e.g. murine) antibodies are genetically engineered chimaeric antibodies or antibody fragments having preferably minimal-portions derived from non-human antibodies. Humanised antibodies include antibodies in which complementary determining regions of a human antibody (recipient antibody) are replaced by residues from a complementary determining region of a non-human species (donor antibody) such as mouse, rat or rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986, Riechmann et al., 1988, Presta, 1992, the disclosures of which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Reichmann et al., 1988, Verhoeyen et al., 1988, U.S. Pat. No. 4,816,567, the disclosures of which are incorporated herein by reference) by substituting human complementarity determining regions with corresponding rodent complementarity determining regions. Accordingly, such humanised antibodies are chimaeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, Marks et al., 1991, Cole et al., 1985, Boerner et al., 1991, the disclosures of which are incorporated herein by reference).

It will be appreciated by persons skilled in the art that the bispecific polypeptides, e.g. antibodies, of the present invention may be of any suitable structural format.

Thus, in exemplary embodiments of the bispecific antibodies of the invention:

(a) binding domain B1 and/or binding domain B2 is an intact IgG antibody (or, together, form an intact IgG antibody);
(b) binding domain B1 and/or binding domain B2 is an Fv fragment (e.g. an scFv);
(c) binding domain B1 and/or binding domain B2 is a Fab fragment; and/or
(d) binding domain B1 and/or binding domain B2 is a single domain antibody (e.g. domain antibodies and nanobodies).

It will be appreciated by persons skilled in the art that the bispecific antibody may comprise a human Fc region, or a variant of a said region, where the region is an IgG1, IgG2, IgG3 or IgG4 region, preferably an IgG1 or IgG4 region.

Engineering the Fc region of a therapeutic monoclonal antibody or Fc fusion protein allows the generation of molecules that are better suited to the pharmacology activity required of them (Strohl, 2009, the disclosures of which are incorporated herein by reference).

### (a) Engineered Fc Regions for Increased Half-Life

One approach to improve the efficacy of a therapeutic antibody is to increase its serum persistence, thereby allowing higher circulating levels, less frequent administration and reduced doses.

The half-life of an IgG depends on its pH-dependent binding to the neonatal receptor FcRn. FcRn, which is expressed on the surface of endothelial cells, binds the IgG in a pH-dependent manner and protects it from degradation.

Some antibodies that selectively bind the FcRn at pH 6.0, but not pH 7.4, exhibit a higher (to put another way longer) half-life in a variety of animal models. Additionally, some antibodies that bind the FcRn with a higher affinity at pH 6.0, but with a remained low affinity at pH 7.4 exhibit a longer half-life.

Several mutations located at the interface between the CH2 and CH3 domains, such as T250Q/M428L (Hinton et al., 2004, the disclosures of which are incorporated herein by reference) and M252Y/S254T/T256E+H433K/N434F (Vaccaro et al., 2005, the disclosures of which are incorporated herein by reference), have been shown to increase the binding affinity to FcRn and the half-life of IgG1 in vivo.

### (b) Engineered Fc Regions for Altered Effector Function

To ensure lack of CD40 activation in the absence of the tumour antigen, the Fc portion of the bispecific antibody should bind with no or very low affinity to FcγR, since FcγR-mediated crosslinking of a CD40 antibody may induce activation. By “very low affinity” we include that the Fc portion exhibits at least 10 times reduced affinity to FcγRI, FcγRII and III compared to wild-type IgG1, as determined by the concentration where half maximal binding is achieved in flow cytometric analysis of FcγR expressing cells (Hezareh et al., 2001) or by FcγR ELISA (Shields et al., 2001).

Another factor to take into account is that engagement of FcγRs may also induce antibody-dependent cellular cytotoxicity (ADCC), antibody-dependent cellular phagocytosis (ADCP) and complement-dependent cytotoxicity (CDC) of cells coated with antibodies. In one embodiment, to enhance tumour-dependent CD40 activation as well as to avoid depletion of CD40-expressing cells, the isotype of a CD40-TAA bispecific antibody should preferably be silent.

The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions (Bruhns et al., 2009, the disclosures of which are incorporated herein by reference). IgG1 molecules have the highest affinity and capacity to induce effector functions, whereas IgG2, IgG3 and IgG4 are less effective (Bruhns, 2012; Hogarth and Pietersz, 2012; Stewart et al., 2014) (Wang et al. 2015; Vidarson et al. 2014). In addition, certain mutations in the Fc region of IgG1 dramatically reduce FcγR affinity and effector function while retaining neonatal FcR (FcRn) interaction (Ju and Jung, 2014; Leabman et al., 2013; Oganesyan et al., 2008; Sazinsky et al., 2008).

The most widely used IgG1 mutants are N297A alone or in combination with D265A, as well as mutations at positions L234 and L235, including the so-called “LALA” double mutant L234A/L235A. Another position described to further silence IgG1 by mutation is P329 (see US 2012/0251531).

Thus, choosing a mutated IgG1 format with low effector function but retained binding to FcRn may result in a bispecific antibody with TAA-dependent activation of CD40, and exhibiting a favorable efficacy/safety profile and good PK properties.

Advantageously, the polypeptide is incapable of inducing antibody-dependent cell cytotoxicity (ADCC), antibody-dependent cellular phagocytosis (ADCP), and/or complement-dependent cytotoxicity (CDC). By “incapable” we include that the ability of the polypeptide to induce ADCC, etc., is at least 10-fold lower than compared to wild-type IgG1 as shown by e.g. monocyte-dependent ADCC or CDC assays described by Hezareh et al. 2001.

In one embodiment, the Fc region may be a variant of a human IgG1 Fc region comprising a mutation at one or more of the following positions:

L234, L235, P239, D265, N297 and/or P329.

Advantageously, alanine may be present at the mutated position(s).

Optionally, the IgG1 variant may be a variant of a human IgG1 Fc region comprising mutations L234A and L235A (i.e. the LALA double mutant; see SEQ ID NO: 161).

It will be appreciated by persons skilled in the art that the bispecific polypeptides of the invention may be of several different structural formats (for example, see Chan & Carter, 2016, the disclosures of which are incorporated herein by reference).

In exemplary embodiments, the bispecific antibody is selected from the groups consisting of:

(a) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
(b) monovalent bispecific antibodies, such as a DuoBody® (Genmab AS, Copenhagen, Denmark) or ‘knob-in-hole’ bispecific antibody (for example, an scFv-KIH, scFv-KIH^{r}, a BiTE-KIH or a BiTE-KIH^{r} (see Xu et al., 2015, mAbs 7(1):231-242));
(c) scFv₂-Fc bispecific antibodies (such as ADAPTIR™ bispecific antibodies from Emergent Biosolutions Inc);
(d) BiTE/scFv₂ bispecific antibodies;
(e) DVD-Ig bispecific antibodies;
(f) DART-based bispecific antibodies (for example, DART₂-Fc or DART);
(g) DNL-Fab₃ bispecific antibodies; and
(h) scFv-HSA-scFv bispecific antibodies.

For example, the bispecific antibody may be an IgG-scFv antibody. The IgG-scFv antibody may be in either VH-VL or VL-VH orientation. In one embodiment, the scFv may be stabilised by a S—S bridge between VH and VL.

In one embodiment, binding domain B1 and binding domain B2 are fused directly to each other.

In an alternative embodiment, binding domain B1 and binding domain B2 are joined via a polypeptide linker. For example, a polypeptide linker may be a short linker peptide between about 10 to about 25 amino acids. The linker is usually rich in glycine for flexibility, as well as serine or threonine for solubility, and can either connect the N-terminus of the VH with the C-terminus of the VL, or vice versa.

Thus, the linker may be selected from the group consisting of the amino acid sequence SGGGGSGGGGS (SEQ ID NO: 162), SGGGGSGGGGSAP (SEQ ID NO: 163), NFSQP (SEQ ID NO: 164), KRTVA (SEQ ID NO: 165), GGGSGGGG (SEQ ID NO: 166), GGGGSGGGGS, (SEQ ID NO: 167), GGGGSGGGGSGGGGS (SEQ ID NO: 168), GSTSGSGKPGSGEGSTKG (SEQ ID NO: 169) (Whitlow et al. 1993) THTCPPCPEPKSSDK (SEQ ID NO: 170), GGGS (SEQ ID NO: 171), EAAKEAAKGGGGS (SEQ ID NO: 172), EAAKEAAK (SEQ ID NO: 173), or (SG)m, where m=1 to 7.

In a preferred embodiment, the linker may be selected from the group consisting of: SEQ ID NO: 166, SEQ ID NO: 168 and SEQ ID NO: 169.

The term “amino acid” as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as “alanine” or “Ala” or “A”, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the polypeptides as defined herein comprise or consist of L-amino acids.

It will be appreciated by persons skilled in the art that the polypeptides of the invention may comprise or consist of one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. The term ‘peptidomimetic’ refers to a compound that mimics the conformation and desirable features of a particular peptide as a therapeutic agent.

For example, the said polypeptide includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997), which is incorporated herein by reference. This approach involves making pseudo-peptides containing changes involving the backbone, and not the orientation of side chains. Retro-inverse peptides, which contain NH—CO bonds instead of CO—NH peptide bonds, are much more resistant to proteolysis. Alternatively, the said polypeptide may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will also be appreciated that the said polypeptide may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978 and Thursell et al., 1983, which are incorporated herein by reference.

In one embodiment of the invention, one of binding domain B1 or binding domain B2 is an immunoglobulin molecule, and one of binding domain B1 or binding domain B2 is a Fab fragment, wherein the Fab fragment is fused to the C terminus of the heavy chain of the immunoglobulin via the light chain of the Fab fragment.

For example, the polypeptide may have a format as shown in FIG. 2. Such a format is referred to as the RUBY™ format (as described in pending UK patent application 1820556.7).

The bispecific polypeptide may comprise one or more mutations to promote association of the heavy chain polypeptide of the immunoglobulin with the light chain polypeptide of the immunoglobulin and/or to promote association of the heavy chain polypeptide of the Fab with the light chain polypeptide of the Fab.

In one embodiment the one or more mutations prevent the formation of aggregates and a Fab by-product.

It will be appreciated by persons skilled in the art, that in one embodiment the mutations may prevent the formation of aggregates and/or a Fab by-product by generating steric hindrance and/or incompatibility between charges.

By “steric hindrance” we mean the slowing of a reaction due to steric bulk, i.e. the size of an amino acid molecule prevents association of two protein surfaces that may otherwise occur if a smaller amino acid is present.

By “incompatibility between charges” we mean that an unwanted product will not form as the charges are incompatible and prevent the product from forming, e.g. there may be two negatively charged portions which repel and prevent an unwanted product from forming.

As described above, said mutations limit the formation of a Fab by-product and/or aggregates by, for example, creating surfaces that limit the formation of aggregates or by-product Fab fragments. In one embodiment, the mutations prevent formation of a Fab by-product by generating steric hindrance and/or incompatibility between charges (leading to charge incompatibility of wrong chains). The mutations may also promote interactions between correct chains (i.e. between the first heavy chain polypeptide and the first light chain polypeptide, and/or between the second heavy chain polypeptide and the second light chain polypeptide) by, for example, creating salt or disulphide bridges.

Thus, the mutations may favour formation of the bispecific polypeptide.

In one embodiment, the percentage of aggregates formed during manufacturing is less than or equal to 25%. Optionally the percentage of aggregates is less than or equal to 20%, 17.5%, 15%, 13.5% or 10%. Preferably the percentage of aggregates is less than 10%. Optionally these measurements are carried out when the chains of the bispecific polypeptide are transfected at equal ratios, e.g. at a ratio of 1:1:1 when 3 chains are used during production.

Alternatively, the chain transfection ratio may be optimised. Optionally the % of aggregates when the chain transfection ratio is optimised may be less than or equal to 3.5%, 3%, 2.5% or 2%.

In one embodiment, the bispecific polypeptide comprises one or more mutation pairs each comprising two functionally compatible mutations.

By “functionally compatible mutations” we mean the mutations have complementary functions, e.g. one mutation of the pair (in one chain) may be a mutation that forms a positively charged region, and the other mutation (in another chain) forms a negatively charged region. Together these mutations act in a functionally compatible way promoting association of the respective chains.

In one embodiment, the bispecific polypeptide comprises one or more mutation pairs in one or more of the following region groups:

(a) the CH1 and CKappa or CLambda region of the immunoglobulin; and/or
(b) the CH1 and CKappa or CLambda region of the Fab; and/or
(c) the VL and VH regions of the immunoglobulin; and/or
(d) the VL and VH regions of the Fab.

Thus, in one embodiment, the mutation pairs are in the CH1 and CKappa or CLambda regions of the Fab and/or the immunoglobulin, and the mutation pairs are selected from:

(a) cavity and protruding surface mutations (i.e. steric mutations); and/or
(b) hydrophobic swap mutations; and/or
(c) charged mutations (i.e. salt mutations); and/or
(d) mutations resulting in the formation of a disulphide bridge.

The mutation pairs may alternatively or additionally be in the VH and VL regions of the Fab and/or the immunoglobulin, the mutation pairs in the VH and VL regions are selected from:

(a) charged mutations (i.e. salt mutations); and/or
(b) double charged mutations; and/or
(c) mutations resulting in the formation of a disulphide bridge.

In one embodiment of the invention the mutations are at positions selected from the group consisting of:

(a) one or more of the following positions in the CH1 domain: H168, F170, L145, S183 and T187 (according to EU numbering); and/or
(b) a position selected from the one or more of the following position ranges in the CKappa or CLambda domain: position 132 to 138, position 173 to 179, position 130 to 136, position 111 to 117 and position 134 to 140 (according to EU numbering); and/or
(c) a position selected from one or more of the following position ranges in the VL: position 41 to 47, position 117 to 123 and position 46 to 52 (according to IMGT numbering); and/or
(d) a position selected from one or more of the following position ranges in the VH: position 41 to 47, position 46 to 52 and position 117 to 123 (according to IMGT numbering).

In one embodiment of the invention the mutations are at positions selected from the group consisting of:

(a) one or more of the following positions in the CH1 domain: H168, F170, L145, 5183 and T187 (according to EU numbering); and/or
(b) a position selected from the one or more of the following position ranges in the CKappa or CLambda domain: position 132 to 138, position 173 to 179, position 130 to 136, position 111 to 117 and position 134 to 140 (according to Kabat numbering); and/or
(c) a position selected from one or more of the following position ranges in the VL: position 41 to 47, position 117 to 123 and position 46 to 52 (according to IMGT numbering); and/or
(d) a position selected from one or more of the following position ranges in the VH: position 41 to 47, position 46 to 52 and position 117 to 123 (according to IMGT numbering).

One mutation in each of the ranges given above will be the relevant functional mutation as it will be a position that makes contact with the amino acid in the corresponding domain/chain, and is therefore the relevant interface between chains.

It will therefore be appreciated by persons skilled in the art that mutations in the position ranges given above are suitable, as the relevant functional feature is whether the position contacts a corresponding position on the other chain, i.e. a position in the VH chain that contacts a corresponding position in a VL chain is the relevant position, or a position in a CLambda that contacts a position in a CH1 chain is the relevant position.

In one embodiment the mutations are selected from the group consisting of:

| | |
|---|---|
| VH | X44R/E/D/K, X49C, X120K |
| VL | X44R/E/D/K, X49D X120C |
| CH1 | H168A/G, F170G/A, L145Q, S183V, T187E/D, |
| CKappa/CLambda | S/T114A, V133T, L135Y/W, N/S137K/R/H, |
| | S176W/V/Y |

*numbering according to IMGT system for VH/VL domains and according to EU numbering system for constant domains
*X refers to any amino acid

In one embodiment the mutations are selected from the group consisting of:

| | |
|---|---|
| VH | X44R/E/D/K, X49C, X120K |
| VL | X44R/E/D/K, X49D X120C |
| CH1 | H168A/G, F170G/A, L145Q, S183V, T187E/D, |
| CKappa/CLambda | S/T114A, V133T, L135Y/W, N/S137K/R/H, |
| | S176W/V/Y |

*numbering according to IMGT system for VH/VL domains and according to Kabat numbering system for constant domains
*X refers to any amino acid

In one embodiment of the invention, the mutations are at positions selected from the group consisting of:

(a) one or more of the following positions in the CH1 domain: H168, F170, L145, S183 and T187 (according to EU numbering); and/or
(b) one or more of the following positions in the CKappa or CLambda domain: L135, S176, V133, S114 and N137 (according to EU numbering); and/or
(c) one or more of the following positions in the VL: Q44, Q120 and A49 (according to IMGT numbering); and/or
(d) one or more of the following positions in the VH: Q44, G49 and Q120 (according to IMGT numbering).

In one embodiment of the invention, the mutations are at positions selected from the group consisting of:

(a) one or more of the following positions in the CH1 domain: H168, F170, L145, S183 and T187 (according to EU numbering); and/or
(b) one or more of the following positions in the CKappa or CLambda domain: L135, S176, V133, S114 and N137 (according to Kabat numbering); and/or
(c) one or more of the following positions in the VL: Q44, Q120 and A49 (according to IMGT numbering); and/or
(d) one or more of the following positions in the VH: Q44, G49 and Q120 (according to IMGT numbering).

For example, the mutations may be selected from the group consisting of:

(a) one or more of the following mutations in the CH1 domain: H168A, F170G, L145Q, 183V and T187E (according to EU numbering); and/or
(b) one or more of the following mutations in the CKappa or CLambda domain: L135Y, S176W, V133T, S176V, S114A and N137K (according to EU numbering); and/or
(c) one or more of the following mutations in the VL: Q44R, Q44E, Q120C, Q44D and A49D (according to IMGT numbering); and/or
(d) one or more of the following mutations in the VH: Q44E, Q44R, G49C, Q44K and Q120K (according to IMGT numbering).

For example, the mutations may be selected from the group consisting of:

(a) one or more of the following mutations in the CH1 domain: H168A, F170G, L145Q, S183V and T187E (according to EU numbering); and/or
(b) one or more of the following mutations in the CKappa or CLambda domain: L135Y, S176W, V133T, S176V, S114A and N137K (according to Kabat numbering); and/or
(c) one or more of the following mutations in the VL: Q44R, Q44E, Q120C, Q44D and A49D (according to IMGT numbering); and/or
(d) one or more of the following mutations in the VH: Q44E, Q44R, G49C, Q44K and Q120K (according to IMGT numbering).

In one embodiment, the one or more Fab fragment(s) is linked to the C-terminal end of the immunoglobulin via a linker.

In one embodiment of the first aspect of the invention, the bispecific polypeptide is tetravalent, capable of binding bivalently to each of the two antigens.

In one embodiment, the bispecific polypeptide comprises an immunoglobulin arranged as an antibody with two arms and therefore two binding sites for the first antigen, and two of the Fab fragments, each providing a binding site for the second antigen. Thus, there are two binding sites for the first antigen and two binding sites for the second antigen.

In one embodiment, binding domain B1 is an immunoglobulin and binding domain B2 is a Fab.

Thus, in one embodiment the first antigen (targeted by the immunoglobulin) may be CD40 and the second antigen (targeted by the Fab) may be EpCAM.

In one embodiment, the bispecific polypeptide may modulate the activity of a target immune system cell, wherein said modulation is an increase or decrease in the activity of said cell. Such cells include T cells, dendritic cells and natural killer cells.

The immune system cell (for example, the target immune cell) is typically a dendritic cell. For example, the bispecific polypeptide may be capable of inducing activation of dendritic cells, which are then capable of internalising tumour associated debris or extracellular vesicles containing tumour-cell associated antigens and tumour neoantigens.

For example, the polypeptide may be capable of inducing:

(a) tumour-specific immune activation; and/or
(b) activation of dendritic cells; and/or
(c) internalisation of associated tumour debris and/or extracellular vesicles containing tumour cell-associated antigens as well as tumour neoantigens; and/or
(d) cross-presentation of peptides derived from internalised tumour antigens on MHC; and/or
(e) priming and activation of effector T cells; and/or
(f) direct tumoricidal effects, selected from the list consisting of: apoptosis, antibody-dependent cellular cytotoxicity (ADCC) and complement-dependent cytotoxicity (CDC).

It will be appreciated by persons skilled in the art, that said activation of dendritic cells may be an increase in the expression of the co-stimulatory molecules CD40, CD80 or CD86, or increased IL-12 production. Alternatively, activation of dendritic cells can be determined by the increased ability to cross-present antigens, e.g. tumour neoantigens, on MHC class I or II to T cells, generating an enhanced activation of T cells recognizing said antigen, by the antigen-presenting cell.

In one embodiment, the bispecific antibody induces an increase in the uptake of tumour debris or tumour extracellular vesicles by an antigen-presenting cell, such as a dendritic cell. It will be appreciated by persons skilled in the art, that said increase in uptake may be measured by the co-localization or internalization of the tumour debris or tumour extracellular vesicles by the antigen-presenting cell.

The increased uptake of tumour debris or tumour extracellular vesicles by the antigen-presenting cells would subsequently result in a broader T cell repertoire and, thus, more effective T cell-mediated tumour eradication. Methods for determining the expansion of tumour-antigen specific T cells are well known and include, for example, the use of MHC-peptide multimers, e.g. tetramers or pentamers. Such expansion may be measured by inoculating mice with tumours expressing a specific tumour antigen or tumours transfected with a tumour model antigen (e.g. ovalbumin), alternatively by inoculating mice with the same cells that have been heat shocked to induce necrosis, followed by measuring the expansion of tumour antigen-specific T cells by use of various MHC-tumour (model) antigen peptide tetramers or pentamers by flow cytometry-based methods.

The polypeptide or binding domains of the invention can also be characterised and defined by their binding abilities. Standard assays to evaluate the binding ability of ligands towards targets are well known in the art, including for example, ELISA, Western blot, RIA, and flow cytometry analysis. The binding kinetics (e.g., binding affinity) of the polypeptide can also be assessed by standard assays known in the art, such as by surface plasmon resonance analysis or bio-layer interferometry.

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods, and can be computed even for complex mixtures by methods such as those, for example, set forth in Caceci et al., 1984 (the disclosures of which are incorporated herein by reference). For example, the Kd may be established using a double-filter nitrocellulose filter binding assay such as that disclosed by Wong & Lohman, 1993. Other standard assays to evaluate the binding ability of ligands such as antibodies towards targets are known in the art, including for example, ELISA, Western blot, RIA, and flow cytometry analysis. The binding kinetics (e.g., binding affinity) of the polypeptide also can be assessed by standard assays known in the art, such as by surface plasmon resonance (by use of e.g. Biacore™ system analysis) or by bio-layer interferometry (by use of e.g. Octet® system analysis).

A competitive binding assay can be conducted in which the binding of the polypeptide to the target is compared to the binding of the target by another, known ligand of that target, such as another polypeptide. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 of polypeptides are set out in the Examples.

A polypeptide of the invention is preferably capable of binding to its target with an affinity that is at least two-fold, 10-fold, 50-fold, 100-fold or greater than its affinity for binding to another non-target molecule.

### CD40 Binding Domains

The bispecific polypeptides of the invention comprise a binding domain (B1) which is capable of specifically binding to CD40.

Binding domain B1 specifically binds to CD40, i.e. it binds to CD40 but does not bind, or binds at a lower affinity, to other molecules. The term CD40, as used herein, typically refers to human CD40. The sequence of human CD40 is set out in GenBank: X60592.1. Binding domain B1 may have some binding affinity for CD40 from other mammals, such as CD40 from a non-human primate (for example Macaca fascicularis (cynomolgus monkey), Macaca mulatta). Binding domain B1 preferably does not bind to murine CD40 and/or does not bind to other human TNFR superfamily members, for example human CD137 or OX40.

Advantageously, binding domain B1 binds to human CD40 with a K_{D} of less than 100×10⁻⁹M or less than 50×10⁻⁹M or less than 25×10⁻⁹M, preferably less than 10, 9, 8, 7, or 6×10⁻⁹M, more preferably less than 5, 4, 3, 2, or 1×10⁻⁹M, most preferably less than 9×10⁻¹⁰M.

For example, binding domain B1 preferably does not bind to murine CD40 or any other TNFR superfamily member, such as CD137 or OX40. Therefore, typically, the Kd for the binding domain with respect to human CD40 will be 2-fold, preferably 5-fold, more preferably 10-fold less than Kd with respect to the other, non-target molecules, such as murine CD40, other TNFR superfamily members, or any other unrelated material or accompanying material in the environment. More preferably, the Kd will be 50-fold less, even more preferably 100-fold less, and yet more preferably 200-fold less.

Binding domain B1 is preferably capable of binding to its target with an affinity that is at least two-fold, 10-fold, 50-fold, 100-fold or greater than its affinity for binding to another non-target molecule.

In summary therefore, binding domain B1 preferably exhibits at least one of the following functional characteristics:
a) binding to human CD40 with a K_{D} value which is less than 100×10⁻⁹M, more preferably less than 10×10⁻⁹M;
b) does not bind to murine CD40;
c) does not bind to other human TNFR superfamily members, for example human CD137 or OX40.

In one embodiment, binding domain B1 comprises one or more light chain CDR sequences selected from those in Table C(2), and/or one or more heavy chain CDR sequences selected from Table C(1). Thus binding domain B1 may comprise one or more CDR sequences selected from the groups consisting of:

(a) CD40 heavy chain CDRs, SEQ ID NOs: 73 to 89; and/or
(b) CD40 light chain CDRs, SEQ ID NOs: 90 to 104.

In one embodiment binding domain B1 comprises one, two or three light chain CDR sequences from a particular row for an individual antibody reference in Table C(2), and/or one, two or three heavy chain CDR sequences from the corresponding row for the antibody with the same reference in Table C(1). For example, binding domain B1 might comprise one or more of the light chain CDR sequences for 1132 (SEQ ID NOs: 90, 91 and 92) and one or more of the heavy chain CDR sequences for 1132 (SEQ ID NOs: 73, 74 and 75).

Preferred CD40 binding domains may comprise at least a heavy chain CDR3 as defined in any individual row of Table C(1) and/or a light chain CDR3 as defined in in any individual row of Table C(2).

Accordingly, in one embodiment binding domain B1 comprises all six CDR sequences for a given antibody (VH/VL) reference, for example binding domain B1 might comprise all six CDR sequences of antibody 1132.

In one embodiment, binding domain B1 comprises a VH and/or a VL amino acid sequence as given in Table A. In one embodiment, binding domain B1 comprises a VH and VL amino acid sequence as given in Table A for a particular antibody reference. For example, binding domain B1 may comprise the VH sequence of 1132 (SEQ ID NO: 3) and/or the VL sequence of 1132 (SEQ ID NO: 1).

In one embodiment the CD40 binding domain of B1 is selected from: 1132; 1150, 1140, 1107, ADC-1013, APX005 and 21.4.1.

Thus, the CDR or VH and VL sequences of binding domain B1 might be selected from antibodies from the group consisting of:

(a) 1132 (heavy chain CDRs: SEQ ID NOs: 73, 74 and 75; light chain CDRs: SEQ ID NOs: 90, 91, and 92; VL: SEQ ID NO: 1; VH: SEQ ID NO: 3)
(b) 1150 (heavy chain CDRs: SEQ ID NOs: 73, 76 and 77; light chain CDRs: SEQ ID NOs: 90, 91, and 93; VL: SEQ ID NO: 5; VH: SEQ ID NO: 7)
(c) 1140 (heavy chain CDRs: SEQ ID NOs: 73, 78 and 79; light chain CDRs: SEQ ID NOs: 90, 91, and 94; VL: SEQ ID NO: 9; VH: SEQ ID NO: 11)
(d) 1107 (heavy chain CDRs: SEQ ID NOs: 73, 78 and 80; light chain CDRs: SEQ ID NOs: 90, 91, and 95; VL: SEQ ID NO: 13; VH: SEQ ID NO: 15)
(e) ADC-1013 (heavy chain CDRs: SEQ ID NOs: 81, 82 and 83; light chain CDRs: SEQ ID NOs: 96, 97, and 98; VL: SEQ ID NO: 17; VH: SEQ ID NO: 19)
(f) APX005 (heavy chain CDRs: SEQ ID NOs: 84, 85 and 86; light chain CDRs: SEQ ID NOs: 99, 100, and 101; VL: SEQ ID NO: 21; VH: SEQ ID NO: 23)
(g) 21.4.1 (heavy chain CDRs: SEQ ID NOs: 87, 88 and 89; light chain CDRs: SEQ ID NOs: 102, 103, and 104; VL: SEQ ID NO: 25, VH: SEQ ID NO: 27)

The numbering of the antibody (e.g. Antibody X/Y) defines the heavy chain variable region (X) and the light chain variable region (Y), respectively (or, where a single number is indicated, the heavy chain variable region [X] only is defined). As described above, the sequences may be one or more CDR sequence, or the VH and/or VL sequence. As described above, the sequences of the bispecific polypeptide may comprise specified mutations.

In one embodiment binding domain B1 is specific for CD40, typically human CD40 and may comprise any one, two, three, four, five or all six features independently selected from the following:

(a) a heavy chain CDR1 sequence which consists of the sequence “G, F, T, F, S, S, Y, A”;
(b) a heavy chain CDR2 sequence which is 8 amino acids in length and comprises the consensus sequence: “I, G/S, S/G, Y/S, G/S, G/S, G/Y/S, T”;
(c) a heavy chain CDR3 sequence which is 9 to 12 amino acids in length and which comprises the consensus sequence of: “A, R, Y/R/G, Y/P/V/-, N/S/V, F/Y/W, G/H/S, -/S, -/V, M/F, D, Y”
(d) a light chain CDR1 sequence which consists of the sequence: “Q, S, I, S, S, Y”;
(e) a light chain CDR2 sequence which consists of the sequence: “A, A, S”;
(f) a light chain CDR3 sequence which is 9 amino acids in length and comprises the consensus sequence: “Q,Q, Y/S, G/Y, R/S/V, N/A/Y/T, P, P/F/Y, T”.

Binding domain B1 may comprise at least a heavy chain CDR3 as defined in (c) and/or a light chain CDR3 as defined in (f). Binding domain B1 may comprise all three heavy chain CDR sequences of (a), (b) and (c) and/or all three light chain CDR sequences of (d), (e) and (f).

Examples of complete heavy and light chain variable region amino acid sequences for binding domain B1 are shown in Table A. Exemplary nucleic acid sequences encoding each amino acid sequence are also shown. The numbering of said VH and VL regions in Table A corresponds to the numbering system used as in Table C(1) and C(2). Thus, for example, the amino acid sequence for “1132, light chain VL (also known as 1133)” is an example of a complete VL region sequence comprising all three CDRs of VL number 1132 (1133) shown in Table C(2) and the amino acid sequence for “1132, heavy chain VH” is an example of a complete VH region sequence comprising all three CDRs of VH number 1132 shown in Table C(1).

In exemplary embodiments, binding domain B1 comprises:

(a) the three CDRs of the heavy chain and/or the three CDRs of the light chain of antibody 1132/1133 (SEQ ID NOs: 73, 74 and 75; and/or SEQ ID NOs: 90, 91, and 92);
(b) the three CDRs of the heavy chain and/or the three CDRs of the light chain of antibody 1150/1151 (SEQ ID NOs: 73, 76 and 77; and/or SEQ ID NOs:90, 91, and 93);
(c) the three CDRs of the heavy chain and/or the three CDRs of the light chain of antibody 1140/1135 (SEQ ID NOs: 73, 78 and 79; and/or SEQ ID NOs: 90, 91, and 94);
(d) the three CDRs of the heavy chain and/or the three CDRs of the light chain of antibody 1107/1108 (SEQ ID NOs: 73, 78 and 80; and/or SEQ ID NOs: 90, 91, and 95);
(e) the three CDRs of the heavy chain and/or the three CDRs of the light chain of antibody ADC-1013 (SEQ ID NOs: 81, 82 and 83; and/or SEQ ID NOs: 96, 97, and 98);
(f) the three CDRs of the heavy chain and/or the three CDRs of the light chain of antibody APX005 (SEQ ID NOs: 84, 85 and 86; and/or SEQ ID NOs: 99, 100, and 101); or
(g) the three CDRs of the heavy chain and/or the three CDRs of the light chain of antibody 21.4.1 (SEQ ID NOs: 87, 88 and 89; and/or SEQ ID NOs: 102, 103, and 104).

Thus, binding domain B1 may comprise:

(a) the heavy chain variable region and/or the light chain variable region of antibody 1132/1133 (SEQ ID NO: 3 and/or SEQ ID NO: 1);
(b) the heavy chain variable region and/or the light chain variable region of antibody 1150/1151 (SEQ ID NO: 7 and/or SEQ ID NO: 5);
(c) the heavy chain variable region and/or the light chain variable region of antibody 1140/1135 (SEQ ID NO: 11 and/or SEQ ID NO: 9);
(d) the heavy chain variable region and/or the light chain variable region of antibody 1107/1108 (SEQ ID NO:15 and/or SEQ ID NO: 13);
(e) the heavy chain variable region and/or the light chain variable region of antibody ADC-1013 (SEQ ID NO: 19 and/or SEQ ID NO: 17);
(f) the heavy chain variable region and/or the light chain variable region of antibody APX005 (SEQ ID NO: 23 and/or SEQ ID NO: 21); or
(g) the heavy chain variable region and/or the light chain variable region of antibody 21.4.1 (SEQ ID NO: 27 and/or SEQ ID NO: 25).

In exemplary embodiments, binding domain B1 comprises:

the three CDRs of the heavy chain and/or the three CDRs of the light chain of antibody 1132/1133 (SEQ ID NOs: 73, 74 and 75 and/or SEQ ID NOs: 90, 91, and 92), or the exemplary heavy and light chain variable regions (SEQ ID NO: 3 and SEQ ID NO: 1), or heavy and light antibody chains, which comprise said CDRs, as detailed above.

The numbering of the antibody (e.g. Antibody X/Y) defines the heavy chain variable region (X) and the light chain variable region (Y), respectively (or, where a single number is indicated, the heavy chain variable region [X] only is defined).

It will be appreciated by persons skilled in the art that the bispecific polypeptides of the invention may alternatively comprise variants of the above-defined variable regions (or variants of the above CDR sequences).

A variant of any one of the heavy or light chain amino acid sequences or CDR sequences recited herein may be a substitution, deletion or addition variant of said sequence. A variant may comprise 1, 2, 3, 4, 5, up to 10, up to 20, up to 30 or more amino acid substitutions and/or deletions from the said sequence. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | |
|---|---|
| Ala, A | aliphatic, hydrophobic, neutral |
| Cys, C | polar, hydrophobic, neutral |
| Asp, D | polar, hydrophilic, charged (−) |
| Glu, E | polar, hydrophilic, charged (−) |
| Phe, F | aromatic, hydrophobic, neutral |
| Gly, G | aliphatic, neutral |
| His, H | aromatic, polar, hydrophilic, charged (+) |
| Ile, I | aliphatic, hydrophobic, neutral |
| Lys, K | polar, hydrophilic, charged (+) |
| Leu, L | aliphatic, hydrophobic, neutral |
| Met, M | hydrophobic, neutral |
| Asn, N | polar, hydrophilic, neutral |
| Pro, P | hydrophobic, neutral |
| Gln, Q | polar, hydrophilic, neutral |
| Arg, R | polar, hydrophilic, charged (+) |
| Ser, S | polar, hydrophilic, neutral |
| Thr, T | polar, hydrophilic, neutral |
| Val, V | aliphatic, hydrophobic, neutral |
| Trp, W | aromatic, hydrophobic, neutral |
| Tyr, Y | aromatic, polar, hydrophobic |

Amino acids herein may be referred to by full name, three letter code or single letter code.

Preferred “derivatives” or “variants” include those in which instead of the naturally occurring amino acid the amino acid which appears in the sequence is a structural analog thereof. Amino acids used in the sequences may also be derivatised or modified, e.g. labelled, providing the function of the polypeptide is not significantly adversely affected.

Derivatives and variants as described above may be prepared during synthesis of the polypeptide or by post-production modification, or when the polypeptide is in recombinant form using the known techniques of site-directed mutagenesis, random mutagenesis, or enzymatic cleavage and/or ligation of nucleic acids.

Preferably variants have an amino acid sequence which has more than 60%, or more than 70%, e.g. 75 or 80%, preferably more than 85%, e.g. more than 90 or 95% amino acid identity to a sequence as shown in the sequences disclosed herein. This level of amino acid identity may be seen across the full length of the relevant SEQ ID NO sequence or over a part of the sequence, such as across 20, 30, 50, 75, 100, 150, 200 or more amino acids, depending on the size of the full-length polypeptide.

In connection with amino acid sequences, “sequence identity” refers to sequences which have the stated value when assessed using ClustalW (Thompson et al., 1994; the disclosures of which are incorporated herein by reference) with the following parameters:

Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatised.

Thus, in one embodiment binding domain B1 may comprise one or more variants of the above-defined light chain variable regions and/or said heavy chain variable regions having at least 90% sequence identity thereto. Binding domain B1 may also comprise variants of the CDR sequences specified herein, for example variants where up one, two, three, four or five amino acid residues are substituted, deleted to added compared to the specified reference sequences.

In one embodiment, binding domain B1 comprises the light chain of antibody 1132/1133 (SEQ ID NO: 182) and/or the heavy chain of antibody 1132/1133 (SEQ ID NO: 181).

### Tumour Cell-Targeting Domains

The bispecific polypeptides of the invention further comprise a binding domain (B2) which is capable of specifically binding a tumour cell-associated antigen.

By “tumour cell-associated antigen” (also known as a “tumour antigen” or “TAA”) we include proteins accessible on the extracellular surface of tumour cells, such that they are accessible to the bispecific polypeptides of the invention following administration into the body. In one embodiment, the tumour cell-associated antigen is tumour-specific, i.e. it is found exclusively on tumour cells and not on normal, healthy cells. However, it will be appreciated by persons skilled in the art that the tumour cell-associated antigen may be preferentially expressed on tumour cells, i.e. it is expressed on tumour cells at a higher level than on normal, healthy cells (thus, expression of the antigen on tumour cells may be at least five times more than on normal, healthy cells, for example expression levels on tumour cells of at least ten times more, twenty times more, fifty times more or greater).

In one embodiment, binding domain B2 binds to a tumour cell-associated antigen selected from the group consisting of:

(a) products of mutated oncogenes and tumour suppressor genes;
(b) overexpressed or aberrantly expressed cellular proteins;
(c) tumour antigens produced by oncogenic viruses;
(d) oncofetal antigens;
(e) altered cell surface glycolipids and glycoproteins;
(f) cell type-specific differentiation antigens;
(g) hypoxia-induced antigens;
(h) tumour peptides presented by MHC class I;
(i) epithelial tumour antigens;
(j) haematological tumour-associated antigens;
(k) cancer testis antigens; and
(l) melanoma antigens.

Thus, the tumour cell-associated antigen may be selected from the group consisting of 5T4, CD20, CD19, MUC-1, carcinoembryonic antigen (CEA), CA-125, CO17-1A, EpCAM, HER2, HERS, EphA2, EphA3, DR4, DR5, FAP, OGD2, VEGFR, EGFR, NY-ESO-1, survivin, TROP2 and WT-1.

In one embodiment, the tumour cell-associated antigen is selected from the group consisting of 5T4, CD20, EpCAM, EGFR and HER2.

In one embodiment, the tumour cell-associated antigen is EpCAM.

In an alternative embodiment, the tumour cell-associated antigen is not RSV, ROR1, PSMA or mesothelin.

In one embodiment, the tumour cell-associated antigen is an oncofetal antigen. For example, the tumour cell-associated antigen may be 5T4.

In one embodiment, the tumour cell is a solid tumour cell.

For example, the solid tumour may be selected from the groups consisting of renal cell carcinoma, colorectal cancer, lung cancer, prostate cancer, breast cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

Advantageously, binding domain B2 binds to the tumour cell-associated antigen with a K_{D} of less than 100×10⁻⁹M, for example less than 10×10⁻⁹M or less than 5×10⁻⁹M.

Exemplary CDR sequences of binding domain B2 are recited in Tables D(1) and D(2), SEQ ID NOs: 90, 91 and 105 to 160.

In one embodiment, binding domain B2 comprises one or more light chain CDR sequences selected from those in Table D(2), and/or one or more heavy chain CDR sequences selected from Table D(1).

Preferred TAA binding domains may comprise at least a heavy chain CDR3 as defined in any individual row of Table D(1) and/or a light chain CDR3 as defined in in any individual row of Table D(2). Binding domain B2 may comprise all three heavy chain CDR sequences shown in any individual row of Table D(1) (that is, all three heavy chain CDRs of a given “Antibody ref”) and/or all three light chain CDR sequences shown in an individual row of Table D(2) (that is, all three light chain CDRs of a given “Antibody ref”).

Thus, in one embodiment binding domain B2 comprises one, two or three light chain CDR sequences from a particular row for an individual antibody reference in Table D(2), and/or one, two or three heavy chain CDR sequences from the corresponding row for the antibody with the same reference in Table D(1). For example, binding domain B2 might comprise one or more of the light chain CDR sequences for Solitomab (SEQ ID NOs: 136, 137 and 138) and one or more of the heavy chain CDR sequences for Solitomab (SEQ ID NOs: 105, 106 and 107).

Accordingly, in one embodiment binding domain B2 comprises all six CDR sequences for a given antibody (VH/VL) reference, for example binding domain B2 might comprise all six CDR sequences of the ‘Solitomab’ antibody.

Examples of complete heavy and light chain variable region amino acid sequences are shown in Table B. Exemplary nucleic acid sequences encoding each amino acid sequence are also shown. The numbering of said VH and VL regions in Table B corresponds to the numbering system used as in Table D(1) and D(2). Thus, for example, the amino acid sequence for “Solitomab, light chain VL” is an example of a complete VL region sequence comprising all three CDRs of Antibody ref Solitomab shown in Table D(2) and the amino acid sequence for “Solitomab, heavy chain VH” is an example of a complete VH region sequence comprising all three CDRs of Antibody ref Solitomab shown in Table D(1).

In one embodiment, binding domain B2 comprises a VH and/or a VL amino acid sequence as given in Table B. In one embodiment, binding domain B2 comprises a VH and VL amino acid sequence as given in Table B for a particular antibody reference. For example, binding domain B2 may comprise the VH sequence of Solitomab (SEQ ID NO: 31) and/or the VL sequence of Solitomab (SEQ ID NO: 29).

In one embodiment binding domain B2 binds EpCAM. In one embodiment, binding domain B2 is specific for EpCAM and comprises one or more CDR sequences selected from the groups consisting of:

(a) EpCAM heavy chain CDRs, SEQ ID NOs: 105 to 120; and/or
(b) EpCAM light chain CDRs, SEQ ID NOs: 90, 91 and 136 to 147.

In one embodiment the EpCAM binding domain of B2 is selected from: Solitomab, 005025, 005038, Adecatumumab, 4D5MOCB, and 3-171.

Thus, the CDR or VH and VL sequences of binding domain B2 might be selected from antibodies from the group consisting of:

(a) Solitomab (heavy chain CDRs: SEQ ID NOs: 105, 106 and 107; light chain CDRs: SEQ ID NOs: 136, 137, and 138; VL: SEQ ID NO:29; VH: SEQ ID NO: 31)
(b) 005025 (heavy chain CDRs: SEQ ID NOs: 108, 109 and 110; light chain CDRs: SEQ ID NOs: 90, 91, and 139; VL: SEQ ID NO: 35; VH: SEQ ID NO: 36)
(c) 005038 (heavy chain CDRs: SEQ ID NOs: 108, 109 and 111; light chain CDRs: SEQ ID NOs: 90, 91, and 140; VL: SEQ ID NO: 39; VH: SEQ ID NO: 40)
(d) Adecatumumab (heavy chain CDRs: SEQ ID NOs: 112, 113 and 114; light chain CDRs: SEQ ID NOs: 90, 137, and 141; VL: SEQ ID NO: 41; VH: SEQ ID NO: 43)
(e) 4D5MOCB (heavy chain CDRs: SEQ ID NOs: 115, 116 and 117; light chain CDRs: SEQ ID NOs: 142, 143, and 144; VL: SEQ ID NO: 45; VH: SEQ ID NO: 47)
(f) 3-171 (heavy chain CDRs: SEQ ID NOs: 118, 119 and 120; light chain CDRs: SEQ ID NOs: 145, 146, and 147; VL: SEQ ID NO: 49; VH: SEQ ID NO: 51)

As described above, the sequences may be one or more CDR sequence, or the VH and/or VL sequence. In exemplary embodiments, binding domain B2 comprises the three CDRs of the light chain and/or the three CDRs of the heavy chain of an antibody selected from: Solitomab, 005025, 005038, Adecatumumab, 4D5MOCB, and 3-171.

In an alternative embodiment, binding domain B2 does not bind EpCAM.

In one embodiment binding domain B2 binds HER2. In one embodiment, binding domain B2 is specific for HER2 and comprises one or more CDR sequences selected from the groups consisting of:

(a) HER2 heavy chain CDRs, SEQ ID NOs: 121 to 126; and/or
(b) HER2 light chain CDRs, SEQ ID NOs: 148 to 152.

In one embodiment the HER2 binding domain of B2 is selected from: Trastuzumab and Pertuzumab.

Thus, the CDR or VH and VL sequences of binding domain B2 might be selected from antibodies from the group consisting of:

(a) Trastuzumab (heavy chain CDRs: SEQ ID NOs: 121, 122 and 123; light chain CDRs: SEQ ID NOs: 148, 149, and 150; VL: SEQ ID NO: 53; VH: SEQ ID NO: 55)
(b) Pertuzumab (heavy chain CDRs: SEQ ID NOs: 124, 125 and 126; light chain CDRs: SEQ ID NOs: 151, 149, and 152; VL: SEQ ID NO: 57; VH: SEQ ID NO: 59)

As described above, the sequences may be one or more CDR sequence, or the VH and/or VL sequence.

In one embodiment binding domain B2 binds 5T4, for example binding domain B2 may be the 5T4 binding domain of 2992. Thus, binding domain B2 may comprise any of the sequences of 2992, as follows: heavy chain CDRs: SEQ ID NOs: 127, 128 and 129; light chain CDRs: SEQ ID NOs: 153, 91, and 154; VL: SEQ ID NO: 61; VH: SEQ ID NO: 63).

In one embodiment binding domain B2 binds CD20, for example binding domain B2 may be the CD20 binding domain of Rituximab. Thus, binding domain B2 may comprise any of the sequences of Rituximab, as follows: heavy chain CDRs: SEQ ID NOs: 130, 131 and 132; light chain CDRs: SEQ ID NOs: 155, 156, and 157; VL: SEQ ID NO: 65; VH: SEQ ID NO: 67).

In one embodiment binding domain B2 binds EGFR, for example binding domain B2 may be the EGFR binding domain of Cetuximab. Thus, binding domain B2 may comprise any of the sequences of Cetuximab, as follows: heavy chain CDRs: SEQ ID NOs: 133, 134 and 135; light chain CDRs: SEQ ID NOs: 158, 159, and 160; VL: SEQ ID NO: 69; VH: SEQ ID NO: 71).

As described above, the sequences may be one or more CDR sequence, or the VH and/or VL sequence.

In alternative embodiments, B2 can comprise CDRs selected from known antibodies to tumour associated antigens. For example, B2 may comprise the CDRs of an antibody to EpCAM, such as Edrecolomab (as disclosed in U.S. Pat. No. 7,557,190, the disclosure of which is incorporated herein by reference). Alternatively, B2 may comprise the CDRs of an antibody to EGFR, such as Panitumumab (as disclosed in U.S. Pat. No. 6,235,883, the disclosure of which is incorporated herein by reference). In a further embodiment, B2 may comprise the CDRs of an antibody to CD20, such as Ofatumumab (Drug Bank, Accession number: DB 06650, the disclosure of which is incorporated herein by reference). In a further embodiment, B2 may comprise the CDRs of a commercially available antibody to HER2.

Alternatively, B2 can comprise the heavy chain variable regions and/or light chain variable regions selected from known antibodies to tumour associated antigens, for example antibodies to CD20, EpCAM, EGFR and HER2, as described above.

It will be appreciated by skilled persons that binding domain B2 may alternatively comprise variants of said light chain variable regions and/or said heavy chain variable regions, for example having at least 90% sequence identity thereto. Binding domain B2 may also comprise variants of the CDR sequences specified herein, for example variants where up to one, two, three, four or five amino acid residues are substituted, deleted or added compared to the specified reference sequences. Variants are as described above in relation to binding domain B1.

Alternatively, B2 can comprise the heavy chain and/or light chain selected from known antibodies to tumour associated antigens, for example antibodies to CD20, EpCAM, EGFR and HER2, as described above.

For reference, the antibody reference used in this application, possible alternative names for the same antibody/binding domain, and the target of the antibody/binding domain, is laid out in Table 1 below.

TABLE 1
Alternative names for particular antibodies/binding domains,
and the relevant target for each antibody/binding domain.

| Antibody reference | Alternative names | Target |
|---|---|---|
| 1132 | 1132/1133 | CD40 |
| 1150 | 1150/1151 | CD40 |
| 1140 | 1140/1135 | CD40 |
| 1107 | 1107/1108 | CD40 |
| ADC-1013 | G12 | CD40 |
| APX005 | | CD40 |
| 21.4.1 | | CD40 |
| Solitomab | 3174 | EpCAM |
| | BM2 | |
| 005025 | | EpCAM |
| 005038 | | EpCAM |
| Adecatumumab | | EpCAM |
| 4D5MOCB | 3188 | EpCAM |
| | BM1 | |
| 3-17I | | EpCAM |
| Trastuzumab | Herceptin | HER2 |
| Pertuzumab | | HER2 |
| 2992 | 1210 | 5T4 |
| | 1210LO1 | |
| | 2992/2993 | |
| Rituximab | | CD20 |
| Cetuximab | | EGFR |

### Exemplary CD40-Tumour Cell-Associated Antigen Bispecific Antibodies

In one embodiment of the bispecific polypeptides of the invention, binding domain B1 is an IgG and binding domain B2 is an scFv. Conversely, binding domain B1 may be an scFv and binding domain B2 may be an IgG.

In one embodiment binding domain B1 is an immunoglobulin and binding domain B2 is a Fab. Conversely, binding domain B1 may be a Fab and binding domain B2 may be an immunoglobulin. The bispecific polypeptide may optionally be in the RUBY™ format. The bispecific polypeptide format is as described above and as laid out in FIG. 2, and the bispecific polypeptide may comprise certain mutations as described above.

Bispecific polypeptides of the invention may comprise the CDRs of the light chains of any of the B1 domains described above (as laid out in Table C(2) below), and/or the CDRs of the heavy chains of any of the B1 domains described above (as laid out in Table C(1) below), in combination with any of the CDRs of the light chains of any of the B2 domains described above (as laid out in Table D(2)), and/or the CDRs of the heavy chains of any of the B2 domains described above (as laid out in Table D(1)).

Thus, in certain embodiments B1 and B2 comprise the respective variable regions comprising the CDRs identified above. For example, B1 may comprise the heavy chain variable region and/or the light chain variable region of antibody 1132/1133 (SEQ ID NO: 3 and/or SEQ ID NO: 1) and B2 may comprise the heavy chain variable region and/or the light chain variable region of antibody Solitomab (SEQ ID NO: 31 and/or SEQ ID NO: 29).

For example, in one embodiment of the invention B2 comprises the 3 CDRs of the light chain of antibody Solitomab and/or the 3 CDRs of the heavy chain of antibody Solitomab (SEQ ID NOs: 136, 137, and 138 and/or SEQ ID NOs 105, 106 and 107) or the corresponding heavy chain variable region and/or light chain variable region (SEQ ID NO: 31 and SEQ ID NO: 29 and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of antibody 005025 and/or the 3 CDRs of the heavy chain of antibody 005025 (SEQ ID NOs: 90, 91, and 139; and/or SEQ ID NOs 108, 109 and 110) or the corresponding heavy chain variable region and/or light chain variable region (SEQ ID NO: 36 and SEQ ID NO: 35); and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of antibody 005038 and/or the 3 CDRs of the heavy chain of antibody 005038 (SEQ ID NOs: 90, 91, and 140 and/or SEQ ID NOs 108, 109 and 111) or the corresponding heavy chain variable region and/or light chain variable region (SEQ ID NO: 40 and SEQ ID NO: 39); and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of antibody 2992/2993 and/or the 3 CDRs of the heavy chain of antibody 2992/2993 (SEQ ID NOs: 153, 91, and 154 and/or SEQ ID NOs 127, 128 and 129) or the corresponding heavy chain variable region and/or light chain variable region (SEQ ID NO: 63 and SEQ ID NO: 61) and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of a commercially available antibody to CD20, as described above, and/or the 3 CDRs of the heavy chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of a commercially available antibody to EpCAM, as described above, and/or the 3 CDRs of the heavy chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of a commercially available antibody to EGFR, as described above, and/or the 3 CDRs of the heavy chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In a further embodiment of the invention B2 comprises the 3 CDRs of the light chain of a commercially available antibody to HER2, as described above, and/or the 3 CDRs of the heavy light chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table A.

In one embodiment the bispecific polypeptide of the invention binds CD40 and EpCAM. For example, in one embodiment of the bispecific polypeptide of the invention: B1 comprises the three CDRs of the heavy chain and/or the three CDRs of the light chain of antibody 1132 (SEQ ID NOs: 73, 74 and 75 and/or SEQ ID NOs: 90, 91, and 92) and B2 comprises the three CDRs of the heavy chain and/or the three CDRs of the light chain of antibody Solitomab (SEQ ID NOs: 105, 106 and 107 and/or SEQ ID NOs: 136, 137, and 138).

Such a CD40-EpCAM bispecific polypeptide may optionally be in the RUBY™ format. Thus, the CD40 binding domain B1 is an immunoglobulin, and the EpCAM binding domain B2 is a Fab fragment. The bispecific polypeptide format is as described above and as laid out in FIG. 2, and the bispecific polypeptide may comprise certain mutations as described above.

In one embodiment the bispecific polypeptide does not bind CD40 and EpCAM.

In one embodiment the bispecific polypeptide of the invention binds CD40 and 5T4. Thus, in one embodiment of the bispecific polypeptide of the invention B1 comprises the three CDRs of the heavy chain and/or the three CDRs of the light chain of antibody 1132 (SEQ ID NOs: 73, 74 and 75 and/or SEQ ID NOs: 90, 91, and 92) and B2 comprises the three CDRs of the heavy chain and/or the three CDRs of the light chain of antibody 2992 (SEQ ID NOs: 127, 128 and 129 and/or SEQ ID NOs: 153, 91, and 154).

Such a CD40-5T4 bispecific polypeptide may optionally be in the RUBY™ format. Thus, the CD40 binding domain B1 is an immunoglobulin, and the 5T4 binding domain B2 is a Fab fragment. The bispecific polypeptide format is as described above and as laid out in FIG. 2, and the bispecific polypeptide may comprise certain mutations as described above.

In one embodiment the bispecific polypeptide of the invention binds CD40 and HER2. Thus, in one embodiment of the bispecific polypeptide of the invention B1 comprises the three CDRs of the heavy chain and/or the three CDRs of the light chain of antibody 1132 (SEQ ID NOs: 73, 74 and 75 and/or SEQ ID NOs: 90, 91, and 92) and B2 comprises the three CDRs of the heavy chain and/or the three CDRs of the light chain of antibody Trastuzumab (SEQ ID NOs: 121, 122 and 123 and/or SEQ ID NOs: 148, 149, and 150).

Such a CD40-HER2 bispecific polypeptide may optionally be in the RUBY™ format. Thus, the CD40 binding domain B1 is an immunoglobulin, and the HER2 binding domain B2 is a Fab fragment. The bispecific polypeptide format is as described above and as laid out in FIG. 2, and the bispecific polypeptide may comprise certain mutations as described above.

The B1 domain may comprise the light chain variable region and/or the heavy chain variable region of any B1 domain described above, and the B2 domain may comprise the light chain variable region and/or the heavy chain variable region of any B2 domain described above, or variants of said light chain variable regions and/or said heavy chain variable regions having at least 90% sequence identity thereto.

Typically, the bispecific polypeptides of the invention will comprise constant region sequences, in addition to the above-defined variable region sequences. Bispecific polypeptides of the invention may be in any suitable format. For example, bispecific polypeptides may be in the RUBY™ format (as described above, and shown in FIG. 2), or in the Morrison format.

An exemplary heavy chain constant region amino acid sequence which may be combined with any VH region sequence disclosed herein (to form a complete heavy chain) is the following IgG1 heavy chain constant region sequence:

[SEQ ID NO: 174]
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK

Likewise, an exemplary light chain constant region amino acid sequence which may be combined with any VL region sequence disclosed herein (to form a complete light chain) is the Kappa chain constant region sequence reproduced here:

[SEQ ID NO: 175]
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC

Other light chain constant region sequences are known in the art and could also be combined with any VL region disclosed herein.

In one embodiment, the polypeptide may comprise the following constant region amino acid sequences:

(a) Reference sequence CH1 (SEQ ID NO: 179):
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSC
(wherein the bold and underlined section is part
of the hinge region, but is present in the Fab
fragment)
and/or
(b) Reference sequence CKappa (SEQ ID NO: 180):
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC

As described above, these reference sequences may comprise one or more mutations to prevent the formation of aggregates and/or a Fab by-product. Such mutation positions (identified earlier in the description) may be given relative to any of the above constant region sequences.

In one embodiment, the bispecific polypeptide is in the RUBY™ format, comprising an immunoglobulin and a Fab fragment, wherein the Fab fragment is fused to the C-terminus of the heavy chain of the immunoglobulin via the light chain of the Fab fragment.

Thus in one embodiment, binding domain B1 is an immunoglobulin, and binding domain B2 is a Fab fragment, and the Fab fragment is fused to the C-terminus of the heavy chain of the immunoglobulin via the light chain of the Fab fragment. Additionally, the bispecific polypeptide comprises one or more mutations selected from the group consisting of:

| | |
|---|---|
| VH | X44R/E/D/K, X49C, X120K |
| VL | X44R/E/D/K, X49D, X120C |
| CH1 | H168A/G, F170G/A, L145Q, S183V, T187E/D, |
| CKappa/CLambda | S/T114A, V133T, L135Y/W, N/S137K/R/H, |
| | S176W/V/Y |

*numbering according to IMGT system for VH/VL domains and according to EU numbering system for constant domains
*X refers to any amino acid

| | |
|---|---|
| VH | X44R/E/D/K, X49C, X120K |
| VL | X44R/E/D/K, X49D, X120C |
| CH1 | H168A/G, F170G/A, L145Q, S183V, T187E/D, |
| CKappa/CLambda | S/T114A, V133T, L135Y/W, N/S137K/R/H, |
| | S176W/V/Y |

*numbering according to IMGT system for VH/VL domains and according to Kabat numbering system for constant domains
*X refers to any amino acid

In one embodiment of the invention, B1 binds CD40 and B1 comprises a heavy chain comprising the sequence of SEQ ID NO: 181, and/or a light chain comprising the sequence of SEQ ID NO: 182. These sequences are the full chain sequences for 1132.

In one embodiment B2 binds EpCAM and comprises a heavy chain comprising the sequence of SEQ ID NO: 183, and/or a light chain comprising the sequence of SEQ ID NO: 184. These sequences are the full chain sequences for Solitomab.

Accordingly, in one embodiment, the bispecific polypeptide is an CD40-EpCAM bispecific polypeptide, wherein B1 comprises the heavy chain sequence of 1132 in the RUBY™ format (SEQ ID NO:181) and the light chain sequence of 1132 in the RUBY™ format (SEQ ID NO: 182) and B2 comprises the heavy chain sequence of Solitomab in the RUBY™ format (SEQ ID NO: 183) and the light chain sequence of Solitomab in the RUBY™ format (SEQ ID NO: 184). Thus, combined SEQ ID NOs: 181 to 184 represent a 1132-Solitomab LALA-mutated bsAb in RUBY™ format, wherein B1 is an 1132 IgG and B2 is a Solitomab Fab fragment.

Exemplary full heavy and light chain sequences
Binding domain B1:
Heavy chain (SEQ ID NO: 181):
EVQLLESGGGLVQPGGSLRLSCAASGFTFSSYAMSWVRRAPGKGLE
WVSGIGSYGGGTYYADSVKGRFTISRDNSKNTLYLQMNSLRAEDTA
VYYCARYVNFGMDYWGQGTLVTVSSASTKGPSVFPLAPSSKSTSGG
TAALGCLVKDYFPEPVTVSWNSGALTSGVATGPAVLQSSGLYSLSS
VVTVPSSSLGTQTYICNVNHKPSNTKVDKKVEPKSCDKTHTCPPCP
APEAAGGPSVFLFPPKPKDTLMISRTPEVTCVVVDVSHEDPEVKFN
WYVDGVEVHNAKTKPREEQYNSTYRVVSVLTVLHQDWLNGKEYKCK
VSNKALPAPIEKTISKAKGQPREPQVYTLPPSRDELTKNQVSLTCL
VKGFYPSDIAVEWESNGQPENNYKTTPPVLDSDGSFFLYSKLTVDK
SRWQQGNVFSCSVMHEALHNHYTQKSLSLSPGK
Light chain (SEQ ID NO: 182):
DIQMTQSPSSLSASVGDRVTITCRASQSISSYLNWYQEKPGKAPKL
LIYAASSLQSGVPSRFSGSGSGTDFTLTISSLQPEDFATYYCQQYG
RNPPTFGQGTKLEIKRTVAAPSVFIFPPSDEQLKSGTASVVCYLNN
FYPREAKVQWKVDNALQSGNSQESVTEQDSKDSTYSLWSTLTLSKA
DYEKHKVYACEVTHQGLSSPVTKSFNRGEC
Binding domain 82:
Heavy chain (SEQ ID NO: 183):
EVQLLEQSGAELVRPGTSVKISCKASGYAFTNYWLGWVKERPGHGL
EWIGDIFPGSGNIHYNEKFKGKATLTADKSSSTAYMQLSSLTFEDS
AVYFCARLRNWDEPMDYWGQGTTVTVSSASTKGPSVFPLAPSSKST
SGGTAALGCLVKDYFPEPVTVSWNSGALTSGVHTFPAVLQSSGLYS
LSSVVEVPSSSLGTQTYICNVNHKPSNTKVDKKVEPKSC
Light chain (SEQ ID NO: 184):
ELVMTQSPSSLTVTAGEKVTMSCKSSQSLLNSGNQKNYLTWYQRKP
GQPPKLLIYWASTRESGVPDRFTGSGSGTDFTLTISSVQAEDLAVY
YCQNDYSYPLTFGAGTKLEIKRTVAAPAVFIFPPSDEQLKSGTASV
VCLLKNFYPREAKVQWKVDNALQSGNSQESVTEQDSKDSTYSLSST
LTLSKADYEKHKVYACEVTHQGLSSPVTKSFNRGEC

As discussed above, methods for the production of polypeptides of the invention are well known in the art.

Conveniently, the polypeptide is or comprises a recombinant polypeptide. Suitable methods for the production of such recombinant polypeptides are well known in the art, such as expression in prokaryotic or eukaryotic hosts cells (for example, see Green & Sambrook, 2012, Molecular Cloning, A Laboratory Manual, Fourth Edition, Cold Spring Harbor, N.Y., the relevant disclosures in which document are hereby incorporated by reference).

Polypeptides of the invention can also be produced using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

It will be appreciated by persons skilled in the art that polypeptides of the invention may alternatively be synthesised artificially, for example using well known liquid-phase or solid phase synthesis techniques (such as t-Boc or Fmoc solid-phase peptide synthesis).

### Polynucleotides, Vectors and Cells

A second aspect of the invention provides an isolated nucleic acid molecule encoding a bispecific polypeptide according to any one of the preceding claims, or a component polypeptide chain thereof. For example, the nucleic acid molecule may comprise any of the nucleotide sequences provided in Tables A and B.

Thus, a polynucleotide of the invention may encode any polypeptide as described herein, or all or part of B1 or all or part of B2. The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Representative polynucleotides which encode examples of a heavy chain or light chain amino acid sequence of an antibody may comprise or consist of any one of the nucleotide sequences disclosed herein, for example the sequences set out in Tables A and B.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al, 1984; the disclosures of which are incorporated herein by reference).

The PILEUP and BLAST algorithms can also be used to calculate homology or line up sequences (typically on their default settings), for example as described in Altschul, 1993; Altschul et al, 1990, the disclosures of which are incorporated herein by reference).

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992; the disclosures of which are incorporated herein by reference) alignments (B) of 50, expectation (E) of 10, M=5, N=4, and a comparison of both strands.

The BLAST algorithm performs a statistical analysis of the similarity between two sequences; see e.g. Karlin & Altschul, 1993; the disclosures of which are incorporated herein by reference. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

A polypeptide of the invention may thus be produced from or delivered in the form of a polynucleotide which encodes, and is capable of expressing, it.

Polynucleotides of the invention can be synthesised according to methods well known in the art, as described by way of example in Green & Sambrook (2012, Molecular Cloning—a laboratory manual, 4^{th} edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art (see Green & Sambrook, supra).

The invention also includes cells that have been modified to express a polypeptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a polypeptide of the invention include mammalian HEK293T, CHO, HeLa, NS0 and COS cells. Preferably the cell line selected will be one which is not only stable, but also allows for mature glycosylation and cell surface expression of a polypeptide.

Such cell lines of the invention may be cultured using routine methods to produce a polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

In one embodiment, the nucleic acid molecule encodes an antibody heavy chain or variable region thereof.

In one embodiment, the nucleic acid molecule encodes an antibody light chain or variable region thereof.

By “nucleic acid molecule” we include DNA (e.g. genomic DNA or complementary DNA) and mRNA molecules, which may be single- or double-stranded. By “isolated” we mean that the nucleic acid molecule is not located or otherwise provided within a cell.

In one embodiment, the nucleic acid molecule is a cDNA molecule.

It will be appreciated by persons skilled in the art that the nucleic acid molecule may be codon-optimised for expression of the antibody polypeptide in a particular host cell, e.g. for expression in human cells (for example, see Angov, 2011, the disclosures of which are incorporated herein by reference).

Also included within the scope of the invention are the following:

(a) a third aspect of the invention provides a vector (such as an expression vector) comprising a nucleic acid molecule according to the second aspect of the invention;
(b) a fourth aspect of the invention provides a host cell (such as a mammalian cell, e.g. human cell, or Chinese hamster ovary cell, e.g. CHOK1SV cells) comprising a nucleic acid molecule according to the second aspect of the invention or a vector according to the third aspect of the invention; and
(c) a fifth aspect of the invention provides a method of making a polypeptide according to the first aspect of the invention comprising culturing a population of host cells according to the fourth aspect of the invention under conditions in which said polypeptide is expressed, and isolating the polypeptide therefrom.

### Methods of Production

In a sixth aspect, the present invention provides compositions comprising molecules of the invention, such as the antibodies, bispecific polypeptides, polynucleotides, vectors and cells described herein. For example, the invention provides a composition comprising one or more molecules of the invention, such as one or more antibodies and/or bispecific polypeptides of the invention, and at least one pharmaceutically acceptable carrier.

It will be appreciated by persons skilled in the art that additional compounds may also be included in the pharmaceutical compositions, including, chelating agents such as EDTA, citrate, EGTA or glutathione.

The pharmaceutical compositions may be prepared in a manner known in the art that is sufficiently storage stable and suitable for administration to humans and animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the CD40 and TAA-binding activity of the bispecific polypeptide of the invention. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the polypeptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the polypeptide of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The polypeptides of the invention may be formulated into any type of pharmaceutical composition known in the art to be suitable for the delivery thereof.

In one embodiment, the pharmaceutical compositions of the invention may be in the form of a liposome, in which the polypeptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polyvinyl imidazole, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the polypeptide may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active polypeptide. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonar, buccal, oral, parenteral, vaginal and rectal. Also administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques.

They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration.

Alternatively, the pharmaceutical compositions may be administered intranasally or by inhalation (for example, in the form of an aerosol spray presentation from a pressurised container, pump, spray or nebuliser with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas). In the case of a pressurised aerosol, the dosage unit may be determined by providing a valve to deliver a metered amount. The pressurised container, pump, spray or nebuliser may contain a solution or suspension of the active polypeptide, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

The pharmaceutical compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the dose may be provided as a continuous infusion over a prolonged period.

Particularly preferred compositions are formulated for systemic administration.

The composition may preferably be formulated for sustained release over a period of time. Thus the composition may be provided in or as part of a matrix facilitating sustained release. Preferred sustained release matrices may comprise a montanide or γ-polyglutamic acid (PGA) nanoparticles.

The bispecific polypeptides can be formulated at various concentrations, depending on the efficacy/toxicity of the polypeptide being used. For example, the formulation may comprise the active polypeptide at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the active polypeptide at a concentration of between 300 μM and 700 μM.

Typically, the therapeutic dose of the bispecific polypeptide (with or without a therapeutic moiety) in a human patient will be in the range of 100 μg to 700 mg per administration (based on a body weight of 70 kg). For example, the maximum therapeutic dose may be in the range of 0.1 to 10 mg/kg per administration, e.g. between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly or monthly.

It will be appreciated by persons skilled in the art that the pharmaceutical compositions of the invention may be administered alone or in combination with other therapeutic agents used in the treatment of cancers, such as antimetabolites, alkylating agents, anthracyclines and other cytotoxic antibiotics, vinca alkyloids, etoposide, platinum compounds, taxanes, topoisomerase I inhibitors, other cytostatic drugs, antiproliferative immunosuppressants, corticosteroids, sex hormones and hormone antagonists, and other therapeutic antibodies (such as antibodies against a tumour cell-associated antigen or an immune checkpoint modulator).

For example, the pharmaceutical compositions of the invention may be administered in combination with an immunotherapeutic agent that binds a target selected from the group consisting of PD-1/PD-L1, CTLA-4, CD137, OX40, GITR, LAG3, TIM3, CD27, VISTA and KIR.

Thus, the invention encompasses combination therapies comprising a bispecific polypeptide of the invention together with a further immunotherapeutic agent, effective in the treatment of cancer, which specifically binds to an immune checkpoint molecule. It will be appreciated that the therapeutic benefit of the further immunotherapeutic agent may be mediated by attenuating the function of an inhibitory immune checkpoint molecule and/or by activating the function of a stimulatory immune checkpoint or co-stimulatory molecule.

In one embodiment, the further immunotherapeutic agent is selected from the group consisting of:

(a) an immunotherapeutic agent that inhibits the function of PD-1 and/or PD-L1;
(b) an immunotherapeutic agent that inhibits the function of CTLA-4;
(c) an immunotherapeutic agent that activates the function of CD137;
(d) an immunotherapeutic agent that binds activates the function of OX40;
(e) an immunotherapeutic agent that inhibits the function of LAG3;
(f) an immunotherapeutic agent that inhibits the function of TIM3; and
(g) an immunotherapeutic agent that inhibits the function of VISTA.

Thus, the further immunotherapeutic agent may be a PD-1 inhibitor, such as an anti-PD-1 antibody, or antigen-binding fragment thereof capable of inhibiting PD-1 function (for example, Nivolumab, Pembrolizumab, Lambrolizumab, PDR-001, MEDI-0680 and AMP-224). Alternatively, the PD-1 inhibitor may comprise or consist of an anti-PD-L1 antibody, or antigen-binding fragment thereof capable of inhibiting PD-1 function (for example, Durvalumab, Atezolizumab, Avelumab and MDX-1105).

In another embodiment, the further immunotherapeutic agent is a CTLA-4 inhibitor, such as an anti-CTLA-4 antibody or antigen-binding portion thereof.

In a further embodiment, the further immunotherapeutic agent activates CD137, such as an agonistic anti-CD137 antibody or antigen-binding portion thereof.

In a further embodiment, the further immunotherapeutic agent activates OX40, such as an agonistic anti-OX40 antibody or antigen-binding portion thereof.

In a further embodiment, the further immunotherapeutic agent inhibits the function of LAG3, TIM3 or VISTA (Lines et al. 2014).

It will be appreciated by persons skilled in the art that the presence of the two active agents (as detailed above) may provide a synergistic benefit in the treatment of a tumour in a subject. By “synergistic” we include that the therapeutic effect of the two agents in combination (e.g. as determined by reference to the rate of growth or the size of the tumour) is greater than the additive therapeutic effect of the two agents administered on their own. Such synergism can be identified by testing the active agents, alone and in combination, in a relevant cell line model of the solid tumour.

Also within the scope of the present invention are kits comprising polypeptides or other compositions of the invention and instructions for use. The kit may further contain one or more additional reagents, such as an additional therapeutic or prophylactic agent as discussed above.

### Medical Uses and Methods

The polypeptides in accordance with the present invention may be used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

Thus, a seventh aspect of the invention provides a bispecific polypeptide according to the first aspect of the invention for use in medicine.

An eighth aspect of the invention provides a bispecific polypeptide according to the first aspect of the invention for use in treating a neoplastic disorder in a subject.

By ‘treatment’ we include both therapeutic and prophylactic treatment of the patient. The term ‘prophylactic’ is used to encompass the use of an agent, or formulation thereof, as described herein which either prevents or reduces the likelihood of a neoplastic disorder, or the spread, dissemination, or metastasis of cancer cells in a patient or subject. The term ‘prophylactic’ also encompasses the use of an agent, or formulation thereof, as described herein to prevent recurrence of a neoplastic disorder in a patient who has previously been treated for the neoplastic disorder.

In one embodiment, the neoplastic disorder is associated with the formation of solid tumours within the subject's body.

Thus, the solid tumour may be selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

For example, the solid tumour may be selected from the groups consisting of renal cell carcinoma, colorectal cancer, lung cancer, prostate cancer and breast cancer.

In one embodiment, the neoplastic disorder is selected from the groups consisting of epithelial cancer, bladder cancer, breast cancer, cervical cancer, colorectal cancer, gastric cancer, esophageal cancer, head cancer, neck cancer, head and neck cancer, non-small cell lung cancer, mesothelioma, lung cancer, cervical cancer, endometrial cancer, ovarian cancer, stomach cancer, pancreatic cancer, prostate cancer, gastrointestinal caner and renal cancer.

In one embodiment, the neoplastic disorder is selected from the groups consisting of bladder cancer, breast cancer, cervical cancer, colorectal cancer, gastric cancer, head cancer, neck cancer, head and neck cancer, non-small cell lung cancer, mesothelioma, ovarian cancer, pancreatic cancer, prostate cancer, and renal cancer; and the TAA is 5T4.

In one embodiment, the neoplastic disorder is selected from the groups consisting of epithelial cancer; esophageal cancer, gastric cancer, colorectal cancer, stomach cancer, pancreatic cancer, breast cancer, lung cancer, ovarian cancer, gastrointestinal caner and bladder cancer; and

the TAA is EpCAM.

In one embodiment, the neoplastic disorder is selected from the groups consisting of breast cancer, esophageal cancer, lung cancer, cervical cancer, endometrial cancer, ovarian cancer, bladder cancer, pancreatic cancer, stomach cancer;

the TAA is Her2.

In one embodiment, the solid tumour is selected from the groups consisting of epithelial, bladder, breast, cervical, colorectal, gastric, esophageal, head, neck, head and neck, non-small cell lung, mesothelioma, lung, cervical, endometrial, ovarian, stomach, pancreatic, prostate, gastrointestinal and renal.

In one embodiment, the solid tumour is selected from the groups consisting of bladder, breast, cervical, colorectal, gastric, head, neck, head and neck, non-small cell lung, ovarian, pancreatic, prostate, and renal; and

the TAA is 5T4.

In one embodiment, the solid tumour is selected from the groups consisting of epithelial; esophageal, gastric, colorectal, stomach, pancreatic, breast, lung, ovarian, gastrointestinal and bladder; and

the TAA is EpCAM.

In one embodiment, the solid tumour is selected from the groups consisting of breast, esophageal, lung, cervical, endometrial, ovarian, bladder, pancreatic, stomach;

the TAA is Her2.

A ninth aspect of the invention provides a use of a bispecific polypeptide according to the first aspect of the invention in the preparation of a medicament for treating or preventing a neoplastic disorder in a subject.

In one embodiment, the neoplastic disorder is associated with the formation of solid tumours within the subject's body (for example, as detailed above).

A tenth aspect of the invention provides a method for the treatment or diagnosis of a neoplastic disorder in a subject, comprising the step of administering to the subject an effective amount of a bispecific polypeptide according to the first aspect of the invention.

In one embodiment, the neoplastic disorder is associated with the formation of solid tumours within the subject's body (for example, as detailed above).

In one embodiment, the subject is human.

In one embodiment, the method comprises administering the bispecific polypeptide systemically.

In one embodiment, the methods further comprise administering to the subject one or more additional therapeutic agents.

The listing or discussion of an apparently prior-published document in this specification should not necessarily be taken as an acknowledgement that the document is part of the state of the art or is common general knowledge.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the above description and the accompanying drawings. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The following drawings form part of the present specification and are included to further demonstrate certain aspects of the present invention. The invention may be better understood by reference to one or more of these drawings in combination with the detailed description of specific embodiments presented herein.

### BRIEF DESCRIPTION OF FIGURES

Preferred, non-limiting examples which embody certain aspects of the invention will now be described, with reference to the following figures:

FIG. 1 shows a schematic representation of the structure of exemplary formats for a bispecific antibody of the invention. In each format, the constant regions are shown as filled light grey; variable heavy chain regions VH1 are shown as chequered black and white; variable light chain regions VL1 are shown as filled white; variable heavy chain regions VH2 are shown as filled black; and variable light chain regions VL2 are shown as white with diagonal lines. CD40 binding domains (binding domain 1) are typically represented as a pair of a chequered black and white domain with a filled white domain (VH1/VL1); tumour cell-associated antigen binding domains (binding domain 2) are typically represented as a pair of a filled black domain and a white domain with diagonal lines (VH2/VL2). However, in all of the formats shown, it will be appreciated that binding domains 1 and 2 may be switched. That is, a CD40 binding domain may occur in a position shown in this figure for a tumour cell-associated antigen-binding domain, and vice versa.

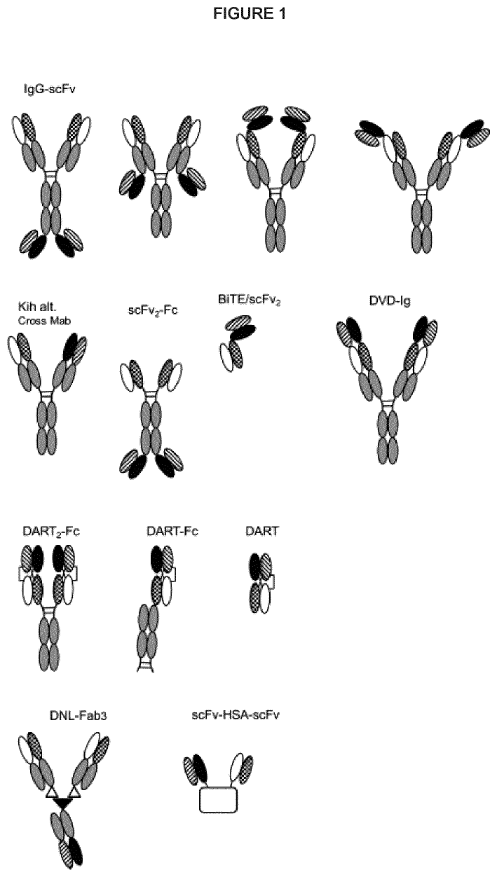

FIG. 2 shows an example composition of a bispecific antibody construct. The bispecific antibody of FIG. 2 is made up of three types of polypeptide chains: (1) IgG heavy chains (white) fused to Fab light chains (chequered) via a polypeptide linker. (2) IgG light chains (bricked) and (3) Fab heavy chains (black). Mutations are introduced in the interface between heavy and light chains.

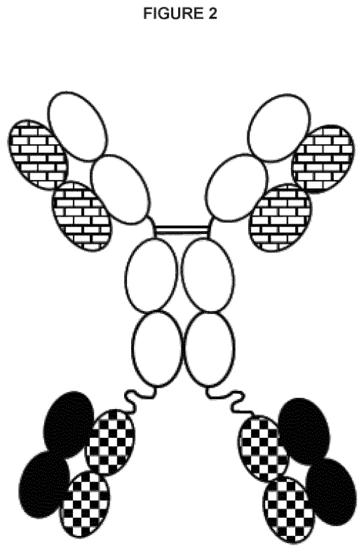

FIG. 3. ELISA analyses showing the binding of bsAbs to human EpCAM. Mono ELISA (top frame) shows that 1132-005025.M, 1132-005038.M and 1132-3188.M bind stronger to EpCAM compared to 1132-3174.M. Dual ELISA (bottom frame) shows that higher maximum signal is obtained with 1132-3174.R (RUBY™ format) compared to 1132-3174.M (Morrison format).

FIG. 4. Binding of CD40-EpCAM bispecific antibodies to EpCAM expressed on cells. 1132-005025.M, 1132-005038.M, 1132-3174.M and 1132-3188.M were incubated with EpCAM-expressing cell lines. Binding of antibodies to cells was analysed by flow cytometry using anti-human IgG detection antibody.

FIG. 5. Binding of the CD40-EpCAM bispecific antibody 1132-3174.R and CD40 monospecific antibody 1132.m2 to EpCAM-transfected and control-transfected CHO cells. Binding of biotinylated antibodies was detected by flow cytometry using fluorochrome-conjugated streptavidin. Results are pooled from two replicates in one representative experiment of two.

FIG. 6. Binding of the CD40-EpCAM bispecific antibody 1132-3174.R and CD40 monospecific antibody 1132.m2 to EpCAM-expressing tumour cell lines, HT29, JEG, JAR and BxPC3. Binding of biotinylated antibodies was detected by flow cytometry using fluorochrome-conjugated streptavidin. Results are pooled from two replicates in one representative experiment of two.

FIG. 7. Binding of the CD40-EpCAM bispecific antibody 1132-3174.R and CD40 monospecific antibody 1132.m2 to cell populations among PBMC, monocytes, B cells, T cells and NK cells. PBMC were incubated with biotinylated 1132-3174.R and 1132.m2 along with fluorochrome-conjugated antibodies directed against CD19, CD14, CD3 and CD56. Binding of biotinylated antibodies to different cell populations was detected by flow cytometry using fluorochrome-conjugated streptavidin. Results are pooled from three donors in one representative experiment of two.

FIG. 8. Effect of the CD40-EpCAM bispecific antibodies 1132-3174.M, 1132-005038.M, 1132-005025.M and 1132-3188.M on B cell activation. Primary human B cells were cultured with titrated antibodies in the presence or absence of EpCAM expressed on CHO cells. After 2 days, expression of CD86 on B cells was analysed by FACS. The graphs show pooled results from 3 donors (1132-3174.M, 1132-005038.M and 1132-005025.M) or 2 donors (1132-3188.M).

FIG. 9. Effect of the CD40-EpCAM bispecific antibody 1132-3174.R on B cell activation. Primary human B cells were cultured with titrated antibodies in the presence or absence of EpCAM expressed on CHO cells. After 2 days, expression of CD86 on B cells was analysed by FACS. The graph shows pooled results from three donors in one representative experiment of two.

FIG. 10. Effect of the CD40-5T4 bispecific antibody 1132-1210.M on B cell proliferation. Primary human B cells were cultured with titrated antibodies in the presence or absence of 5T4. After 2 days, B cell proliferation was analysed using Promega's CellTiter-Glo Luminescent cell viability assay.

FIG. 11. Effect of the CD40-EpCAM bispecific antibodies 1132-3174.M and 1132-3174.R on DC activation. Human monocyte-derived DCs were cultured with titrated antibodies in the presence or absence of EpCAM expressed on CHO cells. After 2 days, expression of CD86 and HLA-DR on CD14− CD1a+ DCs was analysed by FACS. The graph shows pooled results from six donors in four experiments.

FIG. 12. Effect of the CD40-EpCAM bispecific antibodies 1132-3174.M and 1132-3174.R on IL-12p40 production by DCs. Human monocyte-derived DCs were cultured with titrated antibodies in the presence or absence of EpCAM expressed on CHO cells. After 2 days, supernatants were collected and IL-12p40 content was analysed by ELISA. The graph shows pooled results from six donors in four experiments.

FIG. 13. Effect of the CD40-EpCAM bispecific antibodies 1132-3174.M and 1132-3174. R on internalization of EpCAM+ tumour cell debris in a CD40+ cell line. Fluorescently labelled EpCAM+ tumour cell debris was incubated with fluorescently labelled CD40+ Raji cells and titrated antibodies. Images were captured using a live cell imaging system and the number of tumour cell debris localized in CD40+ cells was analysed. The graph displays the mean of two replicates after three hours of incubation in one representative experiment of three.

FIG. 14. Effect of the anti-CD40 monoclonal antibodies 1132/1133, 1140/1135 and 1150/1151 on the activation of antigen-presenting cells determined by the expression of CD80 and CD86. hCD40tg mice were dosed with 100 μg of the indicated treatments at the start of the experiment and three days later. Spleens were collected one day after the final dose and analysed by flow cytometry for the expression of CD80 and CD86 on dendritic cells (CD11c+ MHCII+) and B cells (CD19+ MHCII+).

FIG. 15. Antibody localization to tumour tissue determined by frequency of human IgG (hlgG)-positive cells. hCD40tg or non-hCD40tg C57Bl/6 mice inoculated with MB49-hEpCAM tumours were dosed with the indicated treatments on day 10 post-inoculation. Tumours were collected one day later, stained with anti-hlgG antibody and analysed by flow cytometry.

FIG. 16. Antibody localization to tumour tissue determined by frequency of human IgG (hlgG)-positive cells. hCD40tg mice inoculated with B16 tumours, which were either h5T4 positive or negative, were dosed with the indicated treatments on days 16 and 19 post-inoculation. Tumours were collected on day 20, stained with anti-hlgG antibody and analysed by flow cytometry.

FIG. 17. MB49 tumour growth. hCD40tg mice inoculated with MB49 tumours, which were either hEpCAM positive or negative, were dosed with the indicated treatments on days 7, 10 and 13 post-inoculation. Tumours were frequently measured until the first mouse in any of the treatment groups reached a tumour volume above the ethical limit.

FIG. 18. MB49 tumour growth. hCD40tg mice inoculated with MB49 tumours, which were either hEpCAM positive or negative, were dosed with the indicated treatments on days 10, 13 and 16 post-inoculation. Tumours were frequently measured until the first mouse in any of the treatment groups reached a tumour volume above the ethical limit.

FIG. 19. Individual MB49-wt and MB49-hEpCAM tumour growth. Naïve hCD40tg mice, or mice previously cured from MB49-hEpCAM tumours (rechallenged), were inoculated with two MB49 tumours, one hEpCAM positive and one hEpCAM negative (wt) on each side of the flank. Tumours were frequently measured, and the tumour volume plotted over time.

FIG. 20. Effect of 1132, 1132.m2, 1132-3174.R and an anti-CD40 reference antibody on spleen weight. hCD40tg mice were inoculated with MB49-hEpCAM tumours and administered with the indicated treatment doses on days 10, 13 and 16 post-inoculation. Spleens were collected four days after the final dose and weighed.

FIG. 21. Effect of 1132, 1132.m2, 1132-3174.R and an anti-CD40 reference antibody on plasma levels of IL-6. hCD40tg mice were inoculated with MB49-hEpCAM tumours and administered with the indicated treatment doses on days 10, 13 and 16 post-inoculation. Blood was collected 4 hrs after the treatments on days 10 and 13, and plasma was obtained from the blood. IL-6 levels were measured in the plasma samples by ELISA.

FIG. 22. Individual MB49-wt and Panc02 tumour growth. Naïve hCD40tg mice, or mice previously cured from MB49-hEpCAM tumours (rechallenged), were inoculated with an MB49-wt tumour and a Panc02 tumour, on each side of the flank. Tumours were frequently measured, and the tumour volume plotted over time.

FIG. 23. Effect of 1132-3174.R on the proliferation of OVA-specific T cells in vitro. CTV-labeled OT-1 T cells were cultured with hCD40tg DC and necrotic MB49-hEpCAM-OVA or MB49-wt cells in the presence of 1132-3174.R or culture medium control. The frequency of proliferating cells (CTV low) among CD8+ T cells was analyzed after three days of culture.

FIG. 24. Effect of 1132-3174.R on the frequency of proliferating OVA-specific T cells. hCD40tg mice, which had received CTV-labeled CD8+ T cells isolated from OT-1 mice, were immunized with heat-shocked MB49-hEpCAM-OVA cells and administered 167 μg 1132-3174.R. Four days later, spleens and inguinal lymph nodes were analysed by flow cytometry for assessment of the frequency of proliferating OVA-specific T cells.

FIG. 25. Effect of 1132-3174.R on the frequency of OVA-specific T cells. MB49-hEpCAM-OVA tumour-bearing hCD40tg mice, which had received CTV-labeled CD8+ T cells isolated from OT-1 mice, were administered 417 μg 1132-3174.R and also 20 ug FTY720 to prevent egress of OT-1 T cells primed in tumour-draining lymph nodes. On day 21 post-inoculation, tumour-draining (inguinal) lymph nodes were analysed by flow cytometry for assessment of the frequency of OVA-specific T cells.

FIG. 26. Quantification of human 5T4 on the transfected murine CT26 cell line. Three clones were identified with low, intermediate and high density of human 5T4.

FIG. 27. Effect of the CD40-5T4 bispecific antibody 1132-1210.M on internalization of 5T4+ tumour cell debris in a CD40+ cell line. Fluorescently-labeled CT26-wt or CT26-h5T4 (low, intermediate or high) tumour cell debris were incubated with fluorescently-labeled CD40+ Raji cells and titrated bispecific and/or monoclonal antibody. Images were captured using a live cell imaging system and the number of tumour cell debris localized in CD40+ cells was analyzed. The graphs display the mean of two replicates after 12 hours of incubation in one representative experiment of three. The bispecific antibody 1132-1210.M demonstrates an increased localization of CT26-5T4^{hi} tumour cell debris to CD40+ cells compared to the monoclonal antibody 1132.m2 (A). A 5T4 density of at least between 0.15×10⁶-1×10⁶ molecules per cell is required for effective localization of tumour debris to CD40+ cells as demonstrated for 1132-1210.M (B).

FIG. 28. Quantification of human EpCAM on the tumour cell lines BxPC3, MCF7, JAR and JEG.

FIG. 29. Effect of the CD40-EpCAM bispecific antibody 1132-3174.R on internalization of EpCAM+ tumour cell debris in a CD40+ cell line. Fluorescently-labeled BxPC3, MCF7, JAR or JEG tumour cell debris were incubated with fluorescently-labeled CD40+ Raji cells and titrated bispecific and/or monoclonal antibody. Images were captured using a live cell imaging system and the number of tumour cell debris localized in CD40+ cells was analyzed. The graphs display the mean of two replicates after 12 hours of incubation in one representative experiment of three. The bispecific antibody 1132-3174.R demonstrates an increased localization of EpCAM^{int} and EpCAM^{hi} tumour cell debris to CD40+ cells compared to the monoclonal antibody 1132.m2 (A). An EpCAM density of at least between 2.5×10⁵-1.5×10⁶ molecules per cell is required for effective localization of tumour debris to CD40+ cells as demonstrated for 1132-3174.R (B).

FIG. 30. Quantification of HER2 on the tumour cell lines BxPC3, HT29, MCF7, LS174T and SK-OV-3.

FIG. 31. Effect of the CD40-HER2 bispecific antibody 1132-Trastuzumab® on internalization of HER2+ tumour cell debris in a CD40+ cell line. Fluorescently-labeled BxPC3, HT29, MCF7, LS174T, LS174T-HER2 KO and SK-OV-3 tumour cell debris were incubated with fluorescently-labeled CD40+ Raji cells and titrated bispecific and/or monoclonal antibody. Images were captured using a live cell imaging system and the number of tumour cell debris localized in CD40+ cells was analyzed. The graphs display the mean of two replicates after 12 hours of incubation in one representative experiment of three. The bispecific antibody 1132-Trastuzumab® demonstrates an increased localization of HER2^{hi} tumour cell debris to CD40+ cells compared to the monoclonal antibody 1132.m2.

FIG. 32. A HER2 density of at least between 1×10⁵-3×10⁶ molecules per tumour cell is required for effective localization as demonstrated for 1132-Trastuzumab®

FIG. 33. Effect of the DEC-205-EpCAM bispecific antibody 3G9-3174.R on internalization of EpCAM+ tumour cell debris in a DEC-205+ cell line. Fluorescently-labeled BxPC3 (EpCAM^{low}), MCF7 (EpCAM^{int}) or JAR (EpCAM^{hi}) tumour cell debris were incubated with fluorescently-labeled DEC-205+ Raji cells and 1.2 nM of 3G9-3174.R or 1188-3174.R, an isotype-EpCAM bispecific antibody. Images were captured using a live cell imaging system and the number of tumour cell debris localized in DEC-205+ cells was analyzed. The graphs display the mean of two replicates after 0-12 hours of incubation in one experiment of two. The bispecific antibody 3G9.3174.R demonstrates an increased localization of EpCAM^{int} tumour cell debris from MCF7 (A) and EpCAM^{hi} tumour cell debris from JAR cells (B) to DEC-205+ cells compared to 1188-3174.R. This effect is not observed with EpCAM^{low} tumour cell debris from BxPC3 cells (C).

FIG. 34. Dynamic Light Scattering (DLS) profile of isolated MB49-EpCAM-OVA-derived exosomes. Exosomes isolated from the culture supernatant of MB49-EpCAM-OVA were analysed by DLS using Uncle.

FIG. 35. Effect of 1132-3174.R on the proliferation of OVA-specific T cells in vitro. CTV-labeled OT-1 T cells were cultured with hCD40tg DC and MB49-hEpCAM-OVA-derived exosomes in the presence of 1132-3174.R or 1188-3174. The frequency of proliferating cells (CTV low) among CD8+ T cells was analyzed after three days of culture.

FIG. 36. Survival of MB49 tumour-bearing mice. hCD40tg mice inoculated with MB49 tumours, which were either hEpCAM positive or negative, were dosed with the indicated treatments on days 10, 13 and 16 post-inoculation. Mice were kept in the study until their tumour volume reached the ethical limit of 2000 mm³, at which point the mice were sacrificed.

### TABLES (SEQUENCES)

TABLE A
Binding domain B1 VL and VH amino acid (aa) and nucleotide (nt) sequences

| SEQ | | | |
|---|---|---|---|
| ID | | | |
| NO. | ANTIBODY REF | TYPE | SEQUENCE |
| 1 | 1132, light chain | aa | DIQMTQSPSSLSASVGDRVTITCRASQSISSYLN |
| | VL (also known | | WYQQKPGKAPKLLIYAASSLQSGVPSRFSGSG |
| | as 1133) | | SGTDFTLTISSLQPEDFATYYCQQYGRNPPTFG |
| | | | QGTKLEIK |
| 2 | 1132, light chain | nt | gatattcagatgacccagagcccgagcagcctgagcgcgagcgt |
| | VL (also known | | gggcgatcgcgtgaccattacctgccgcgcgagccagagcatta |
| | as 1133) | | gcagctatctgaactggtatcagcagaaaccgggcaaagcgcc |
| | | | gaaactgctgatttatgcggcgagcagcctgcagagcggcgtgc |
| | | | cgagccgctttagcggcagcggcagcggcaccgattttaccctga |
| | | | ccattagcagcctgcagccggaagattttgcgacctattattgccag |
| | | | cagtatggccgcaacccgccgacctttggccagggcaccaaact |
| | | | ggaaattaaa |
| 3 | 1132, heavy | aa | EVQLLESGGGLVQPGGSLRLSCAASGFTFSSY |
| | chain VH | | AMSWVRQAPGKGLEWVSGIGSYGGGTYYADS |
| | | | VKGRFTISRDNSKNTLYLQMNSLRAEDTAVYYC |
| | | | ARYVNFGMDYWGQGTLVTVSS |
| 4 | 1132, heavy | nt | gaagtgcagctgctggaaagcggcggcggcctggtgcagccgg |
| | chain VH | | gcggcagcctgcgcctgagctgcgcggcgagcggctttaccttta |
| | | | gcagctatgcgatgagctgggtgcgccaggcgccgggcaaagg |
| | | | cctggaatgggtgagcggcattggcagctatggcggcggcacct |
| | | | attatgcggatagcgtgaaaggccgctttaccattagccgcgataa |
| | | | cagcaaaaacaccctgtatctgcagatgaacagcctgcgcgcgg |
| | | | aagataccgcggtgtattattgcgcgcgctatgtgaactttggcatg |
| | | | gattattggggccagggcaccctggtgaccgtgagcagc |
| 5 | 1150, light chain | aa | DIQMTQSPSSLSASVGDHVTITCRASQSISSYLN |
| | VL (also known | | WYQQKPGKAPKLLIYAASSLQSGVPSRFSGSG |
| | as 1151) | | SGTDFTLTISSLQPEDFATYYCQQYGSAPPTFG |
| | | | QGTKLEIK |
| 6 | 1150, light chain | nt | gatattcagatgacccagagcccgagcagcctgagcgcgagcgt |
| | VL (also known | | gggcgatcatgtgaccattacctgccgcgcgagccagagcattag |
| | as 1151) | | cagctatctgaactggtatcagcagaaaccgggcaaagcgccg |
| | | | aaactgctgatttatgcggcgagcagcctgcagagcggcgtgcc |
| | | | gagccgctttagcggcagcggcagcggcaccgattttaccctgac |
| | | | cattagcagcctgcagccggaagattttgcgacctattattgccagc |
| | | | agtatggcagcgcgccgccgacctttggccagggcaccaaactg |
| | | | gaaattaaa |
| 7 | 1150, heavy | aa | EVQLLESGGGLVQPGGSLRLSCAASGFTFSSY |
| | chain VH | | AMSWVRQAPGKGLEWVSGIGGSSSYTSYADS |
| | | | VKGRFTISRDNSKNTLYLQMNSLRAEDTAVYYC |
| | | | ARYYSYHMDYWGQGTLVTVSS |
| 8 | 1150, heavy | nt | gaagtgcagctgctggaaagcggcggcggcctggtgcagccgg |
| | chain VH | | gcggcagcctgcgcctgagctgcgcggcgagcggctttaccttta |
| | | | gcagctatgcgatgagctgggtgcgccaggcgccgggcaaagg |
| | | | cctggaatgggtgagcggcattggcggcagcagcagctatacca |
| | | | gctatgcggatagcgtgaaaggccgctttaccattagccgcgata |
| | | | acagcaaaaacaccctgtatctgcagatgaacagcctgcgcgcg |
| | | | gaagataccgcggtgtattattgcgcgcgctattatagctatcatatg |
| | | | gattattggggccagggcaccctggtgaccgtgagcagc |
| 9 | 1140, light chain | aa | DIQMTQSPSSLSASVGDRVTITCRASQSISSYLN |
| | VL (also known | | WYQQKPGKAPKLLIYAASSLQSGVPSRFSGSG |
| | as 1135) | | SGTDFTLTISSLQPEDFATYYCQQSYSTPYTFG |
| | | | QGTKLEIK |
| 10 | 1140, light chain | nt | gatattcagatgacccagagcccgagcagcctgagcgcgagcgt |
| | VL (also known | | gggcgatcgcgtgaccattacctgccgcgcgagccagagcatta |
| | as 1135) | | gcagctatctgaactggtatcagcagaaaccgggcaaagcgcc |
| | | | gaaactgctgatttatgcggcgagcagcctgcagagcggcgtgc |
| | | | cgagccgctttagcggcagcggcagcggcaccgattttaccctga |
| | | | ccattagcagcctgcagccggaagattttgcgacctattattgccag |
| | | | cagagctatagcaccccgtatacctttggccagggcaccaaactg |
| | | | gaaattaaa |
| 11 | 1140, heavy | aa | EVQLLESGGGLVQPGGSLRLSCAASGFTFSSY |
| | chain VH | | AMSWVRQAPGKGLEWVSAISGSGGSTYYADS |
| | | | VKGRFTISRDNSKNTLYLQMNSLRAEDTAVYYC |
| | | | ARGPVYSSVFDYWGQGTLVTVSS |
| 12 | 1140, heavy | nt | gaagtgcagctgctggaaagcggcggcggcctggtgcagccgg |
| | chain VH | | gcggcagcctgcgcctgagctgcgcggcgagcggctttaccttta |
| | | | gcagctatgcgatgagctgggtgcgccaggcgccgggcaaagg |
| | | | cctggaatgggtgagcgcgattagcggcagcggcggcagcacc |
| | | | tattatgcggatagcgtgaaaggccgctttaccattagccgcgata |
| | | | acagcaaaaacaccctgtatctgcagatgaacagcctgcgcgcg |
| | | | gaagataccgcggtgtattattgcgcgcgcggcccggtgtatagc |
| | | | agcgtgtttgattattggggccagggcaccctggtgaccgtgagca |
| | | | gc |
| 13 | 1107, light chain | aa | DIQMTQSPSSLSASVGDRVTITCRASQSISSYLN |
| | VL (also known | | WYQQKPGKAPKLLIYAASSLQSGVPSRFSGSG |
| | as 1108) | | SGTDFTLTISSLQPEDFATYYCQQYGVYPFTFG |
| | | | QGTKLEIK |
| 14 | 1107, light chain | nt | gatattcagatgacccagagcccgagcagcctgagcgcgagcgt |
| | VL (also known | | gggcgatcgcgtgaccattacctgccgcgcgagccagagcatta |
| | as 1108) | | gcagctatctgaactggtatcagcagaaaccgggcaaagcgcc |
| | | | gaaactgctgatttatgcggcgagcagcctgcagagcggcgtgc |
| | | | cgagccgctttagcggcagcggcagcggcaccgattttaccctga |
| | | | ccattagcagcctgcagccggaagattttgcgacctattattgccag |
| | | | cagtatggcgtgtatccgtttacctttggccagggcaccaaactgg |
| | | | aaattaaa |
| 15 | 1107, heavy | aa | EVQLLESGGGLVQPGGSLRLSCAASGFTFSSY |
| | chain VH | | AMSWVRQAPGKGLEWVSAISGSGGSTYYADS |
| | | | VKGRFTISRDNSKNTLYLQMNSLRAEDTAVYYC |
| | | | ARRVWGFDYWGQGTLVTVSS |
| 16 | 1107, heavy | nt | gaagtgcagctgctggaaagcggcggcggcctggtgcagccgg |
| | chain VH | | gcggcagcctgcgcctgagctgcgcggcgagcggctttaccttta |
| | | | gcagctatgcgatgagctgggtgcgccaggcgccgggcaaagg |
| | | | cctggaatgggtgagcgcgattagcggcagcggcggcagcacc |
| | | | tattatgcggatagcgtgaaaggccgctttaccattagccgcgata |
| | | | acagcaaaaacaccctgtatctgcagatgaacagcctgcgcgcg |
| | | | gaagataccgcggtgtattattgcgcgcgccgcgtgtggggctttg |
| | | | attattggggccagggcaccctggtgaccgtgagcagc |
| 17 | ADC-1013, light | aa | QSVLTQPPSASGTPGQRVTISCTGSSSNIGAGY |
| | chain VL | | NVYWYQQLPGTAPKLLIYGNINRPSGVPDRFSG |
| | | | SKSGTSASLAISGLRSEDEADYYCAAWDKSISG |
| | | | LVFGGGTKLTVLG |
| 18 | ADC-1013, light | nt | cagagcgtgctgacccagccgccgagcgcgagcggcaccccg |
| | chain VL | | ggccagcgcgtgaccattagctgcaccggcagcagcagcaaca |
| | | | ttggcgcgggctataacgtgtattggtatcagcagctgccgggcac |
| | | | cgcgccgaaactgctgatttatggcaacattaaccgcccgagcgg |
| | | | cgtgccggatcgctttagcggcagcaaaagcggcaccagcgcg |
| | | | agcctggcgattagcggcctgcgcagcgaagatgaagcggatta |
| | | | ttattgcgcggcgtgggataaaagcattagcggcctggtgtttggcg |
| | | | gcggcaccaaactgaccgtgctgggg |
| 19 | ADC-1013, | aa | EVQLLESGGGLVQPGGSLRLSCAASGFTFSTY |
| | heavy chain VH | | GMHWVRQAPGKGLEWLSYISGGSSYIFYADSV |
| | | | RGRFTISRDNSENALYLQMNSLRAEDTAVYYCA |
| | | | RILRGGSGMDLWGQGTLVTVSS |
| 20 | ADC-1013, | nt | gaagtgcagctgctggaaagcggcggcggcctggtgcagccgg |
| | heavy chain VH | | gcggcagcctgcgcctgagctgcgcggcgagcggctttaccttta |
| | | | gcacctatggcatgcattgggtgcgccaggcgccgggcaaaggc |
| | | | ctggaatggctgagctatattagcggcggcagcagctatattttttat |
| | | | gcggatagcgtgcgcggccgctttaccattagccgcgataacagc |
| | | | gaaaacgcgctgtatctgcagatgaacagcctgcgcgcggaag |
| | | | ataccgcggtgtattattgcgcgcgcattctgcgcggcggcagcgg |
| | | | catggatctgtggggccagggcaccctggtgaccgtgagcagc |
| 21 | APX005, light | aa | DIQMTQSPSSLSASVGDRVTIKCQASQSISSRL |
| | chain VL | | AWYQQKPGKPPKLLIYRASTLASGVPSRFSGS |
| | | | GSGTDFTLTISSLQPEDVATYYCQCTGYGISWP |
| | | | IGGGTKVEIK |
| 22 | APX005, light | nt | gatattcagatgacccagagcccgagcagcctgagcgcgagcgt |
| | chain VL | | gggcgatcgcgtgaccattaaatgccaggcgagccagagcatta |
| | | | gcagccgcctggcgtggtatcagcagaaaccgggcaaaccgcc |
| | | | gaaactgctgatttatcgcgcgagcaccctggcgagcggcgtgcc |
| | | | gagccgctttagcggcagcggcagcggcaccgattttaccctgac |
| | | | cattagcagcctgcagccggaagatgtggcgacctattattgcca |
| | | | gtgcaccggctatggcattagctggccgattggcggcggcaccaa |
| | | | agtggaaattaaa |
| 23 | APX005, heavy | aa | QVQLVESGGGVVQPGRSLRLSCAASGFSFSST |
| | chain VH | | YVCWVRQAPGKGLEWIACIYTGDGTNYSASWA |
| | | | KGRFTISKDSSKNTVYLQMNSLRAEDTAVYFCA |
| | | | RPDITYGFAINFWGPGTLVTVSS |
| 24 | APX005, heavy | nt | caggtgcagctggtggaaagcggcggcggcgtggtgcagccgg |
| | chain VH | | gccgcagcctgcgcctgagctgcgcggcgagcggctttagcttta |
| | | | gcagcacctatgtgtgctgggtgcgccaggcgccgggcaaaggc |
| | | | ctggaatggattgcgtgcatttataccggcgatggcaccaactata |
| | | | gcgcgagctgggcgaaaggccgctttaccattagcaaagatagc |
| | | | agcaaaaacaccgtgtatctgcagatgaacagcctgcgcgcgg |
| | | | aagataccgcggtgtatttttgcgcgcgcccggatattacctatggc |
| | | | tttgcgattaacttttggggcccgggcaccctggtgaccgtgagca |
| | | | gc |
| 25 | 21.4.1, light | aa | DIQMTQSPSSVSASVGDRVTITCRASQGIYSWL |
| | chain VL | | AWYQQKPGKAPNLLIYTASTLQSGVPSRFSGS |
| | | | GSGTDFTLTISSLQPEDFATYYCQQANIFPLTFG |
| | | | GGTKVEIK |
| 26 | 21.4.1, light | nt | gatattcagatgacccagagcccgagcagcgtgagcgcgagcg |
| | chain VL | | tgggcgatcgcgtgaccattacctgccgcgcgagccagggcattt |
| | | | atagctggctggcgtggtatcagcagaaaccgggcaaagcgcc |
| | | | gaacctgctgatttataccgcgagcaccctgcagagcggcgtgcc |
| | | | gagccgctttagcggcagcggcagcggcaccgattttaccctgac |
| | | | cattagcagcctgcagccggaagattttgcgacctattattgccagc |
| | | | aggcgaacatttttccgctgacctttggcggcggcaccaaagtgg |
| | | | aaattaaa |
| 27 | 21.4.1, heavy | aa | QVQLVQSGAEVKKPGASVKVSCKASGYTFTGY |
| | chain VH | | YMHWVRQAPGQGLEWMGWINPDSGGTNYAQ |
| | | | KFQGRVTMTRDTSISTAYMELNRLRSDDTAVYY |
| | | | CARDQPLGYCTNGVCSYFDYWGQGTLVTVSS |
| 28 | 21.4.1, heavy | nt | caggtgcagctggtgcagagcggcgcggaagtgaaaaaaccg |
| | chain VH | | ggcgcgagcgtgaaagtgagctgcaaagcgagcggctatacctt |
| | | | taccggctattatatgcattgggtgcgccaggcgccgggccaggg |
| | | | cctggaatggatgggctggattaacccggatagcggcggcacca |
| | | | actatgcgcagaaatttcagggccgcgtgaccatgacccgcgata |
| | | | ccagcattagcaccgcgtatatggaactgaaccgcctgcgcagc |
| | | | gatgataccgcggtgtattattgcgcgcgcgatcagccgctgggct |
| | | | attgcaccaacggcgtgtgcagctattttgattattggggccagggc |
| | | | accctggtgaccgtgagcagc |

TABLE B
Binding domain B2 VL and VH amino acid (aa) and nucleotide (nt) sequences

| SEQ | | | |
|---|---|---|---|
| ID | | | |
| NO. | ANTIBODY REF | TYPE | SEQUENCE |
| 29 | Solitomab | aa | ELVMTQSPSSLTVTAGEKVTMSCKSSQSLLNS |
| | EpCAM binding | | GNQKNYLTWYQQKPGQPPKLLIYWASTRESGV |
| | domain (BD) | | PDRFTGSGSGTDFTLTISSVQAEDLAVYYCQND |
| | (alternative | | YSYPLTFGAGTKLEIK |
| | name 3174), | | |
| | light chain VL | | |
| 30 | Solitomab | nt | gaactggtgatgacccagagcccgagcagcctgaccgtgaccg |
| | EpCAM BD, light | | cgggcgaaaaagtgaccatgagctgcaaaagcagccagagcc |
| | chain VL | | tgctgaacagcggcaaccagaaaaactatctgacctggtatcag |
| | | | cagaaaccgggccagccgccgaaactgctgatttattgggcgag |
| | | | cacccgcgaaagcggcgtgccggatcgctttaccggcagcggc |
| | | | agcggcaccgattttaccctgaccattagcagcgtgcaggcggaa |
| | | | gatctggcggtgtattattgccagaacgattatagctatccgctgac |
| | | | ctttggcgcgggcaccaaactggaaattaaa |
| 31 | Solitomab | aa | EVQLLEQSGAELVRPGTSVKISCKASGYAFTNY |
| | EpCAM BD, | | WLGWVKQRPGHGLEWIGDIFPGSGNIHYNEKF |
| | heavy chain VH | | KGKATLTADKSSSTAYMQLSSLTFEDSAVYFCA |
| | | | RLRNWDEPMDYWGQGTTVTVSS |
| 32 | Solitomab | nt | gaagtgcagctgctggaacagagcggcgcggaactggtgcgcc |
| | EpCAM BD, | | cgggcaccagcgtgaaaattagctgcaaagcgagcggctatgc |
| | heavy chain VH | | gtttaccaactattggctgggctgggtgaaacagcgcccgggcca |
| | | | tggcctggaatggattggcgatatttttccgggcagcggcaacattc |
| | | | attataacgaaaaatttaaaggcaaagcgaccctgaccgcggat |
| | | | aaaagcagcagcaccgcgtatatgcagctgagcagcctgaccttt |
| | | | gaagatagcgcggtgtatttttgcgcgcgcctgcgcaactgggatg |
| | | | aaccgatggattattggggccagggcaccaccgtgaccgtgagc |
| | | | agc |
| 33 | 005025 full DNA | nt | GAGGTGCAGCTGTTGGAGAGCGGGGGAGGC |
| | seqence | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCAGCTATTA |
| | | | GTGGTAGTGGTGGTAGCACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCGGTTACGCTTCTTTCG |
| | | | TTGGTGGTTACTTTGACTATTGGGGCCAGGG |
| | | | AACCCTGGTCACCGTCTCCTCAGGTGGAGGC |
| | | | GGTTCAGGCGGAGGTGGATCCGGCGGTGGC |
| | | | GGATCGGACATCCAGATGACCCAGTCTCCAT |
| | | | CCTCCCTGAGCGCATCTGTAGGAGACCGCGT |
| | | | CACCATCACTTGCCGGGCAAGTCAGAGCATT |
| | | | AGCAGCTATTTAAATTGGTATCAGCAGAAACC |
| | | | AGGGAAAGCCCCTAAGCTCCTGATCTATGCT |
| | | | GCATCCAGTTTGCAAAGTGGGGTCCCATCAC |
| | | | GTTTCAGTGGCAGTGGAAGCGGGACAGATTT |
| | | | CACTCTCACCATCAGCAGTCTGCAACCTGAA |
| | | | GATTTTGCAACTTATTACTGTCAACAGCCGGG |
| | | | TTCTTCTTCTCCGTACACTTTTGGCCAGGGGA |
| | | | CCAAGCTGGAGATCAAA |
| 34 | 005025 full | aa | EVQLLESGGGLVQPGGSLRLSCAASGFTFSSY |
| | amino acid | | AMSWVRQAPGKGLEWVSAISGSGGSTYYADS |
| | sequence | | VKGRFTISRDNSKNTLYLQMNSLRAEDTAVYYC |
| | | | ARGYASFVGGYFDYWGQGTLVTVSSGGGGSG |
| | | | GGGSGGGGSDIQMTQSPSSLSASVGDRVTITC |
| | | | RASQSISSYLNWYQQKPGKAPKLLIYAASSLQS |
| | | | GVPSRFSGSGSGTDFTLTISSLQPEDFATYYCQ |
| | | | QPGSSSPYTFGQGTKLEIK |
| 35 | 005025 light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQPGSSSPYTF |
| | | | GQGTKLEIK |
| 36 | 005025 heavy | aa | EVQLLESGGGLVQPGGSLRLSCAASGFTFSSY |
| | chain VH | | AMSWVRQAPGKGLEWVSAISGSGGSTYYADS |
| | | | VKGRFTISRDNSKNTLYLQMNSLRAEDTAVYYC |
| | | | ARGYASFVGGYFDYWGQGTLVTVSS |
| 37 | 005038 full DNA | nt | GAGGTGCAGCTGTTGGAGAGCGGGGGAGGC |
| | sequence | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCAGCTATTA |
| | | | GTGGTAGTGGTGGTAGCACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTCTGGTGGTTACTCTG |
| | | | GTGACCATTTTGACTATTGGGGCCAGGGAAC |
| | | | CCTGGTCACCGTCTCCTCAGGTGGAGGCGGT |
| | | | TCAGGCGGAGGTGGATCCGGCGGTGGCGGA |
| | | | TCGGACATCCAGATGACCCAGTCTCCATCCT |
| | | | CCCTGAGCGCATCTGTAGGAGACCGCGTCAC |
| | | | CATCACTTGCCGGGCAAGTCAGAGCATTAGC |
| | | | AGCTATTTAAATTGGTATCAGCAGAAACCAGG |
| | | | GAAAGCCCCTAAGCTCCTGATCTATGCTGCA |
| | | | TCCAGTTTGCAAAGTGGGGTCCCATCACGTT |
| | | | TCAGTGGCAGTGGAAGCGGGACAGATTTCAC |
| | | | TCTCACCATCAGCAGTCTGCAACCTGAAGATT |
| | | | TTGCAACTTATTACTGTCAACAGTCTTACAAC |
| | | | CTGTTCACTTTTGGCCAGGGGACCAAGCTGG |
| | | | AGATCAAA |
| 38 | 005038 full | aa | EVQLLESGGGLVQPGGSLRLSCAASGFTFSSY |
| | amino acid | | AMSWVRQAPGKGLEWVSAISGSGGSTYYADS |
| | sequence | | VKGRFTISRDNSKNTLYLQMNSLRAEDTAVYYC |
| | | | ARSGGYSGDHFDYWGQGTLVTVSSGGGGSG |
| | | | GGGSGGGGSDIQMTQSPSSLSASVGDRVTITC |
| | | | RASQSISSYLNWYQQKPGKAPKLLIYAASSLQS |
| | | | GVPSRFSGSGSGTDFTLTISSLQPEDFATYYCQ |
| | | | QSYNLFTFGQGTKLEIK |
| 39 | 005038 light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQSYNLFTFGQ |
| | | | GTKLEIK |
| 40 | 005038 heavy | aa | EVQLLESGGGLVQPGGSLRLSCAASGFTFSSY |
| | chain VH | | AMSWVRQAPGKGLEWVSAISGSGGSTYYADS |
| | | | VKGRFTISRDNSKNTLYLQMNSLRAEDTAVYYC |
| | | | ARSGGYSGDHFDYWGQGTLVTVSS |
| 41 | Adecatumumab, | aa | ELQMTQSPSSLSASVGDRVTITCRTSQSISSYL |
| | light chain VL | | NWYQQKPGQPPKLLIYWASTRESGVPDRFSGS |
| | | | GSGTDFTLTISSLQPEDSATYYCQQSYDIPYTF |
| | | | GQGTKLEIK |
| 42 | Adecatumumab, | nt | gaactgcagatgacccagagcccgagcagcctgagcgcgagc |
| | light chain VL | | gtgggcgatcgcgtgaccattacctgccgcaccagccagagcatt |
| | | | agcagctatctgaactggtatcagcagaaaccgggccagccgcc |
| | | | gaaactgctgatttattgggcgagcacccgcgaaagcggcgtgc |
| | | | cggatcgctttagcggcagcggcagcggcaccgattttaccctga |
| | | | ccattagcagcctgcagccggaagatagcgcgacctattattgcc |
| | | | agcagagctatgatattccgtatacctttggccagggcaccaaact |
| | | | ggaaattaaa |
| 43 | Adecatumumab, | aa | EVQLLESGGGVVQPGRSLRLSCAASGFTFSSY |
| | heavy chain VH | | GMHWVRQAPGKGLEWVAVISYDGSNKYYADS |
| | | | VKGRFTISRDNSKNTLYLQMNSLRAEDTAVYYC |
| | | | AKDMGWGSGWRPYYYYGMDVWGQGTTVTVS |
| | | | S |
| 44 | Adecatumumab, | nt | gaagtgcagctgctggaaagcggcggcggcgtggtgcagccgg |
| | heavy chain VH | | gccgcagcctgcgcctgagctgcgcggcgagcggctttaccttta |
| | | | gcagctatggcatgcattgggtgcgccaggcgccgggcaaagg |
| | | | cctggaatgggtggcggtgattagctatgatggcagcaacaaata |
| | | | ttatgcggatagcgtgaaaggccgctttaccattagccgcgataac |
| | | | agcaaaaacaccctgtatctgcagatgaacagcctgcgcgcgga |
| | | | agataccgcggtgtattattgcgcgaaagatatgggctggggcag |
| | | | cggctggcgcccgtattattattatggcatggatgtgtggggccagg |
| | | | gcaccaccgtgaccgtgagcagc |
| 45 | 4D5MOCB | aa | DIQMTQSPSSLSASVGDRVTITCRSTKSLLHSN |
| | (alternative | | GITYLYWYQQKPGKAPKLLIYQMSNLASGVPSR |
| | name 3188), | | FSSSGSGTDFTLTISSLQPEDFATYYCAQNLEIP |
| | light chain VL | | RTFGQGTKVELK |
| 46 | 4D5MOCB, light | nt | gatattcagatgacccagagcccgagcagcctgagcgcgagcgt |
| | chain VL | | gggcgatcgcgtgaccattacctgccgcagcaccaaaagcctgc |
| | | | tgcatagcaacggcattacctatctgtattggtatcagcagaaacc |
| | | | gggcaaagcgccgaaactgctgatttatcagatgagcaacctgg |
| | | | cgagcggcgtgccgagccgctttagcagcagcggcagcggcac |
| | | | cgattttaccctgaccattagcagcctgcagccggaagattttgcga |
| | | | cctattattgcgcgcagaacctggaaattccgcgcacctttggcca |
| | | | gggcaccaaagtggaactgaaa |
| 47 | 4D5MOCB, | aa | EVQLVQSGPGLVQPGGSVRISCAASGYTFTNY |
| | heavy chain VH | | GMNWVKQAPGKGLEWMGWINTYTGESTYADS |
| | | | FKGRFTFSLDTSASAAYLQINSLRAEDTAVYYC |
| | | | ARFAIKGDYWGQGTLLTVSS |
| 48 | 4D5MOCB, | nt | gaagtgcagctggtgcagagcggcccgggcctggtgcagccgg |
| | heavy chain VH | | gcggcagcgtgcgcattagctgcgcggcgagcggctataccttta |
| | | | ccaactatggcatgaactgggtgaaacaggcgccgggcaaagg |
| | | | cctggaatggatgggctggattaacacctataccggcgaaagca |
| | | | cctatgcggatagctttaaaggccgctttacctttagcctggatacca |
| | | | gcgcgagcgcggcgtatctgcagattaacagcctgcgcgcggaa |
| | | | gataccgcggtgtattattgcgcgcgctttgcgattaaaggcgattat |
| | | | tggggccagggcaccctgctgaccgtgagcagc |
| 49 | 3-17I, light chain | aa | EIVMTQSPATLSVSPGERATLSCRASQSVSSNL |
| | VL | | AWYQQKPGQAPRLIIYGASTTASGIPARFSASG |
| | | | SGTDFTLTISSLQSEDFAVYYCQQYNNWPPAYT |
| | | | FGQGTKLEIK |
| 50 | 3-17I, light chain | nt | gaaattgtgatgacccagagcccggcgaccctgagcgtgagccc |
| | VL | | gggcgaacgcgcgaccctgagctgccgcgcgagccagagcgt |
| | | | gagcagcaacctggcgtggtatcagcagaaaccgggccaggc |
| | | | gccgcgcctgattatttatggcgcgagcaccaccgcgagcggcat |
| | | | tccggcgcgctttagcgcgagcggcagcggcaccgattttaccct |
| | | | gaccattagcagcctgcagagcgaagattttgcggtgtattattgcc |
| | | | agcagtataacaactggccgccggcgtatacctttggccagggca |
| | | | ccaaactggaaattaaa |
| 51 | 3-17I, heavy | aa | QVQLVQSGAEVKKPGSSVKVSCKASGGTFSSY |
| | chain VH | | AISWVRQAPGQGLEWMGGIIPIFGTANYAQKFQ |
| | | | GRVTITADESTSTAYMELSSLRSEDTAVYYCAR |
| | | | GLLWNYWGQGTLVTVSS |
| 52 | 3-17I, heavy | nt | caggtgcagctggtgcagagcggcgcggaagtgaaaaaaccg |
| | chain VH | | ggcagcagcgtgaaagtgagctgcaaagcgagcggcggcacc |
| | | | tttagcagctatgcgattagctgggtgcgccaggcgccgggccag |
| | | | ggcctggaatggatgggcggcattattccgatttttggcaccgcga |
| | | | actatgcgcagaaatttcagggccgcgtgaccattaccgcggatg |
| | | | aaagcaccagcaccgcgtatatggaactgagcagcctgcgcag |
| | | | cgaagataccgcggtgtattattgcgcgcgcggcctgctgtggaa |
| | | | ctattggggccagggcaccctggtgaccgtgagcagc |
| 53 | Trastuzumab, | aa | DIQMTQSPSSLSASVGDRVTITCRASQDVNTAV |
| | light chain VL | | AWYQQKPGKAPKLLIYSASFLYSGVPSRFSGS |
| | | | RSGTDFTLTISSLQPEDFATYYCQQHYTTPPTF |
| | | | GQGTKVEIK |
| 54 | Trastuzumab, | nt | gatattcagatgacccagagcccgagcagcctgagcgcgagcgt |
| | light chain VL | | gggcgatcgcgtgaccattacctgccgcgcgagccaggatgtga |
| | | | acaccgcggtggcgtggtatcagcagaaaccgggcaaagcgcc |
| | | | gaaactgctgatttatagcgcgagctttctgtatagcggcgtgccga |
| | | | gccgctttagcggcagccgcagcggcaccgattttaccctgacca |
| | | | ttagcagcctgcagccggaagattttgcgacctattattgccagca |
| | | | gcattataccaccccgccgacctttggccagggcaccaaagtgg |
| | | | aaattaaa |
| 55 | Trastuzumab, | aa | EVQLVESGGGLVQPGGSLRLSCAASGFNIKDT |
| | heavy chain VH | | YIHWVRQAPGKGLEWVARIYPTNGYTRYADSV |
| | | | KGRFTISADTSKNTAYLQMNSLRAEDTAVYYCS |
| | | | RWGGDGFYAMDYWGQGTLVTVSS |
| 56 | Trastuzumab, | nt | gaagtgcagctggtggaaagcggcggcggcctggtgcagccgg |
| | heavy chain VH | | gcggcagcctgcgcctgagctgcgcggcgagcggctttaacatta |
| | | | aagatacctatattcattgggtgcgccaggcgccgggcaaaggc |
| | | | ctggaatgggtggcgcgcatttatccgaccaacggctatacccgct |
| | | | atgcggatagcgtgaaaggccgctttaccattagcgcggatacca |
| | | | gcaaaaacaccgcgtatctgcagatgaacagcctgcgcgcgga |
| | | | agataccgcggtgtattattgcagccgctggggcggcgatggctttt |
| | | | atgcgatggattattggggccagggcaccctggtgaccgtgagca |
| | | | gc |
| 57 | Pertuzumab, | aa | DIQMTQSPSSLSASVGDRVTITCKASQDVSIGV |
| | light chain VL | | AWYQQKPGKAPKLLIYSASYRYTGVPSRFSGS |
| | | | GSGTDFTLTISSLQPEDFATYYCQQYYIYPYTFG |
| | | | QGTKVEIK |
| 58 | Pertuzumab, | nt | gatattcagatgacccagagcccgagcagcctgagcgcgagcgt |
| | light chain VL | | gggcgatcgcgtgaccattacctgcaaagcgagccaggatgtga |
| | | | gcattggcgtggcgtggtatcagcagaaaccgggcaaagcgcc |
| | | | gaaactgctgatttatagcgcgagctatcgctataccggcgtgccg |
| | | | agccgctttagcggcagcggcagcggcaccgattttaccctgacc |
| | | | attagcagcctgcagccggaagattttgcgacctattattgccagc |
| | | | agtattatatttatccgtatacctttggccagggcaccaaagtggaa |
| | | | attaaa |
| 59 | Pertuzumab, | aa | EVQLVESGGGLVQPGGSLRLSCAASGFTFTDY |
| | heavy chain VH | | TMDWVRQAPGKGLEWVADVNPNSGGSIYNQR |
| | | | FKGRFTLSVDRSKNTLYLQMNSLRAEDTAVYY |
| | | | CARNLGPSFYFDYWGQGTLVTVSS |
| 60 | Pertuzumab, | nt | gaagtgcagctggtggaaagcggcggcggcctggtgcagccgg |
| | heavy chain VH | | gcggcagcctgcgcctgagctgcgcggcgagcggctttaccttta |
| | | | ccgattataccatggattgggtgcgccaggcgccgggcaaaggc |
| | | | ctggaatgggtggcggatgtgaacccgaacagcggcggcagca |
| | | | tttataaccagcgctttaaaggccgctttaccctgagcgtggatcgc |
| | | | agcaaaaacaccctgtatctgcagatgaacagcctgcgcgcgga |
| | | | agataccgcggtgtattattgcgcgcgcaacctgggcccgagctttt |
| | | | attttgattattggggccagggcaccctggtgaccgtgagcagc |
| 61 | 2992, light chain | aa | DIQMTQSPSSLSASVGDRVTITCRASQSIRSAL |
| | VL (also known | | NWYQQKPGKAPKLLIYAASSLQSGVPSRFSGS |
| | as 2993 or 1201) | | GSGTDFTLTISSLQPEDFATYYCQQTYGYLHTF |
| | | | GQGTKLEIK |
| 62 | 2992, light chain | nt | gatattcagatgacccagagcccgagcagcctgagcgcgagcgt |
| | VL (also known | | gggcgatcgcgtgaccattacctgccgcgcgagccagagcattc |
| | as 2993 or 1201) | | gcagcgcgctgaactggtatcagcagaaaccgggcaaagcgcc |
| | | | gaaactgctgatttatgcggcgagcagcctgcagagcggcgtgc |
| | | | cgagccgctttagcggcagcggcagcggcaccgattttaccctga |
| | | | ccattagcagcctgcagccggaagattttgcgacctattattgccag |
| | | | cagacctatggctatctgcatacctttggccagggcaccaaactgg |
| | | | aaattaaa |
| 63 | 2992 heavy | aa | EVQLLESGGGLVQPGGSLRLSCAASGFDFESY |
| | chain VH (also | | AMSWVRQAPGKGLEWVSAISGSGGSTYYADS |
| | known as 1201) | | VKGRFTISRDNSKNTLYLQMNSLRAEDTAVYYC |
| | | | ARYYGGYYSAWMDYWGQGTLVTVSS |
| 64 | 2992, heavy | nt | gaagtgcagctgctggaaagcggcggcggcctggtgcagccgg |
| | chain VH (also | | gcggcagcctgcgcctgagctgcgcggcgagcggctttgattttga |
| | known as 1201) | | aagctatgcgatgagctgggtgcgccaggcgccgggcaaaggc |
| | | | ctggaatgggtgagcgcgattagcggcagcggcggcagcacct |
| | | | attatgcggatagcgtgaaaggccgctttaccattagccgcgataa |
| | | | cagcaaaaacaccctgtatctgcagatgaacagcctgcgcgcgg |
| | | | aagataccgcggtgtattattgcgcgcgctattatggcggctattata |
| | | | gcgcgtggatggattattggggccagggcaccctggtgaccgtga |
| | | | gcagc |
| 65 | Rituximab, light | aa | QIVLSQSPAILSASPGEKVTMTCRASSSVSYIH |
| | chain VL | | WFQQKPGSSPKPWIYATSNLASGVPVRFSGSG |
| | | | SGTSYSLTISRVEAEDAATYYCQQWTSNPPTF |
| | | | GGGTKLEIK |
| 66 | Rituximab, light | nt | cagattgtgctgagccagagcccggcgattctgagcgcgagccc |
| | chain VL | | gggcgaaaaagtgaccatgacctgccgcgcgagcagcagcgt |
| | | | gagctatattcattggtttcagcagaaaccgggcagcagcccgaa |
| | | | accgtggatttatgcgaccagcaacctggcgagcggcgtgccggt |
| | | | gcgctttagcggcagcggcagcggcaccagctatagcctgacca |
| | | | ttagccgcgtggaagcggaagatgcggcgacctattattgccagc |
| | | | agtggaccagcaacccgccgacctttggcggcggcaccaaact |
| | | | ggaaattaaa |
| 67 | Rituximab, | aa | QVQLQQPGAELVKPGASVKMSCKASGYTFTSY |
| | heavy chain VH | | NMHWVKQTPGRGLEWIGAIYPGNGDTSYNQK |
| | | | FKGKATLTADKSSSTAYMQLSSLTSEDSAVYYC |
| | | | ARSTYYGGDWYFNVWGAGTTVTVSA |
| 68 | Rituximab, | nt | caggtgcagctgcagcagccgggcgcggaactggtgaaaccg |
| | heavy chain VH | | ggcgcgagcgtgaaaatgagctgcaaagcgagcggctatacctt |
| | | | taccagctataacatgcattgggtgaaacagaccccgggccgcg |
| | | | gcctggaatggattggcgcgatttatccgggcaacggcgatacca |
| | | | gctataaccagaaatttaaaggcaaagcgaccctgaccgcggat |
| | | | aaaagcagcagcaccgcgtatatgcagctgagcagcctgacca |
| | | | gcgaagatagcgcggtgtattattgcgcgcgcagcacctattatgg |
| | | | cggcgattggtattttaacgtgtggggcgcgggcaccaccgtgac |
| | | | cgtgagcgcg |
| 69 | Cetuximab, light | aa | DILLTQSPVILSVSPGERVSFSCRASQSIGTNIH |
| | chain VL | | WYQQRTNGSPRLLIKYASESISGIPSRFSGSGS |
| | | | GTDFTLSINSVESEDIADYYCQQNNNWPTTFGA |
| | | | GTKLELK |
| 70 | Cetuximab, light | nt | gatattctgctgacccagagcccggtgattctgagcgtgagcccgg |
| | chain VL | | gcgaacgcgtgagctttagctgccgcgcgagccagagcattggc |
| | | | accaacattcattggtatcagcagcgcaccaacggcagcccgcg |
| | | | cctgctgattaaatatgcgagcgaaagcattagcggcattccgag |
| | | | ccgctttagcggcagcggcagcggcaccgattttaccctgagcatt |
| | | | aacagcgtggaaagcgaagatattgcggattattattgccagcag |
| | | | aacaacaactggccgaccacctttggcgcgggcaccaaactgg |
| | | | aactgaaa |
| 71 | Cetuximab, | aa | QVQLKQSGPGLVQPSQSLSITCTVSGFSLTNYG |
| | heavy chain VH | | VHWVRQSPGKGLEWLGVIWSGGNTDYNTPFT |
| | | | SRLSINKDNSKSQVFFKMNSLQSNDTAIYYCAR |
| | | | ALTYYDYEFAYWGQGTLVTVSA |
| 72 | Cetuximab, | nt | caggtgcagctgaaacagagcggcccgggcctggtgcagccga |
| | heavy chain VH | | gccagagcctgagcattacctgcaccgtgagcggctttagcctga |
| | | | ccaactatggcgtgcattgggtgcgccagagcccgggcaaaggc |
| | | | ctggaatggctgggcgtgatttggagcggcggcaacaccgattat |
| | | | aacaccccgtttaccagccgcctgagcattaacaaagataacag |
| | | | caaaagccaggtgttttttaaaatgaacagcctgcagagcaacga |
| | | | taccgcgatttattattgcgcgcgcgcgctgacctattatgattatga |
| | | | atttgcgtattggggccagggcaccctggtgaccgtgagcgcg |

TABLE C(1)
Exemplary heavy chain CDR sequences
(binding domain B1)

| Antibody | | | | | | |
|---|---|---|---|---|---|---|
| ref (VH) | SEQ | H CDR1 | SEQ | H CDR2 | SEQ | H CDR3 |
| 1132 | 73 | GFTFSSYA | 74 | IGSYGGGT | 75 | ARYVNFGMDY |
| 1150 | 73 | GFTFSSYA | 76 | IGGSSSYT | 77 | ARYYSYHMDY |
| 1140 | 73 | GFTFSSYA | 78 | ISGSGGST | 79 | ARGPVYSSVFDY |
| 1107 | 73 | GFTFSSYA | 78 | ISGSGGST | 80 | ARRVWGFDY |
| ADC- | 81 | GFTFSTYG | 82 | ISGGSSYI | 83 | ARILRG |
| 1013 | | | | | | GSGMDL |
| APX005 | 84 | GFSFSSTY | 85 | IYTGDGTN | 86 | ARPDITY |
| | | | | | | GFAINF |
| 21.4.1 | 87 | GYTFTGYY | 88 | INPDSGGT | 89 | ARDQPLGYCT |
| | | | | | | NGVCSYFDY |

TABLE C(2)
Exemplary light chain CDR sequences
(binding domain B1)

| Antibody | | | | | | |
|---|---|---|---|---|---|---|
| ref (VL) | SEQ | L CDR1 | SEQ | L CDR2 | SEQ | L CDR3 |
| 1132 (1133) | 90 | QSISSY | 91 | AAS | 92 | QQYGRNPPT |
| 1150 (1151) | 90 | QSISSY | 91 | AAS | 93 | QQYGSAPPT |
| 1140 (1135) | 90 | QSISSY | 91 | AAS | 94 | QQSYSTPYT |
| 1107 (1108) | 90 | QSISSY | 91 | AAS | 95 | QQYGVYPFT |
| ADC- | 96 | SSNIG | 97 | GNI | 98 | AAWDKS |
| 1013 | | AGYN | | | | ISGLV |
| APX005 | 99 | QSISSR | 100 | RAS | 101 | QCTGYG |
| | | | | | | ISWP |
| 21.4.1 | 102 | QGIYSW | 103 | TAS | 104 | QQANI |
| | | | | | | FPLT |

TABLE D(1)
Exemplary heavy chain CDR sequences
(binding domain B2)

| Antibody | | | | | | |
|---|---|---|---|---|---|---|
| ref (VH) | SEQ | H CDR1 | SEQ | H CDR2 | SEQ | H CDR3 |
| Solitomab | 105 | GYAFTNYW | 106 | IFPGSGNI | 107 | ARLR |
| | | | | | | NWDEPMDY |
| 005025 | 108 | SSYAMS | 109 | AISGS | 110 | GYAS |
| | | | | GGSTY | | FVGGYF |
| 005038 | 108 | SSYAMS | 109 | AISGS | 111 | SGGY |
| | | | | GGSTY | | SGDHF |
| Adecatumumab | 112 | GFTF | 113 | ISYDGSNK | 114 | AKDM |
| | | SSYG | | | | GWGSGWRP |
| | | | | | | YYYYGMDV |
| 4D5MOCB | 115 | GYTFTNYG | 116 | INTYTGES | 117 | ARFAIKGDY |
| 3-17I | 118 | GGTFSSYA | 119 | IIPIFGTA | 120 | ARGLLWNY |
| Trastuzunnab | 121 | GFNIKDTY | 122 | IYPTNGYT | 123 | SRWG |
| | | | | | | GDGFYAMDY |
| Pertuzumab | 124 | GFTFTDYT | 125 | VNPNSGGS | 126 | ARNL |
| | | | | | | GPSFYFDY |
| 2992 | 127 | GFDFESYA | 128 | ISGSGGST | 129 | ARYY |
| | | | | | | GGYYSAWMDY |
| Rituxinnab | 130 | GYTFTSYN | 131 | IYPGNGDT | 132 | ARST |
| | | | | | | YYGGDWYFNV |
| Cetuximab | 133 | GFSLTNYG | 134 | IWSGGNT | 135 | ARAL |
| | | | | | | TYYDYEFAY |

TABLE D(2)
Exemplary light chain CDR sequences
(binding domain B2)

| Antibody ref | | | | | | |
|---|---|---|---|---|---|---|
| ref (VL) | SEQ | L CDR1 | L CDR2 | | SEQ | L CDR3 |
| Solitomab | 136 | QSLLNS | 137 | WAS | 138 | QNDYSYPLT |
| | | GNQKNY | | | | |
| 005025 | 90 | QSISSY | 91 | AAS | 139 | PGSSSPY |
| 005038 | 90 | QSISSY | 91 | AAS | 140 | SYNLF |
| Adecatumumab | 90 | QSISSY | 137 | WAS | 141 | QQSYDIPYT |
| 4D5MOCB | 142 | KSLLHS | 143 | QMS | 144 | AQNLEIPRT |
| | | NGITY | | | | |
| 3-17I | 145 | QSVSSN | 146 | GAS | 147 | QQYNNWPPAYT |
| Trastuzumab | 148 | QDVNTA | 149 | SAS | 150 | QQHYTTPPT |
| Pertuzumab | 151 | QDVSIG | 149 | SAS | 152 | QQYYIYPYT |
| 2992 (2993) | 153 | QSIRSA | 91 | AAS | 154 | QQTYGYLHT |
| Rituximab | 155 | ASSSVSY | 156 | ATS | 157 | QQVVTSNPPT |
| Cetuximab | 158 | QSIGTN | 159 | YAS | 160 | QQNNNWPTT |

Mutated IgG1 antibody sequence
IgG1 LALA-sequence:
(SEQ ID NO: 161)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
Linker sequences
(SEQ ID NO: 162)
SGGGGSGGGGS
(SEQ ID NO: 163)
SGGGGSGGGGSAP
(SEQ ID NO: 164)
NFSQP
(SEQ ID NO: 165)
KRTVA
(SEQ ID NO: 166)
GGGSGGGG
(SEQ ID NO: 167)
GGGGSGGGGS
(SEQ ID NO: 168)
GGGGSGGGGSGGGGS
(SEQ ID NO: 169)
GSTSGSGKPGSGEGSTKG
(SEQ ID NO: 170)
THTCPPCPEPKSSDK
(SEQ ID NO: 171)
GGGS
(SEQ ID NO: 172)
EAAKEAAKGGGGS
(SEQ ID NO: 173)
EAAKEAAK
(SG)m, where m = 1 to 7.
IgG constant region sequences
IgG1 heavy chain constant region sequence:
[SEQ ID NO: 174]
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
IgG1 light chain constant region sequence:
[SEQ ID NO: 175]
RTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Modified IgG4 heavy chain constant region sequence:
[SEQ ID NO: 176]
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNRYTQKSLSLSLGK
Modified IgG4 heavy chain constant region sequence:
[SEQ ID NO: 177]
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
Wild type IgG4 heavy chain constant region sequence:
[SEQ ID NO: 178]
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
Reference sequence CH1 (SEQ ID NO: 179):
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSC
(wherein the bold and underlined section is part of the hinge
region, but is present in the Fab fragment)
Reference sequence CKappa (SEQ ID NO: 180):
RTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Exemplary full heavy and light chain sequences
Binding domain B1:
Heavy chain (SEQ ID NO: 181):
EVQLLESGGGLVQPGGSLRLSCAASGFTFSSYAMSWVRRAPGKGLEWVSGIGSYGGG
TYYADSVKGRFTISRDNSKNTLYLQMNSLRAEDTAVYYCARYVNFGMDYWGQGTLVTV
SSASTKGPSVFPLAPSSKSTSGGTAALGCLVKDYFPEPVTVSWNSGALTSGVATGPAV
LQSSGLYSLSSVVTVPSSSLGTQTYICNVNHKPSNTKVDKKVEPKSCDKTHTCPPCPAP
EAAGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
Light chain (SEQ ID NO: 182):
DIQMTQSPSSLSASVGDRVTITCRASQSISSYLNWYQEKPGKAPKLLIYAASSLQSGVPS
RFSGSGSGTDFTLTISSLQPEDFATYYCQQYGRNPPTFGQGTKLEIKRTVAAPSVFIFPP
SDEQLKSGTASVVCYLNNFYPREAKVQWKVDNALQSGNSQESVTEQDSKDSTYSLWS
TLTLSKADYEKHKVYACEVTHQGLSSPVTKSFNRGEC
Binding domain B2:
Heavy chain (SEQ ID NO: 183):
EVQLLEQSGAELVRPGTSVKISCKASGYAFTNYWLGWVKERPGHGLEWIGDIFPGSGN
IHYNEKFKGKATLTADKSSSTAYMQLSSLTFEDSAVYFCARLRNWDEPMDYWGQGTTV
TVSSASTKGPSVFPLAPSSKSTSGGTAALGCLVKDYFPEPVTVSWNSGALTSGVHTFP
AVLQSSGLYSLSSVVEVPSSSLGTQTYICNVNHKPSNTKVDKKVEPKSC
Light chain (SEQ ID NO: 184):
ELVMTQSPSSLTVTAGEKVTMSCKSSQSLLNSGNQKNYLTWYQRKPGQPPKLLIYWAS
TRESGVPDRFTGSGSGTDFTLTISSVQAEDLAVYYCQNDYSYPLTFGAGTKLEIKRTVA
APAVFIFPPSDEQLKSGTASVVCLLKNFYPREAKVQWKVDNALQSGNSQESVTEQDSK
DSTYSLSSTLTLSKADYEKHKWACEVTHQGLSSPVTKSFNRGEC

### EXAMPLES

### Example 1: ELISA Binding of CD40-EpCAM bsAb Towards hEpCAM

### Background and Aim

Binding was analysed with ELISA. The bispecific antibodies 1132-005025.M, 1132-005038.M, 1132-3188.M, 1132-3174.M (in Morrison format) and 1132-3174.R (in RUBY™ format) were analysed for binding towards human EpCAM.

### Material and Methods

Plates were coated with 0.5 μg/mL hEpCAM (R&D Systems #9277-EP) in PBS over night at 4° C. After washing in PBS/0.05% Tween 20 (PBST), the plates were blocked with PBS/0.2% BSA for at least 30 minutes at room temperature before being washed again. Samples serially diluted from 50 nM in PBS/0.02% BSA were then added and allowed to bind for at least 1 hour at room temperature. After washing, plates were incubated with 0.5 μg/mL biotinylated hCD40 (504-030 from Ancell) or HRP-labelled goat anti h-kappa light chain (Abd Serotec, #STAR127P), for at least 1 hour at room temperature. Dual antigen-complexed bsAb were detected with HRP-labelled streptavidin. SuperSignal Pico Luminescent was used as substrate and luminescence signals were measured using Fluostar Optima.

### Results and Conclusions

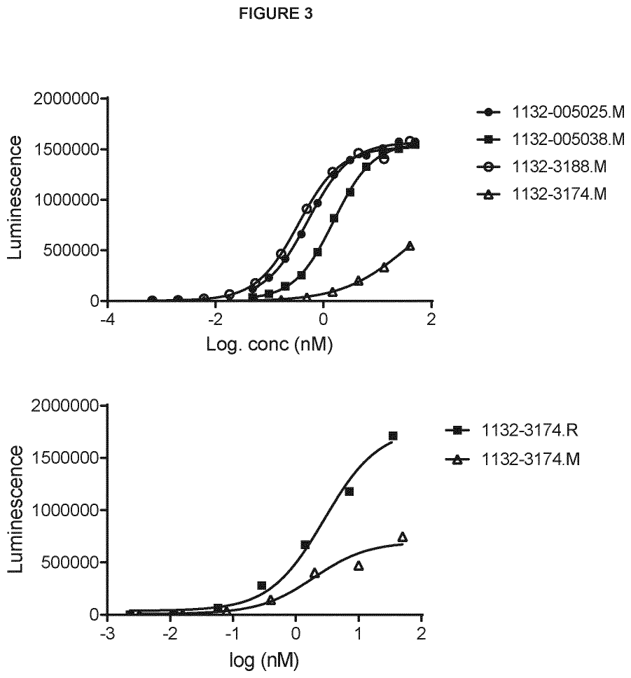

The data (shown in FIG. 3) demonstrate that 1132-005025.M, 1132-005038.M, 1132-3188.M, 1132-3174.M and 1132-3174.R bind human EpCAM.

### Example 2: Affinity Measurements of the EpCAM-Binding Domains

### Background and Aim

Binding was measured by Octet. The bispecific antibodies 1132-005025.M, 1132-005038.M, 1132-3188.M, 1132-3174.M (in Morrison format) or 1132-3174.R (in RUBY™ format) were analysed for binding towards human EpCAM.

### Material and Methods

Kinetic measurements were performed using the Octet RED96 platform (ForteBlo). The affinity evaluation was made with 3 different assays; Assay 1 with coupled bsAb and dimeric antigen EpCAM-Fc (Sino hEpCAM_Fc (0.25 mg/ml in PBS) #10694-H02H) in solution; Assay 2, with coupled bsAb and monomeric antigen EpCAM-his (R&D hEpCAM_His (500 ug/ml in PBS) #9277-EP) in solution; Assay 3 with coupled antigen (Sino hEpCAM_Fc (0.25 mg/ml in PBS) #10694-H02H) and bsAb in solution.

### Assay 1 and 2

BsAb at 1.0 or 1.5 ug/ml where coupled to anti-human Fab-CH1 2nd generation (FAB2G) biosensors (Part no #18-5125 (tray)). Antigens were serially diluted % in 1× Kinetic buffer (ForteBio) to 100 nM, 50 nM, 25 nM, 12.5 nM, 6.25 nM, 3.125 nM, 1.56 nM or 0 nM. The association was followed for 300 seconds and the dissociation in 1× Kinetic buffer for 300 seconds. Sensor tips were regenerated using 10 mM glycine, pH 1.5. Data generated were referenced by subtracting a parallel buffer blank, the baseline was aligned with the y-axis, inter-step correlation by alignment against dissociation was performed and the data were smoothed by a Savitzky-Golay filter in the data analysis software (v.9.0.0.14). The processed data were fitted using a 1:1 Langmuir binding model with X2 as a measurement of fitting accuracy.

### Assay 3

Antigen was coupled to Amine reactive Second generation sensors (Dip and Read Amine reactive Second-Generation (AR2G) Biosensors (Part no #18-5092 (tray)) at antigen concentrations of 0.4, 1.5, 0.25 or 0.5 μg/mL. BsAb (serially diluted ½ in 1× Kinetic buffer (ForteBio) with start concentrations of 20, 15, 10 or 25 nM) were analysed for binding to antigen-coupled sensors. The association was followed for 300 seconds and the dissociation in 1× Kinetic buffer for 300 seconds. Sensor tips were regenerated using 10 mM glycine, pH 2.2. Data generated were referenced by subtracting a parallel buffer blank, the baseline was aligned with the y-axis, inter-step correlation by alignment against dissociation was performed and the data were smoothed by a Savitzky-Golay filter in the data analysis software (v.9.0.0.14). The processed data were fitted using a 1:1 Langmuir binding model with X2 as a measurement of fitting accuracy.

### Results and Conclusions

All bispecific antibodies bind to human EpCAM as shown in Table 1-3 below. As expected, higher apparent affinity is measured in a bivalent setting (Assay 1 measurements). Similar affinity is observed between constructs in Morrison or RUBY™ construct.

TABLE 1
Assay 1

| bsAb | KD (M) | kon(1/Ms) | kdis(1/s) | Full X\hat{ }2 |
|---|---|---|---|---|
| 1132-005025.M | 4E−09 | 3E+05 | 1E−03 | 0.08 |
| 1132-005038.M | 6E−09 | 3E+05 | 2E−03 | 0.04 |
| 1132-3188.M | <1.0E−12 | 3E+05 | <1.0E−07 | 0.03 |
| 1132-3174.M | 5E−10 | 2E+05 | 8E−05 | 0.02 |

TABLE 2
Assay 2

| bsAb | KD (M) | kon(1/Ms) | kdis(1/s) | Full X\hat{ }2 |
|---|---|---|---|---|
| 1132-005025.M | 3E−07 | 3E+04 | 1E−02 | 0.01 |
| 1132-005038.M | 1E−06 | 8E+03 | 1E−02 | 0.01 |
| 1132-3188.M | 3E−08 | 1E+05 | 4E−03 | 0.05 |
| 1132-3174.M | 5E−07 | 2E+04 | 1E−02 | 0.01 |

TABLE 3
Assay 3

| bsAb | KD (M) | kon(1/Ms) | kdis(1/s) | Full X\hat{ }2 |
|---|---|---|---|---|
| 1132-3174.R.v9 | 8E−9 | 1E+5 | 1E−3 | 0.01 |
| 1132-3174.M | 1E−10 | 2E+5 | 2E−3 | 0.00 |

### Example 3: Binding of CD40-EpCAM Bispecific Antibodies to EpCAM-Expressing Cell Lines

### Background and Aim

1132-3174.M, 1132-005025.M, 1132-005038.M and 1132-3188.M are CD40-EpCAM bispecific antibodies in the Morrison format wherein 1132 refers to the CD40 agonist domain and 3174, 005025, 005038 and 3188 to the EpCAM-binding, tumour-targeting, domain. The antibodies have been LALA-mutated to silence Fcγ receptor binding.

The aim of this study was to assess the binding of the CD40-EpCAM bispecific antibodies to EpCAM expressed on cells.

### Materials and Methods

The human EpCAM gene was cloned into pcDNA3.1, and the vector was subsequently stably transfected into CHO cells. The tumour cell line JEG, expressing high levels of EpCAM, BxPC3 expressing low levels of EpCAM and CHO-EpCAM cells were incubated with 1 μg/ml of 1132-3174.M, 1132-005025.M, 1132-005038.M or 1132-3188.M. Binding of the antibodies was detected using fluorochrome-conjugated anti-human IgG and analysed using flow cytometry.

### Results and Conclusions

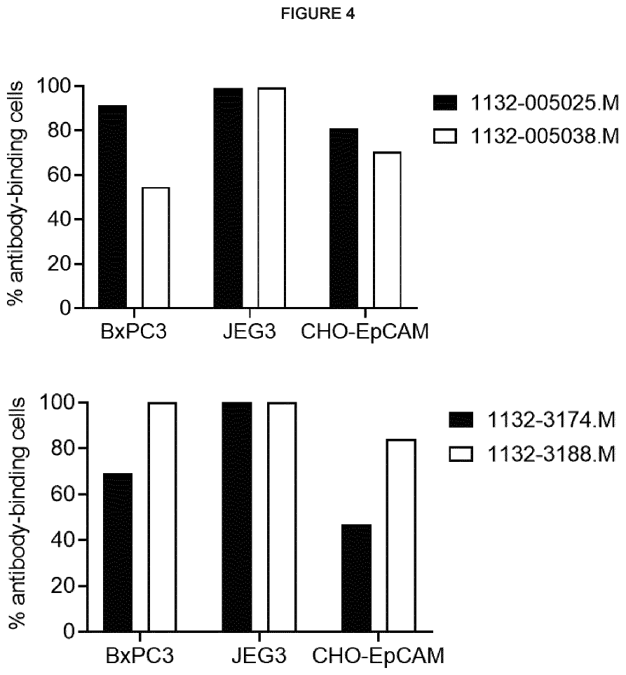

The data (shown in FIG. 4) demonstrate that all tested CD40-EpCAM bispecific antibodies bind to EpCAM expressed on all tested cell lines.

### Example 4: Binding of the CD40-EpCAM Bispecific Antibody 1132-3174.R in RUBY™ Format to EpCAM-Transfected CHO Cells

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to assess the binding of 1132-3174.R to CHO cells transfected with human EpCAM.

### Materials and Methods

The CD40-EpCAM bispecific antibody 1132-3174.R and the CD40 monospecific antibody 1132.m2 were biotinylated using EZ-Link Sulfo-NHS-LC-Biotin (ThermoFisher #A39257). The human EpCAM gene was cloned into pcDNA3.1, and the vector was subsequently stably transfected into CHO cells. Control CHO cells were stably transfected with and empty pcDNA3.1 vector. CHO cells were incubated with titrated concentrations of biotinylated 1132-3174.R or 1132.m2. Binding of biotinylated antibodies was detected with fluorochrome-conjugated streptavidin and analysed using flow cytometry.

### Results and Conclusions

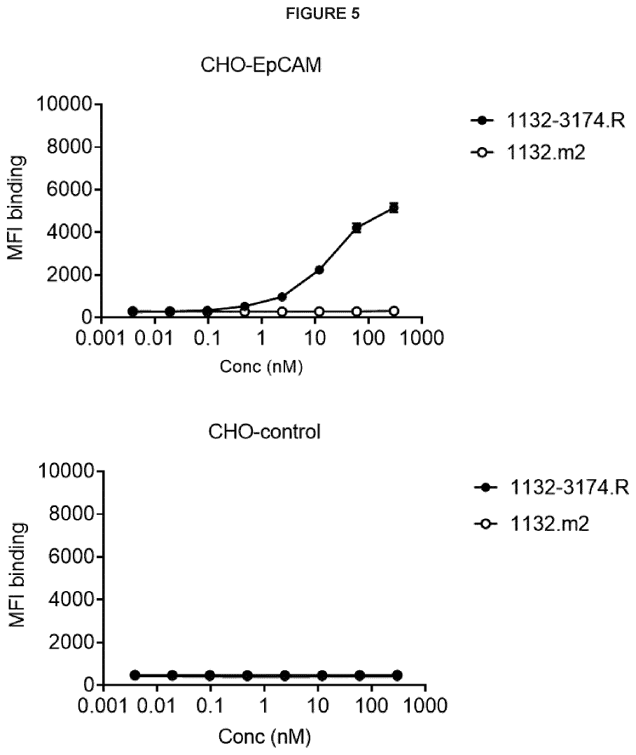

The data (shown in FIG. 5) demonstrate that 1132-3174.R binds to EpCAM-transfected but not control CHO cells. 1132.m2 does not bind to either cell line.

### Example 5: Binding of the CD40-EpCAM Bispecific Antibody 1132-3174.R in RUBY™ Format to EpCAM-Expressing Tumour Cell Lines

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to assess the binding of 1132-3174.R to tumour cell lines of different origin expressing varying levels of EpCAM.

### Materials and Methods

The CD40-EpCAM bispecific antibody 1132-3174.R and the CD40 monospecific antibody 1132.m2 were biotinylated using EZ-Link Sulfo-NHS-LC-Biotin (ThermoFisher #A39257). The tumour cell lines HT29, JEG and JAR expressing high levels of EpCAM (+++), and BxPC3 expressing low levels of EpCAM (++) were incubated with titrated concentrations of biotinylated 1132-3174.R or 1132.m2. Binding of biotinylated antibodies was detected with fluorochrome-conjugated streptavidin and analysed using flow cytometry.

### Results and Conclusions

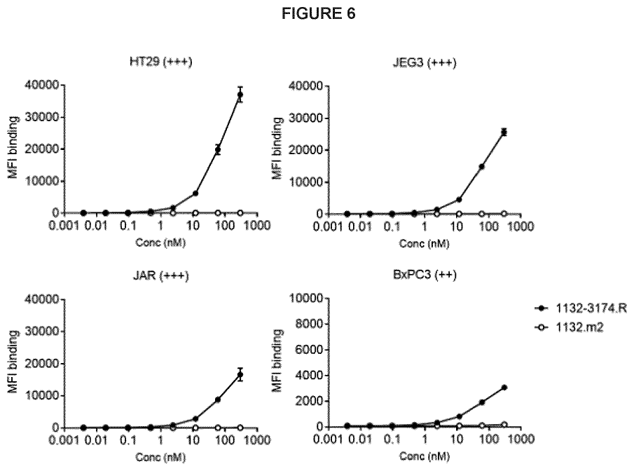

The data (shown in FIG. 6) demonstrate that 1132-3174.R but not 1132.m2 binds to all tested EpCAM+ tumour cell lines.

### Example 6: Binding of the CD40-EpCAM Bispecific Antibody 1132-3174.R in RUBY™ Format to Human Peripheral Blood Mononuclear Cells

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to assess the binding of 1132-3174.R to CD40+ and CD40− cell populations among human peripheral blood mononuclear cells.

### Materials and Methods

The CD40-EpCAM bispecific antibody 1132-3174.R and the CD40 monospecific antibody 1132.m2 were biotinylated using EZ-Link Sulfo-NHS-LC-Biotin (ThermoFisher #A39257). Human peripheral blood mononuclear cells (PBMC) were incubated with titrated concentrations of biotinylated 1132-3174.R or 1132.m2 and fluorochrome-conjugated antibodies directed against the B cell marker CD19, T cell marker CD3, NK cell marker CD56 and monocyte marker CD14. Binding of biotinylated antibodies was detected with fluorochrome-conjugated streptavidin and analysed using flow cytometry.

### Results and Conclusions

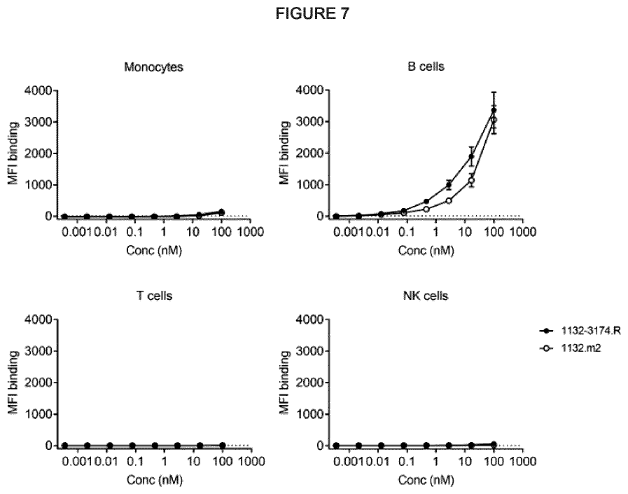

The data (shown in FIG. 7) demonstrate that both 1132-3174.R and 1132.m2 bind specifically to CD40+ cell populations among PBMC, where B cells have a relatively high CD40 expression and monocytes have a low CD40 expression. 1132-3174.R and 1132.m2 do not bind to T cells or NK cells, which do not express CD40.

### Example 7: Agonistic Effect of the CD40-EpCAM Bispecific Antibodies in a B Cell Proliferation Assay

### Background and Aim

1132-3174.M, 1132-005038.M, 1132-005025.M and 1132-3188.M are CD40-EpCAM bispecific antibodies in the Morrison format wherein 1132 refers to the CD40 agonist domain and 3174, 005038, 005025 and 3188 to the EpCAM-binding, tumour-targeting, domain. The antibodies have been LALA-mutated to silence Fcγ receptor binding.

The aim of this study was to assess the effect of the CD40-EpCAM bispecific antibodies on B cell activation in vitro in the presence or absence of EpCAM. CD40 crosslinking will be mediated by simultaneous binding of CD40, expressed on B cells, and EpCAM expressed on Chinese hamster ovarian (CHO) cells.

### Materials and Methods

The agonistic effect of 1132-3174.M, 1132-005038.M, 1132-005025.M and 1132-3188.M was assessed in a B cell assay, based on primary human B cells. Briefly, B cells were isolated from human peripheral blood mononuclear cells by MACS according to the manufacturer's protocol (Miltenyi Biotec #130-091-151). Human EpCAM transfected CHO cells, or CHO cells transfected with an empty vector were UV irradiated and seeded in tissue culture treated 96 well flat bottom plates (Eppendorf). B cells were cocultured with the CHO cells in the presence of IL-4 (10 ng/ml, Gibco #PHC0045) and titrated concentrations of 1132-3174.M, 1132-005038.M, 1132-005025.M or 1132-3188.M. After 2 days, B cells were harvested and expression level of the activation marker CD86 was analysed by FACS.

### Results and Conclusions

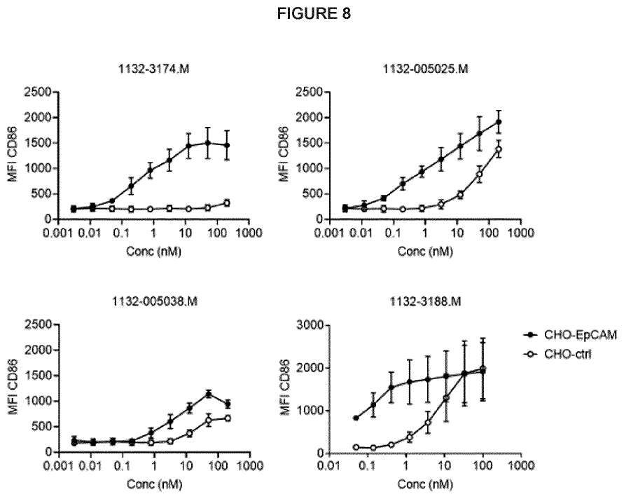

The data (shown in FIG. 8) demonstrate that all tested CD40-EpCAM bispecific antibodies induce upregulation of CD86 on B cells in the presence of EpCAM. In contrast to 1132-005038.M, 1132-005025.M and 1132-3188.M, no B cell activation in the absence of EpCAM is observed with 1132-3174.M.

### Example 8: Agonistic Effect of the CD40-EpCAM Bispecific Antibody 1132-3174.R in a B Cell Proliferation Assay

### Background and Aim

1132-3174.R is a CD40-EpCAM bispecific antibody in the RUBY™ format wherein 1132 refers to the CD40 agonist domain and 3174 to the EpCAM-binding, tumour-targeting, domain. The antibody has been LALA-mutated to silence Fcγ receptor binding.

The aim of this study was to assess the effect of 1132-3174.R on B cell activation in vitro in the presence or absence of EpCAM. CD40 crosslinking will be mediated by simultaneous binding of CD40, expressed on B cells, and EpCAM expressed on Chinese hamster ovarian (CHO) cells.

### Materials and Methods

The agonistic effect of 1132-3174.R was assessed in a B cell assay, based on primary human B cells. Briefly, B cells were isolated from human peripheral blood mononuclear cells by MACS according to the manufacturer's protocol (Miltenyi Biotec #130-091-151). Human EpCAM transfected CHO cells, or CHO cells transfected with an empty vector were UV irradiated and seeded in tissue culture treated 96 well flat bottom plates (Eppendorf). B cells were cocultured with the CHO cells in the presence of IL-4 (10 ng/ml, Gibco #PHC0045) and titrated concentrations of 1132-3174.R. After 2 days, B cells were harvested and expression level of the activation marker CD86 was analysed by FRCS.

### Results and Conclusions

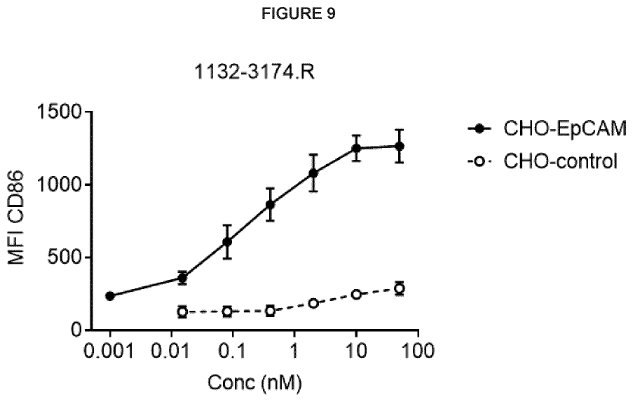

The data (shown in FIG. 9) demonstrates that 1132-3174.R induces upregulation of CD86 on B cells in the presence of EpCAM, with minimal B cell activation seen in the absence of EpCAM.

### Example 9: Agonistic Effect of the CD40-5T4 Bispecific Antibody 1132-1210.M in a B Cell Proliferation Assay

### Background and Aim

1132-1210.M is a CD40-5T4 bispecific antibody in the Morrison format wherein 1132 refers to its CD40 agonist domain and 1210 to its 5T4-binding, tumour-targeting, domain. The antibody has been LALA-mutated to silence Fcγ receptor binding.

The aim of this study was to assess the effect of 1132-1210.M on B cell proliferation in vitro in the presence or absence of 5T4. CD40 crosslinking will be mediated by simultaneous binding of CD40, expressed on B cells, and 5T4 antigen, coated to the plastic.

### Materials and Methods

The agonistic effect of 1132-1210.M was assessed in a B cell assay, based on primary human B cells. Briefly, B cells were isolated from human peripheral blood mononuclear cells by MACS according to the manufacturer's protocol (Miltenyi Biotec #130-091-151). 5T4-Fc was coated to the plastic of sterile white 96 well flat-bottom plates (Greiner #655074), followed by blocking with culture media containing 10% FBS. Control un-coated wells were blocked with culture media containing 10% FBS. B cells were cultured for 2 days in the presence of IL-4 (10 ng/ml, Gibco #PHC0045) and titrated concentrations of 1132-1210.M, 1132.m2 (a LALA-mutated agonistic monoclonal CD40 antibody) or 1188-1210.M (a LALA-mutated isotype control-5T4 bispecific antibody). Proliferation readout was performed using CellTiter-Glo Luminescent cell viability assay (Promega #G7571).

To be able to pool results from different donors, data was normalized to the mean of the culture media (R10) control.

### Results and Conclusions

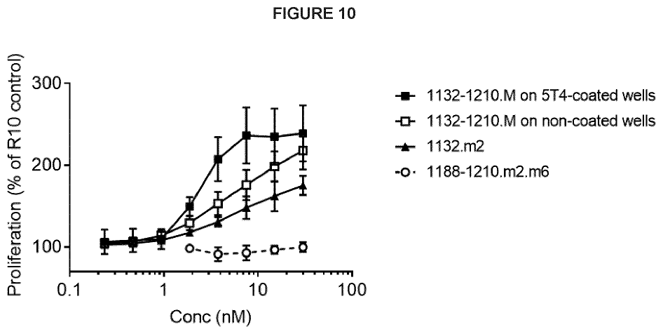

The data (shown in FIG. 10) demonstrate that 1132-1210.M induces B cell proliferation in the presence of 5T4, however it also induces B cell proliferation in the absence of 5T4, although not to the same degree. The LALA-mutated CD40 mAb 1132.m2 also induces some B cell proliferation in this assay.

### Example 10: Agonistic Effect of the CD40-EpCAM Bispecific Antibodies 1132-3174.M and 1132-3174.R in a Dendritic Cell Activation Assay

### Background and Aim

1132-3174.M is a CD40-EpCAM bispecific antibody in the Morrison format wherein 1132 refers to the CD40 agonist domain and 3174 to the EpCAM-binding, tumour-targeting, domain. 1132-3174.R has the same CD40 and EpCAM-binding domains but is produced in the RUBY™ format. The antibodies have been LALA-mutated to silence Fcγ receptor binding.

The aim of this study was to assess the effect of 1132-3174.M and 1132-3174.R on dendritic cell (DC) activation in vitro in the presence or absence of EpCAM. CD40 crosslinking will be mediated by simultaneous binding of CD40, expressed on DCs, and EpCAM expressed on Chinese hamster ovarian (CHO) cells.

### Materials and Methods

The agonistic effect of 1132-3174.M and 1132-3174.R was assessed in a DC activation assay, based on DCs derived from primary human monocytes. Briefly, monocytes were isolated from human peripheral blood mononuclear cells by MACS according to the manufacturer's protocol (Miltenyi Biotec #130-050-201). DCs were generated by culturing monocytes for 7 days in the presence of GM-CSF (150 ng/ml, Gibco) and IL-4 (50 ng/ml, Gibco). Human EpCAM-transfected CHO cells, or CHO cells transfected with an empty vector were UV irradiated and seeded in tissue culture treated 96 well flat bottom plates (Eppendorf). DCs were cocultured with the CHO cells in the presence of GM-CSF, IL-4 and titrated concentrations of 1132-3174.M or 1132-3174.R. After 2 days, DCs were harvested and expression of HLA-DR and costimulatory molecule CD86 on CD14− CD1a+ DCs was analysed by FACS.

### Results and Conclusions

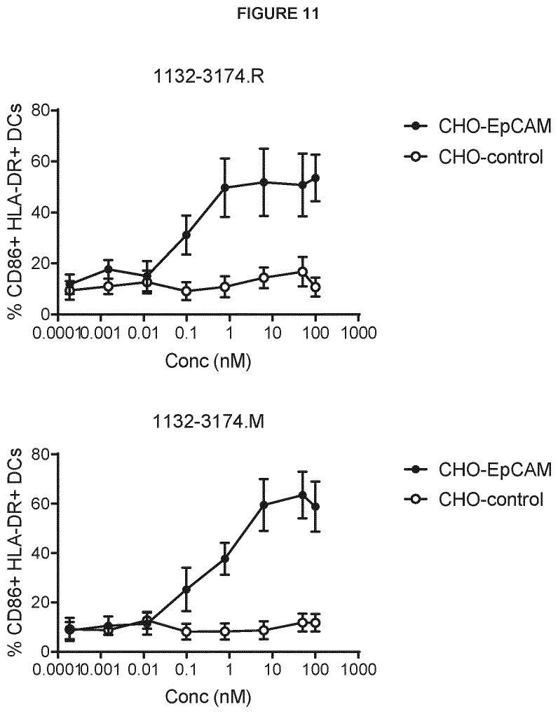

The data (shown in FIG. 11) demonstrate that both 1132-3174.M and 1132-3174.R induce DC activation, measured as increased expression of CD86 and HLA-DR, on DCs in the presence of EpCAM, without inducing background activation in the absence of EpCAM.

### Example 11: Agonistic Effect of the CD40-EpCAM Bispecific Antibodies 1132-3174.M and 1132-3174.R on IL-12 Production by Dendritic Cells

### Background and Aim

1132-3174.M is a CD40-EpCAM bispecific antibody in the Morrison format wherein 1132 refers to the CD40 agonist domain and 3174 to the EpCAM-binding, tumour-targeting, domain. 1132-3174.R has the same CD40 and EpCAM-binding domains but is produced in the RUBY™ format. The antibodies have been LALA-mutated to silence Fcγ receptor binding.

The aim of this study was to assess the effect of 1132-3174.M and 1132-3174.R on IL-12 production by dendritic cells (DC) in vitro in the presence or absence of EpCAM. CD40 crosslinking will be mediated by simultaneous binding of CD40, expressed on DCs, and EpCAM expressed on Chinese hamster ovarian (CHO) cells.

### Materials and Methods

The agonistic effect of 1132-3174.M and 1132-3174.R was assessed in a DC activation assay, based on DCs derived from primary human monocytes. Briefly, monocytes were isolated from human peripheral blood mononuclear cells by MACS according to the manufacturer's protocol (Miltenyi Biotec #130-050-201). DCs were generated by culturing monocytes for 7 days in the presence of GM-CSF (150 ng/ml, Gibco) and IL-4 (50 ng/ml, Gibco). Human EpCAM-transfected CHO cells, or CHO cells transfected with an empty vector were UV irradiated and seeded in tissue culture treated 96 well flat bottom plates (Eppendorf). DCs were cocultured with the CHO cells in the presence of GM-CSF, IL-4 and titrated concentrations of 1132-3174.M or 1132-3174.R. After 2 days, supernatants were collected and IL-12p40 content was analysed by ELISA (Biolegend #430701).

### Results and Conclusions

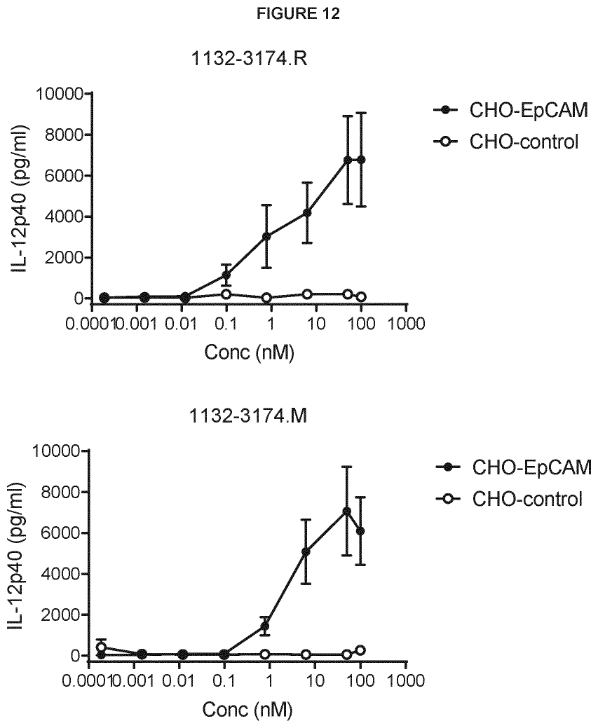

The data (as shown in FIG. 12) demonstrate that both 1132-3174.M and 1132-3174.R induce IL-12p40 release by DCs in the presence of EpCAM, without inducing background IL-12p40 release in the absence of EpCAM.

### Example 12: Effect of the CD40-EpCAM Bispecific Antibodies 1132-3174.M and 1132-3174.R on Co-Localization (Such as, Internalization) of EpCAM+ Tumour Cell Debris in a CD40-Expressing Cell Line

### Background and Aim

1132-3174.M is a CD40-EpCAM bispecific antibody in the Morrison format wherein 1132 refers to the CD40 agonist domain and 3174 to the EpCAM-binding, tumour-targeting, domain. 1132-3174.R has the same CD40 and EpCAM-binding domains but is produced in the RUBY™ format. The antibodies have been LALA-mutated to silence Fcγ receptor binding.

The aim of this study was to assess the effect of 1132-3174.M and 1132-3174.R on co-localization (such as, internalization) of cell debris from an EpCAM+ tumour cell line into CD40+ cells.

### Materials and Methods

The human EpCAM+ tumour cell line JAR was stained with the fluorescent membrane dye PKH26 (Sigma-Aldrich) followed by heat shock at 45° C. for 10 min to induce cell death. Heat-shocked tumour cells were incubated at 37° C. overnight, spun down and supernatant containing tumour cell debris was collected.

CD40+ Raji cells were labelled with the nuclear stain Hoechst 33342 (0.045 μg/ml, Thermo Fisher). Raji cells were cultured with tumour cell debris and titrated concentrations of 1132-3174.M, 1132-3174.R or the monoclonal CD40 antibody 1132.m2. Cells were imaged every hour using the live cell imaging system Cytation5 (BioTek). Images were analysed and the number of tumour debris localized in Raji cells was quantified using Gen5 software (BioTek).

### Results and Conclusions

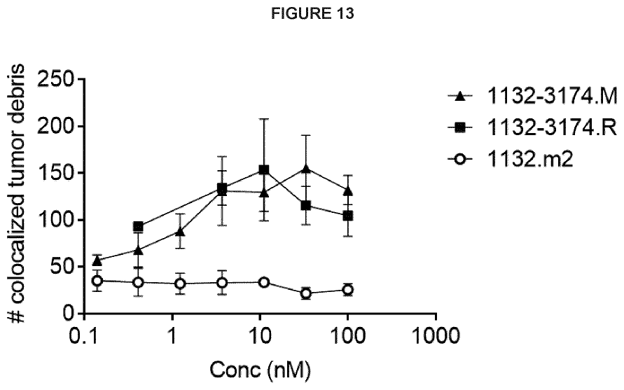

The data (shown in FIG. 13) demonstrate that both 1132-3174.M and 1132-3174.R mediate increased localization of EpCAM+ tumour cell debris in CD40+ cells, whereas the CD40 monoclonal antibody does not.

### Example 13: Agonistic Effect of the CD40 Monoclonal Antibodies 1132/1133, 1140/1135 and 1150/1151

### Background and Aim

The aim of this study was to evaluate the monoclonal human CD40 agonistic IgG1 antibodies 1132/1133, 1140/1135 and 1150/1151 with respect to their capability to activate antigen-presenting cells such as dendritic cells and B cells in vivo in human CD40 transgenic (hCD40tg) mice.

### Materials and Methods

Female hCD40tg mice of 9-12 weeks of age were administered 100 μg of 1132/1133, 1140/1135 or 1150/1151 i.p. at the start of the experiment and once more, three days later. A group of control IgG-treated mice was also included. On day 4, one day following the final dosing, the mice were sacrificed and the spleens collected. The spleens were mashed through cell strainers to obtain single cell suspensions and the cells were subsequently Fc blocked and stained with an antibody cocktail containing fluorescently-labelled anti-mouse antibodies for CD11b, CD11c, CD19, CD45, CD80, CD86 and MHCII. This was done in order to determine the activation of CD11c⁺ MHCII⁺ dendritic cells and CD19⁺ MHCII⁺ B cells, based on the levels of the co-stimulatory markers CD80 and CD86, by flow cytometry. The cells were also stained with Fixable Viability Stain 450 to assess the cell viability.

### Results and Conclusions

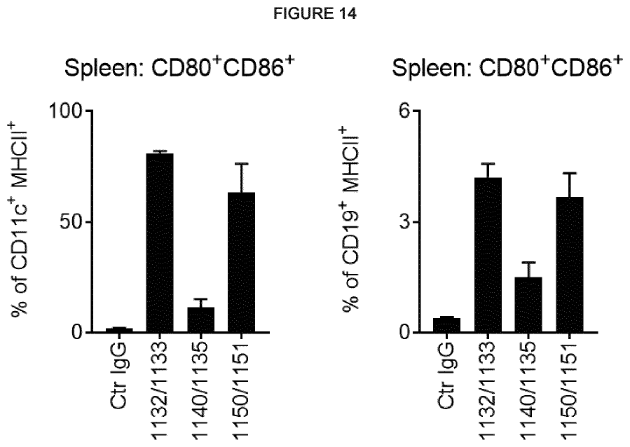

The data (shown in FIG. 14) demonstrate that antibody clones 1132/1133 and 1150/1151 display a very potent activation of splenic dendritic cells and B cells, while clone 1140/1135 shows very modest effects on the activation of these cell populations.

### Example 14: Localization of the CD40-EpCAM Bispecific Antibody 1132-3174.R in RUBY™ Format to EpCAM-Expressing Tumours

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to evaluate the tumour localization of 1132-3174.R administered to C57Bl/6 mice inoculated with murine MB49 tumours transfected with human EpCAM (MB49-hEpCAM), compared to CD40 monospecific 1132.m2.

### Materials and Methods

Female C57Bl/6 mice, either human CD40 transgenic (hCD40tg) or non-hCD40tg mice of 13-14 weeks of age, were inoculated with 2.5×10⁵ MB49-hEpCAM cells s.c. in the right flank. On day 10 after inoculation, the mice were administered i.p. with 333 μg of 1132-3174.R or 200 μg of LALA-mutated CD40 monospecific antibody, 1132.m2. A group of vehicle-treated mice was also included. On day 11, one day following the final dosing of the mice, the mice were sacrificed and the tumours collected. The tumours were cut into pieces, enzymatically digested with DNase and liberase, and mashed through cell strainers in order to obtain single cell suspensions. The cells were Fc blocked and stained with APC eFluor780-conjugated anti-mouse CD45 and PE-conjugated anti-human IgG antibody to determine the degree of antibody localization to the tumour tissue by flow cytometry. The cells were also stained with Fixable Viability Stain 450 to assess the cell viability.

### Results and Conclusions

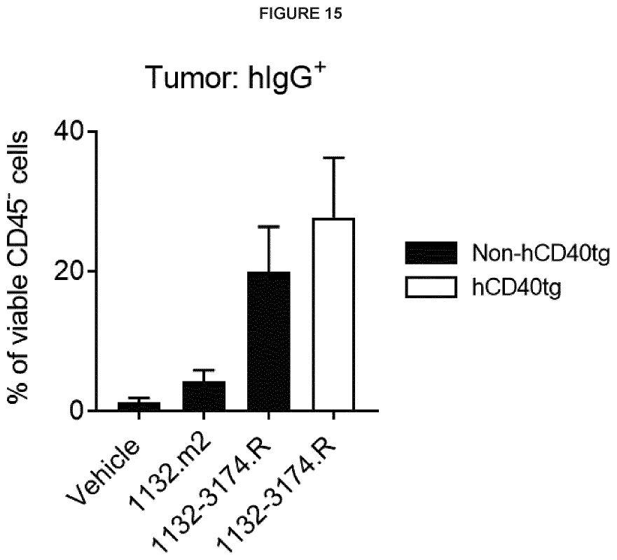

The data (shown in FIG. 15) demonstrate that, compared to 1132.m2, 1132-3174.R significantly more potently locates to MB49-hEpCAM tumours in non-hCD40tg mice. A similar degree of localization of 1132-3174.R could be observed in hCD40tg mice.

### Example 15: Localization of the CD40-5T4 Bispecific Antibody 1132-1210.M in Morrison Format to 5T4-Expressing Tumours

### Background and Aim

1132-1210.M is a CD40-5T4 bispecific antibody in the Morrison format wherein 1132 refers to its CD40 agonist domain and 1210 to its 5T4-binding, tumour-targeting, domain. The antibody has been LALA-mutated to silence Fcγ receptor binding.

The aim of this study was to evaluate the tumour localization of 1132-1210.M administered to human CD40 transgenic (hCD40tg) mice inoculated with either murine B16 tumours transfected with human 5T4 (B16-h5T4) or B16.F10 (h5T4 negative) tumours.

### Materials and Methods

Female hCD40tg mice of 13-15 weeks of age were inoculated with either 1×10⁵ B16.F10 or B16-h5T4 cells s.c. in the right flank. On days 16 and 19 after inoculation, the mice were administered i.p. with 100 μg of either wildtype or LALA-mutated CD40 monospecific antibody, 1132 or 1132.m2, respectively. Alternatively, the mice received a high dose, 485 μg, of either 1132-1210.M or 1188-1210.M, an isotype-5T4 bispecific antibody. A group of vehicle-treated mice was also included. On day 20, one day following the final dosing of the mice, the mice were sacrificed and the tumours collected. The tumours were cut into pieces, enzymatically digested with DNase and liberase and mashed through cell strainers in order to obtain single cell suspensions. The cells were Fc blocked and stained with APC eFluor780-conjugated anti-mouse CD45 and PE-conjugated anti-human IgG antibody to determine the degree of antibody localization to the tumour tissue by flow cytometry. The cells were also stained with Fixable Viability Stain 450 to assess the cell viability.

### Results and Conclusions

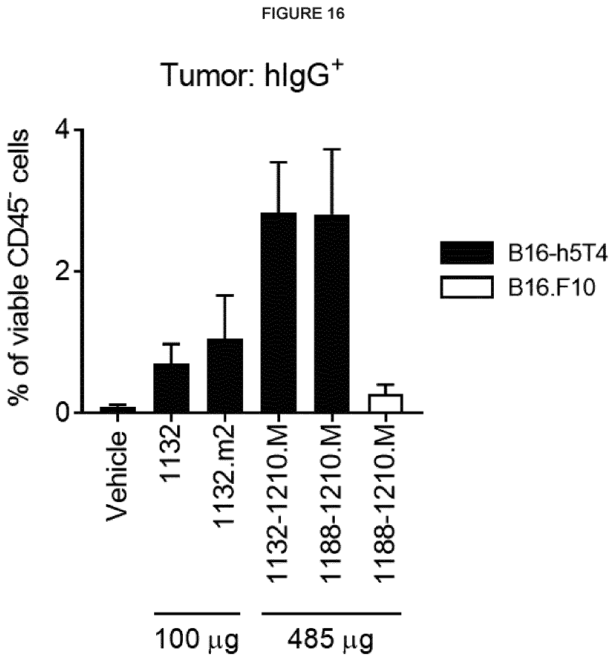

The data (shown in FIG. 16) demonstrate that, compared to 1132 or 1132.m2, 1132-1210.M as well as 1188-1210.M significantly more potently locate to B16-h5T4 tumours. In B16.F10 tumours lacking the target tumour antigen 5T4, the tumour-localizing effect of 1188-1210.M is almost completely diminished. This suggests that 1132-1210.M potently locates to 5T4-expressing tumours and that this is mediated via binding of 1210 to 5T4.

### Example 16: Anti-Tumour Effect of the CD40-EpCAM Bispecific Antibody 1132-3174.M in Morrison Format

### Background and Aim

1132-3174.M is a CD40-EpCAM bispecific antibody in Morrison format wherein 1132 refers to its CD40 agonist domain and 3174 to its EpCAM-binding, tumour-targeting domain. The antibody has been LALA-mutated to silence Fcγ receptor binding.

The aim of this study was to evaluate the anti-tumour effect of 1132-3174.M in human CD40 transgenic (hCD40tg) mice inoculated with murine MB49 tumours transfected with human EpCAM (MB49-hEpCAM) or MB49-wt (hEpCAM negative) tumours.

### Materials and Methods

Female hCD40tg mice of 12-15 weeks of age were inoculated with either 2.5×10⁵ MB49-wt or MB49-hEpCAM cells s.c. in the right flank. On days 7, 10 and 13 after inoculation, the mice were administered i.p. with 100 μg of wildtype CD40 monospecific antibody, 1132, or 250 μg of the LALA-mutated equivalent, 1132.m2. Alternatively, the mice received 333 μg of 1132-3174.M. A group of vehicle-treated mice was also included. The tumours were frequently measured with a caliper in width (w), length (l) and height (h) and the tumour volume was calculated using the formula: (w/2×l/2×h/2×π×(4/3)).

### Results and Conclusions

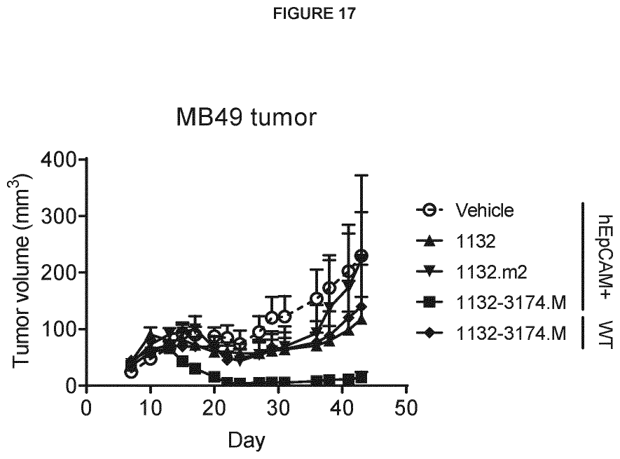

The data (shown in FIG. 17) demonstrate that treatment with 1132-3174.M significantly reduces the tumour volume compared to vehicle-treated mice, as well as mice treated with 1132. Additionally, in mice bearing MB49-wt tumours administered the same dosage of 1132-3174.M, the anti-tumour effect of 1132-3174.M is almost completely diminished. Thus, 1132-3174.M has a potent, EpCAM-dependent anti-tumour effect in the MB49 tumour model.

### Example 17: Anti-Tumour Effect of the CD40-EpCAM Bispecific Antibody 1132-3174.R in RUBY™ Format

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to evaluate the anti-tumour effect of 1132-3174.R in human CD40 transgenic (hCD40tg) mice inoculated with murine MB49 tumours transfected with human EpCAM (MB49-hEpCAM) or MB49-wt (hEpCAM negative) tumours.

### Materials and Methods

Female hCD40tg mice of 13-16 weeks of age were inoculated with either 2.5×10⁵ MB49-wt or MB49-hEpCAM cells s.c. in the right flank. On days 10, 13 and 16 after inoculation, the mice were administered i.p. with 100 μg of wildtype CD40 monospecific antibody, 1132, or 250 μg of the LALA-mutated equivalent, 1132.m2. Alternatively, the mice received 417 μg of 1132-3174.R. A group of vehicle-treated mice was also included. The tumours were frequently measured with a caliper in width (w), length (l) and height (h) and the tumour volume was calculated using the formula: (w/2×l/2×h/2×π×(4/3)).

In an alternative experimental set-up, hCD40tg mice were inoculated with MB49-wt or MB49-hEpCAM cells s.c. as previously and, instead, mice were administered i.p. with 100 μg 1132, 100 μg 1132.m2 or 167 μg (dose of molecular mass equivalence to the monospecific antibodies) or 417 μg (dose 2.5 fold higher in terms of molecular mass, compared to monospecific antibodies) 1132-3174.R on days 10, 13 and 16 after inoculation. A group of vehicle-treated mice was also included. Tumors were frequently measured as previously.

### Results and Conclusions

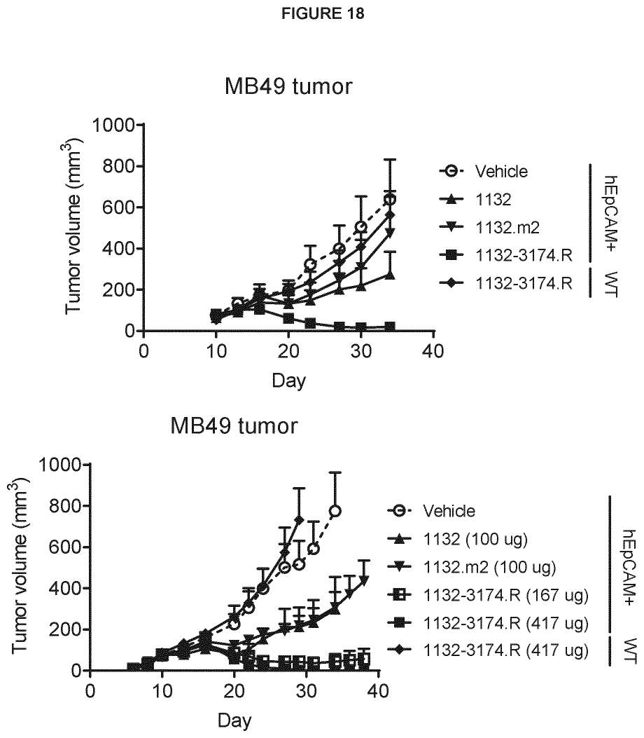

The data (shown in FIG. 18) demonstrate that treatment with 1132-3174.R significantly reduces the tumour volume compared to vehicle-treated mice, as well as mice treated with 1132. Additionally, in mice bearing MB49-wt tumours administered the same dosage of 1132-3174.R, the anti-tumour effect of 1132-3174.R is almost completely diminished. Thus, 1132-3174.R has a potent, EpCAM-dependent anti-tumour effect in the MB49 tumour model.

### Example 18: Immunological Memory Induced by the CD40-EpCAM Bispecific Antibody 1132-3174.R in RUBY™ Format

### Background and Aim

Immunomodulators are considered to induce long-term curative responses against cancer as they induce immunological memory. The aim of this study was to demonstrate such immunological memory induced in hCD40tg mice in which 1132-3174.R treatment had resulted in complete tumour regression. These mice were rechallenged with the same MB49-hEpCAM tumours, or with MB49 tumours lacking expression of hEpCAM.

### Materials and Methods

Naïve female hCD40tg mice of 14 weeks of age, or hCD40tg mice which had previously been inoculated with MB49-hEpCAM tumours and cured of the tumours following treatment with 1132-3174.R, were used in the study. All mice were inoculated s.c. (subcutaneously) with tumour cells on both sides of the flank; 2.5×10⁵ MB49-hEpCAM cells on the left and 2.5×10⁵ MB49-wt cells on the right. The tumours were frequently measured with a caliper in width (w), length (l) and height (h) and the tumour volume was calculated using the formula: (w/2×l/2×h/2×π×(4/3)). No treatments were administered during the study.

### Results and Conclusions

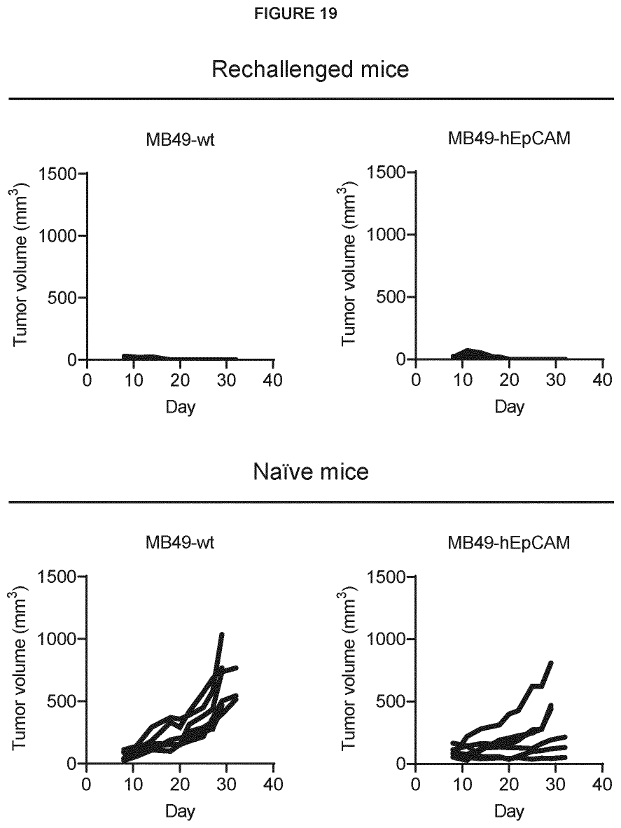

The data (shown in FIG. 19) demonstrate that in rechallenged mice, neither MB49-wt nor MB49-hEpCAM tumours display any detectable growth, while in naïve mice both MB49-wt and MB49-hEpCAM tumours grow equally well. This suggests that the rechallenged mice have acquired immunological memory to the MB49 tumour following treatment with 1132-3174.R, and that this immunological memory is not specifically directed to EpCAM.

### Example 19: In Vivo Safety of the CD40-EpCAM Bispecific Antibody 1132-3174.R in RUBY™ Format

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to evaluate the safety profile of 1132.3174.R, compared to monospecific anti-CD40 antibodies. The parameters evaluated were spleen enlargement and IL-6 cytokine release.

### Materials and Methods

Female hCD40tg mice of 10-14 weeks of age were inoculated with MB49-hEpCAM cells s.c. (subcutaneously) in the right flank. On days 10, 13 and 16 after inoculation, the mice were administered i.p. (intraperitoneally) with two dose levels of wildtype CD40 monospecific antibody, 1132, or the LALA-mutated equivalent, 1132.m2, which were administered at either 100 μg or 250 μg. Alternatively, the mice received 167 μg or 417 μg 1132-3174.R, dose levels of molecular mass equivalence to the monospecific antibodies. Control groups included mice treated with 100 μg of a reference anti-CD40 antibody, or vehicle-treated mice.

Blood was collected via vena saphena 4 hrs after the therapy treatments on days 10 and 13 and plasma was obtained from the samples. The samples were analysed for IL-6 using the IL-6 High Sensitivity ELISA Kit according to the manufacturer's protocol (Invitrogen #BM S603HS). On day 20, 4 days after the final therapy treatment, mice were sacrificed and spleens were weighed.

### Results and Conclusions

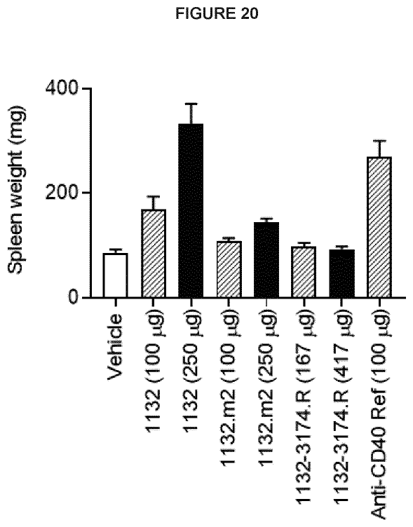

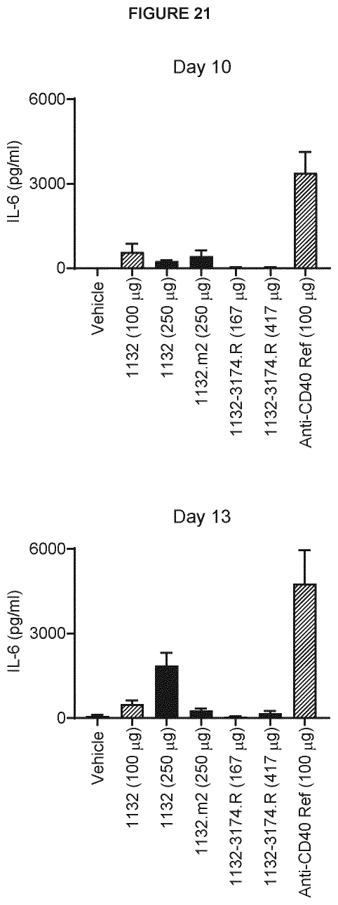

The data (shown in FIGS. 20 and 21) demonstrate that administration of both doses of 1132 results in enlarged spleens and increased IL-6 cytokine release, compared to vehicle-treated controls. The spleen enlargement, and to some extent the cytokine release, is less pronounced in mice treated with 1132.m2. Neither of the two doses of 1132-3174.R result in any spleen enlargement or IL-6 release, when compared to vehicle-treated controls.

Thus, in comparison to 1132, and the anti-CD40 reference antibody, these data clearly demonstrate an improved safety profile of 1132.3174.R, in terms of spleen enlargement and IL-6 cytokine release.

### Example 20: Immunological Memory Induced In Vivo by the CD40-EpCAM Bispecific Antibody 1132-3174.R

### Background and Aim

Immunomodulators are considered to induce long-term curative responses against cancer as they induce immunological memory. The aim of this study was to demonstrate such immunological memory induced in hCD40tg mice in which 1132-3174.R treatment had resulted in complete regression of MB49-hEpCAM tumours. These mice were rechallenged with MB49-wt tumours lacking expression of hEpCAM, or with irrelevant Panc02 tumours.

### Materials and Methods

Naïve female hCD40tg mice of 11 weeks of age, or hCD40tg mice which had previously been inoculated with MB49-hEpCAM tumours and cured of the tumours following treatment with 1132-3174.R, were used in the study. All mice were inoculated s.c. with tumour cells on both sides of the flank; 2.5×10⁵ MB49-wt cells on the left and 2.5×10⁵ Panc02 cells on the right. The tumours were frequently measured with a caliper in width (w), length (l) and height (h) and the tumour volume was calculated using the formula: (w/2×l/2×h/2×π×(4/3)). No treatments were administered during the study.

### Results and Conclusions

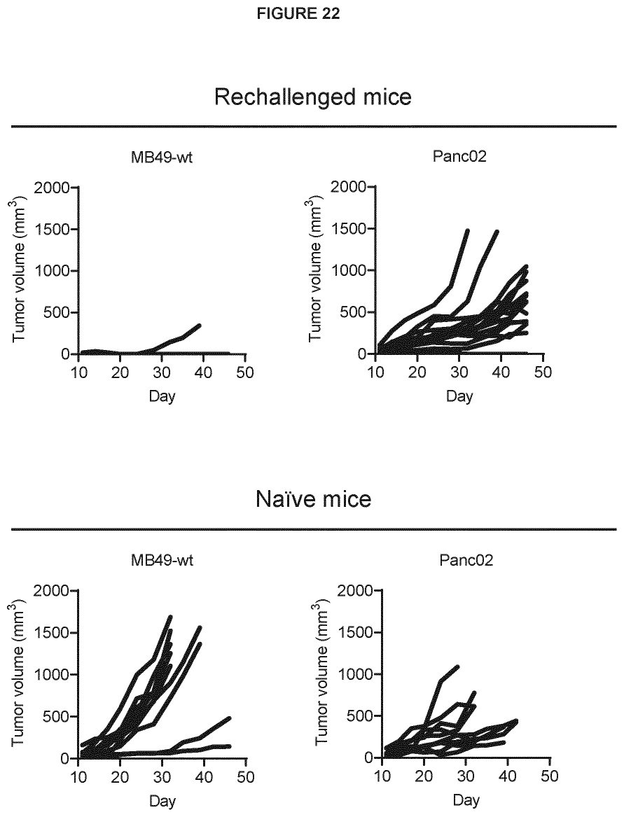

The data (shown in FIG. 22) demonstrate that in rechallenged mice, only the irrelevant Panc02 tumours are able to grow, while MB49-wt tumours do not display any detectable growth. In naïve mice, however, both MB49-wt and Panc02 tumours grow equally well. This suggests that the rechallenged mice have acquired immunological memory specifically to the MB49 tumour following treatment with 1132-3174.R, and that this immunological memory is not restricted to EpCAM.

### Example 21: Effect of the CD40-EpCAM Bispecific Antibody 1132-3174.R on Cross-Presentation of Necrotic Tumour Debris-Associated Antigen In Vitro

### Background and Aim

1132-3174.R is a CD40-EpCAM bispecific antibody intended to bind CD40 on dendritic cells (DC) and EpCAM on tumour debris or tumour extracellular vesicles such as exosomes, as EpCAM is overexpressed in a variety of tumours. These interactions would result in activation of DC as well as uptake of tumour debris, or tumour extracellular vesicles, by the DC. As tumour extracellular vesicles contain neoantigen, this would lead to improved cross-presentation of neoantigen-derived peptides, from DC to T cells, and subsequently result in a neoantigen-specific T cell expansion.

The aim of this study was to assess the effect of 1132-3174.R on DC in vitro cross-presentation of antigen from necrotic tumour debris and priming of CD8+ T cells using the model neoantigen ovalbumin (OVA).

### Materials and Methods

Human EpCAM and membrane-bound chicken OVA were transfected into the murine bladder carcinoma cell line MB49, generating a double transfected cell line, MB49-hEpCAM-OVA. MB49-hEpCAM-OVA cells and non-transfected MB49-wildtype (wt) cells were harvested and heat shocked at 45° C. for 10 min to induce cell death and incubated at 37° C. overnight.

OVA-specific T cells were obtained by collecting spleens from OT-1 mice (OVA T cell receptor transgenic, designed to recognize OVA peptide in the context of MHCI) and isolating CD8+ T cells using MACS according to the manufacturer's protocol (Miltenyi Biotec #130-104-075). The isolated CD8+OT-1 T cells were labeled with CellTrace Violet proliferative dye (CTV; Invitrogen C34557).

Spleens were collected from hCD40 transgenic mice and the tissue was digested with Liberase TL (Roche #05401020001) and DNase I (Roche #0104159001). CD11c+DC were isolated by MACS according to the manufacturer's protocol (Miltenyi Biotec #130-108-338).

In a 96-well plate, 100 000 DC/well were cultured with 200 000 CD8+ T cells/well and 100 000 necrotic MB49-hEpCAM-OVA or MB49-wt cells/well with or without 100 nM 1132-3174.R. After three days, cells were harvested, stained with fluorescently-labeled antibodies against murine CD45, MHC II (I-A/I-E) and CD8 followed by Fixable Viability Stain 780 (BD Biosciences). Samples were analyzed by flow cytometry to determine the frequency of CTV low (proliferating) CD8+ T cells.

### Results and Conclusions

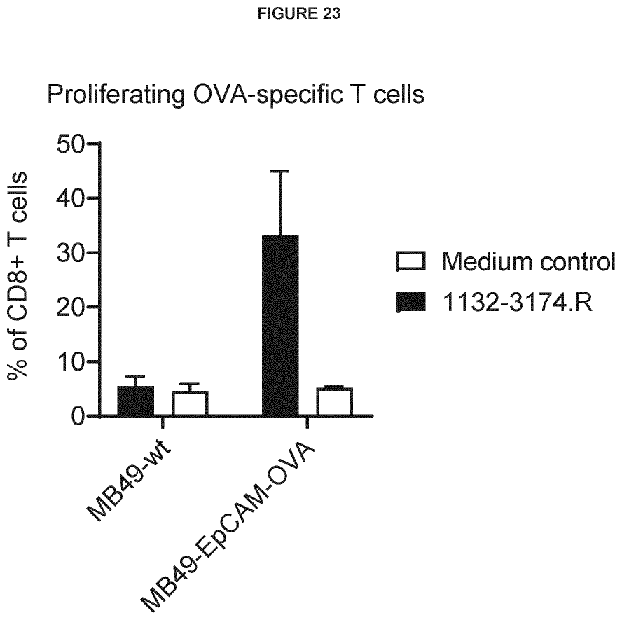

The data (shown in FIG. 23) demonstrate that 1132-3174.R induces increased proliferation of OVA-specific T cells compared to medium control in cultures with DC and necrotic MB49-hEpCAM-OVA, but not MB49-wt cells. This indicates that 1132-3174.R promotes uptake and cross-presentation of antigen present in necrotic cell debris.

### Example 22: Effect of the CD40-EpCAM Bispecific Antibody 1132-3174.R on Cross-Presentation of Necrotic Tumour Debris-Associated Antigen In Vivo

### Background and Aim

1132-3174.R is a CD40-EpCAM bispecific antibody intended to bind CD40 on dendritic cells (DC) and EpCAM on tumour debris or tumour extracellular vesicles such as exosomes, as EpCAM is overexpressed in a variety of tumours. These interactions would result in activation of DC as well as uptake of tumour debris, or tumour extracellular vesicles, by the DC. As tumour extracellular vesicles contain neoantigen, this would lead to improved cross-presentation of neoantigen-derived peptides, from DC to T cells, and subsequently result in a neoantigen-specific T cell expansion.

The aim of this study was to evaluate the effect of 1132-3174.R on T cell priming in vivo by use of ovalbumin (OVA) as a model neoantigen. Thus, hCD40tg mice that had received transfer of OT-1 T cells (OVA T cell receptor transgenic, designed to recognize OVA peptide in the context of MHCI) were immunized with heat-shocked MB49 tumour cells, double transfected with hEpCAM and OVA (MB49-hEpCAM-OVA), in order to assess the effect of 1132-3174.R on the priming of OT-1 T cells.

### Materials and Methods

Spleens were collected from OT-1 mice and CD8+ T cells isolated by MACS according to the manufacturer's protocol (Miltenyi Biotec #130-104-075). The isolated CD8+OT-1 T cells were labeled with CellTrace Violet proliferative dye (CTV; Invitrogen C34557) and 1×10⁶ cells transferred to female hCD40tg mice by i.v. injection into the tail vein.

The MB49-hEpCAM-OVA cell line was harvested and heat shocked at 45° C. for 10 min to induce cell death. The heat-shocked tumour cells were incubated at 37° C. overnight and 10×10⁶ cells injected i.p. to hCD40tg mice, 24 hours following the OT-1 T cell transfer. Additionally, the mice were administered 167 μg 1132-3174.R i.p. A group of vehicle-treated mice was also included.

Four days following immunization, the mice were sacrificed and spleens and inguinal lymph nodes collected. The organs were mashed through cell strainers to obtain single cell suspensions and the cells were subsequently Fc blocked and stained with an antibody cocktail containing fluorescently-labeled anti-mouse antibodies for CD11b, CD19, MHCII and NK1.1 (dump channel), and CD45, CD8, TCRVα2, TCRVβ5.1/Vβ5.2 as well as OVA (SIINFEKL) MHCI tetramer. The cells were also stained with Fixable Viability Stain 450 (BD Biosciences) to assess the cell viability. Samples were analysed by flow cytometry in order to determine the effect of 1132-3174.R on the frequency of viable CD45+CD8+ TCRVα2+ TCRVβ5.1/Vβ5.2+ proliferating (CTV low) OT-1 T cells.

### Results and Conclusions

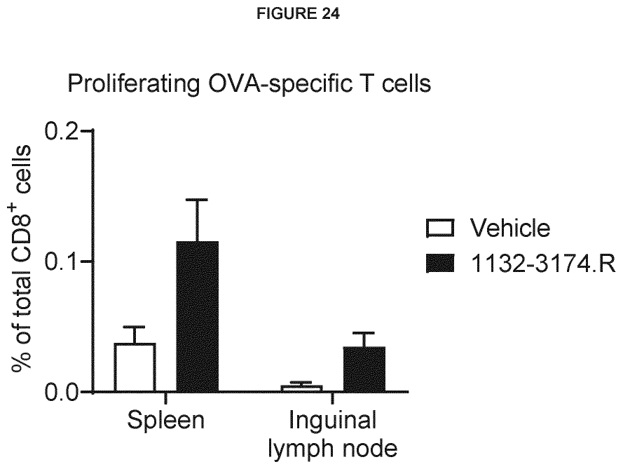

The data (shown in FIG. 24) demonstrate that 1132-3174.R treatment results in an increased frequency of the transferred, proliferating, OVA-specific CD8+ T cells in the spleen as well as the inguinal lymph nodes, compared to vehicle. This suggests that 1132-3174.R improves the priming of OVA-specific T cells in this model.

### Example 23: Effect of the CD40-EpCAM Bispecific Antibody 1132-3174.R on Cross-Presentation of Tumour Antigen In Vivo

### Background and Aim

1132-3174.R is a CD40-EpCAM bispecific antibody intended to bind CD40 on dendritic cells (DC) and EpCAM on tumour debris or tumour extracellular vesicles such as exosomes, as EpCAM is overexpressed in a variety of tumours. These interactions would result in activation of DC as well as uptake of tumour debris, or tumour extracellular vesicles, by the DC. As tumour extracellular vesicles contain neoantigen, this would lead to improved cross-presentation of neoantigen-derived peptides, from DC to T cells, and subsequently result in a neoantigen-specific T cell expansion.

The aim of this study was to evaluate the effect of 1132-3174.R on T cell priming in vivo, in tumour-bearing mice, by use of ovalbumin (OVA) as a model neoantigen. Thus, hCD40tg mice were inoculated s.c. with MB49 tumour cells, double transfected with hEpCAM and OVA (MB49-hEpCAM-OVA). These mice also received adoptive transfer of OT-1 T cells (OVA T cell receptor transgenic, designed to recognize OVA peptide in the context of MHCI), and were treated with 1132-3174.R to assess its effect on the priming of the transferred OT-1 T cells. To be able to study all OT-1 cells that are primed in the tumour-draining lymph nodes, the mice were also treated with the drug FTY720, to prevent T cell egress from lymph nodes.

### Materials and Methods

Female hCD40tg mice of 10-13 weeks of age were inoculated with 5×10⁵ MB49-hEpCAM-OVA cells s.c. in the right flank.

On day 17 post-inoculation, spleens were collected from a cohort of OT-1 mice and CD8+ T cells isolated by MACS according to the manufacturer's protocol (Miltenyi Biotec #130-104-075). The isolated CD8+OT-1 T cells were labeled with CellTrace Violet proliferative dye (CTV; Invitrogen C34557) and 1×10⁶ cells transferred to the MB49-hEpCAM-OVA tumour-bearing mice by i.v. injection into the tail vein.

Twenty-four hours following the OT-1 T cell transfer, the mice were administered 417 μg 1132-3174.R i.p. A group of vehicle-treated mice was also included. An additional 24 hours following the therapy treatment, the mice were also administered 20 μg FTY720 (Cayman Chemicals #10006292) in order to prevent egress of any OT-1 T cells that have been primed in the tumour-draining lymph nodes.

Two days following FTY720 treatment, on day 21 post-inoculation, the mice were sacrificed and tumour-draining (inguinal) lymph nodes collected. The lymph nodes were mashed through cell strainers to obtain single cell suspensions and the cells were subsequently Fc blocked and stained with an antibody cocktail containing fluorescently-labeled anti-mouse antibodies for CD11b, CD19, MHCII, NK1.1 and Ter119 (dump channel), and CD3, CD4 and CD8, as well as OVA (SIINFEKL) MHCI tetramer. The cells were also stained with Fixable Viability Stain 780 (BD Biosciences) to assess the cell viability. Samples were analysed by flow cytometry in order to determine the effect of 1132-3174.R on the frequency of viable CD3+ CD8+ OVA-MHCI tetramer+ T cells.

### Results and Conclusions

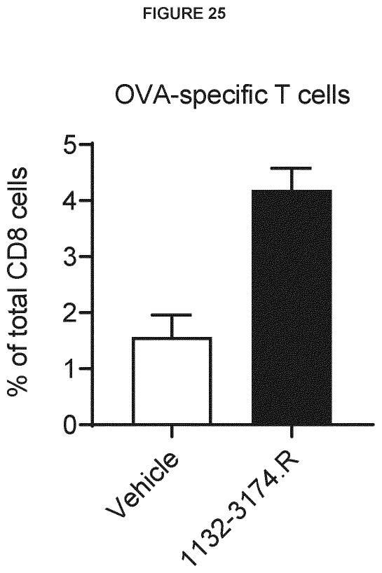

The data (shown in FIG. 25) demonstrate that 1132-3174.R treatment results in an increased frequency of the OVA-specific CD8+ T cells in the tumour-draining lymph nodes, compared to vehicle. This suggests that 1132-3174.R improves the priming of OVA-specific T cells in mice bearing tumours which express OVA.

### Example 24: Effect of the CD40-5T4 Bispecific Antibody 1132-1210.M on Co-Localization (Such as, Internalization) of 5T4+ Tumour Cell Debris in a CD40-Expressing Cell Line

### Background and Aim

1132-1210.M is a CD40-5T4 bispecific antibody in the Morrison format wherein 1132 refers to its CD40 agonist domain and 1210 to its 5T4-binding, tumour-targeting, domain. The antibody has been LALA-mutated to silence Fcγ receptor binding.

The aim of this study was to assess the effect of 1132-1210.M on co-localization (such as, internalization) of cell debris from 5T4+ tumour cell lines into a CD40-expressing cell line. The cell debris were obtained from the murine CT26 cell line transfected with human 5T4.

### Materials and Methods

Human 5T4 was transfected into the murine CT26 colon carcinoma cell line to generate clones with varying expression (low, intermediate and high) of human 5T4. The cell surface density of human 5T4 was measured on the various CT26 clones by use of the Quantum Simply Cellular kit (Bangs Laboratories) according to the manufacturer's protocol.

CT26-wt and the three CT26-h5T4 cell clones were stained with the fluorescent membrane dye PKH26 (Sigma-Aldrich) followed by heat shock at 45° C. for 10 min to induce cell death. Heat-shocked tumour cells were incubated at 37° C. overnight, spun down and supernatant containing tumour cell debris was collected.

CD40+ Raji cells were labeled with nuclear stain Hoechst 33342 (Thermo Fisher) at a concentration of 0.045 μg/ml and cultured with CT26-wt or CT26-h5T4 tumour cell debris. Titrated concentrations of the bispecific antibody 1132-1210.M or the monoclonal CD40 antibody 1132.m2 were added to the cultures of Raji cells and tumour cell debris. Cells were imaged every second hour using the live cell imaging system Cytation5 (BioTek). Images were analyzed and the number of tumour cell debris localized in Raji cells was quantified using Gen5 software (BioTek).

### Results and Conclusions

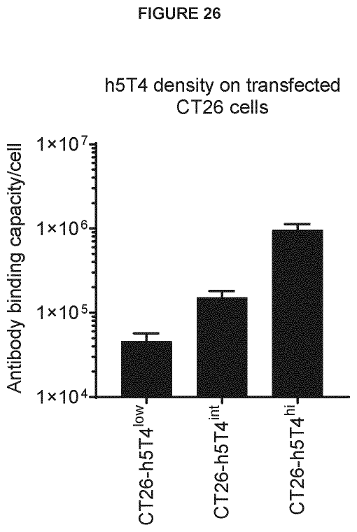

5T4 quantification was performed on the transfected CT26 cells to determine the density of human 5T4. The quantification data (as shown in FIG. 26) demonstrate that CT26-h5T4^{low} display a 5T4 density of approx. 0.05×10⁶ molecules per cell, CT26-h5T4^{int} display a 5T4 density of approx. 0.15×10⁶ molecules per cell and CT26-h5T4^{hi} display a 5T4 density of approx. 1×10⁶ molecules per cell.

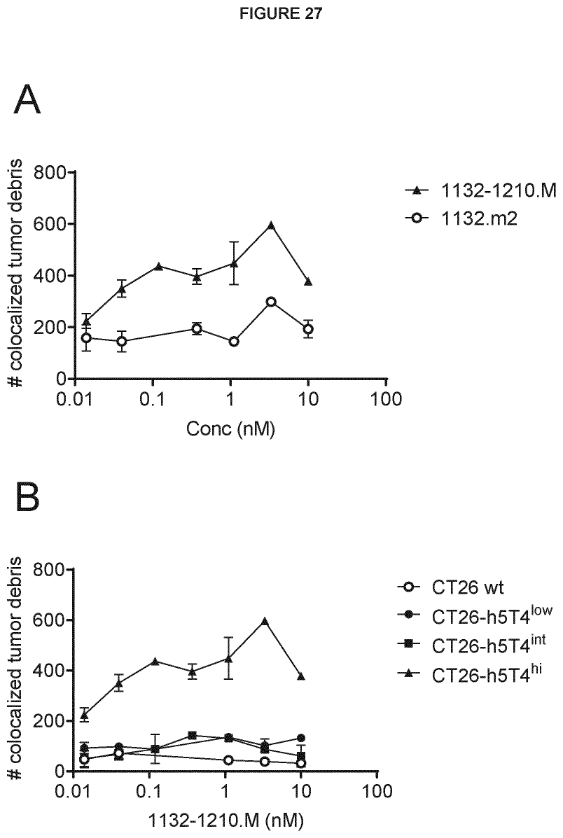

The internalization data (as shown in FIG. 27) demonstrate that 1132-1210.M mediates increased localization of 5T4+ tumour cell debris in CD40+ cells, whereas the CD40 monoclonal antibody 1132.m2 does not (A). Further, the data also demonstrate that this effect is achieved only when tumour debris from CT26-h5T4^{hi} cells are used (B). Thus, a 5T4 density of at least between 0.15×10⁶-1×10⁶ molecules per tumour cell is required for 1132-1210.M to mediate efficient internalization of tumour cell debris.

### Example 25: Effect of the CD40-EpCAM Bispecific Antibody 1132-3174.R on Internalization of EpCAM+ Tumour Cell Debris in a CD40-Expressing Cell Line

### Background and Aim

1132-3174.R is a CD40-EpCAM bispecific antibody in the RUBY™ format wherein 1132 refers to the CD40-binding domain and 3174 to the EpCAM-binding, tumour-targeting, domain. The antibody has been LALA-mutated to silence Fcγ receptor binding.

The aim of this study was to assess the effect of 1132-3174.R on internalization of cell debris from EpCAM+ tumour cell lines into a CD40-expressing cell line. The cell debris were obtained from different human tumour cell lines with varying endogenous expression of EpCAM.

### Materials and Methods

The cell surface density of EpCAM was measured on the tumour cell lines BxPC3, MCF7, JAR and JEG by use of the Quantum Simply Cellular kit (Bangs Laboratories) according to the manufacturer's protocol.

The tumour cell lines BxPC3, MCF7, JAR and JEG were stained with the fluorescent membrane dye PKH26 (Sigma-Aldrich) followed by heat shock at 45° C. for 10 min to induce cell death. Heat-shocked tumour cells were incubated at 37° C. overnight, spun down and supernatant containing tumour cell debris was collected.

CD40+ Raji cells were labeled with nuclear stain Hoechst 33342 (Thermo Fisher) at a concentration of 0.045 μg/ml and cultured with BxPC3, MCF7, JAR or JEG tumour cell debris. Titrated concentrations of the bispecific antibody 1132-3174.R or the monoclonal CD40 antibody 1132.m2 were added to the cultures of Raji cells and tumour cell debris. Cells were imaged every second hour using the live cell imaging system Cytation5 (BioTek). Images were analyzed and the number of tumour cell debris localized in Raji cells was quantified using Gen5 software (BioTek).

### Results and Conclusions

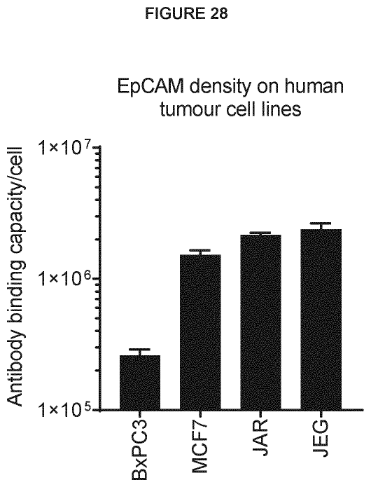

EpCAM quantification was performed on the tumour cell lines BxPC3, MCF7, JAR and JEG to determine the density of EpCAM. The quantification data (as shown in FIG. 28) demonstrate that BxPC3 display an EpCAM density of approx. 2.5×10⁵ molecules per cell (EpCAM^{low}, MCF7 display an EpCAM density of approx. 1.5×10⁶ molecules per cell (EpCAM^{int}), JAR display an EpCAM density of approx. 2×10⁶ molecules per cell (EpCAM^{hi}) and JEG display an EpCAM density of approx. 2.5×10⁶ molecules per cell (EpCAM^{hi}).

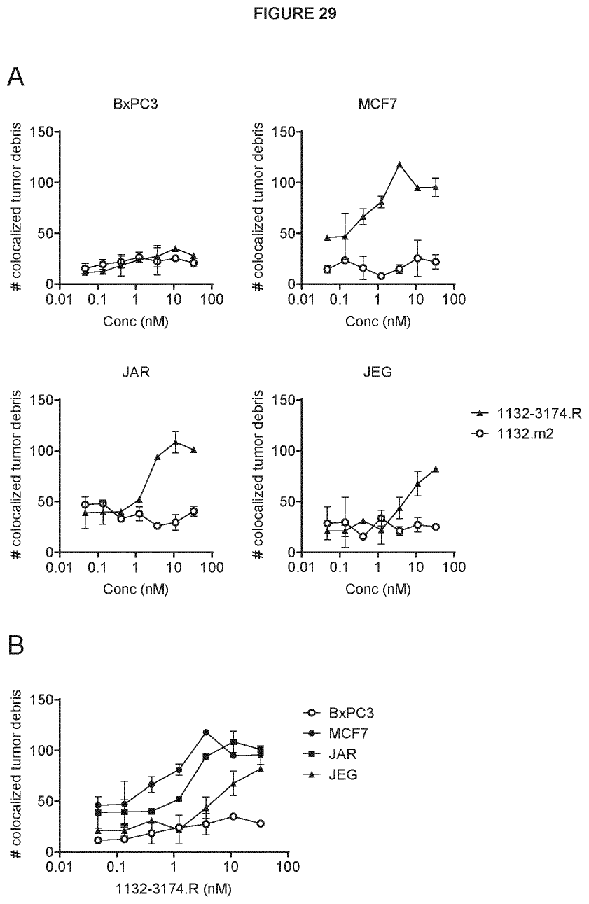

The internalization data (as shown in FIG. 29) demonstrate that 1132-3174.R mediates increased localization of EpCAM+ tumour cell debris in CD40+ cells, whereas the CD40 monoclonal antibody 1132.m2 does not (A). Further, the data also demonstrate that this effect is achieved only when tumour debris from EpCAM^{int} (MCF7) or EpCAM^{hi} cells (JAR or JEG) are used (A and B). Thus, an EpCAM density of at least between 2.5×10⁵-1.5×10⁶ molecules per tumour cell is required for 1132-3174.R to mediate efficient internalization of tumour cell debris.

### Example 26: Effect of the CD40-HER2 Bispecific Antibody 1132-Trastuzumab® on Internalization of HER2+ Tumour Cell Debris in a CD40-Expressing Cell Line

### Background and Aim

1132-Trastuzumab® is a CD40-HER2 bispecific antibody in the RUBY™ format wherein 1132 refers to the CD40-binding domain and Trastuzumab to the HER2-binding, tumour-targeting, domain. The antibody has been LALA-mutated to silence Fcγ receptor binding.

The aim of this study was to assess the effect of 1132-Trastuzumab® on internalization of cell debris from HER2+ tumour cell lines into a CD40-expressing cell line. The cell debris were obtained from different human tumour cell lines with varying endogenous expression of HER2.

### Materials and Methods

The cell surface density of HER2 was measured on the tumour cell lines BxPC3, HT29, MCF7, LS174T and SK-OV-3 by use of the Quantum Simply Cellular kit (Bang Laboratories) according to the manufacturer's protocol.

The tumour cell lines BxPC3, HT29, MCF7, LS174T and SK-OV-3 were stained with the fluorescent membrane dye PKH26 (Sigma-Aldrich) followed by heat shock at 45° C. for 10 min to induce cell death. LS174T cells where HER2 had been knocked-down (LS174T-HER2 KO) were also included as a negative control. Heat-shocked tumour cells were incubated at 37° C. overnight, spun down and supernatant containing tumour cell debris was collected.

CD40+ Raji cells were labeled with nuclear stain Hoechst 33342 (Thermo Fisher) at a concentration of 0.045 μg/ml and cultured with BxPC3, HT29, MCF7, LS174T, LS174T-HER2 KO and SK-OV-3 tumour cell debris. Titrated concentrations of the bispecific antibody 1132-Trastuzumab® or the monoclonal CD40 antibody 1132.m2 were added to the cultures of Raji cells and tumour cell debris. Cells were imaged every second hour using the live cell imaging system Cytation5 (BioTek). Images were analyzed and the number of tumour debris localized in Raji cells was quantified using Gen5 software (BioTek).

### Results and Conclusions

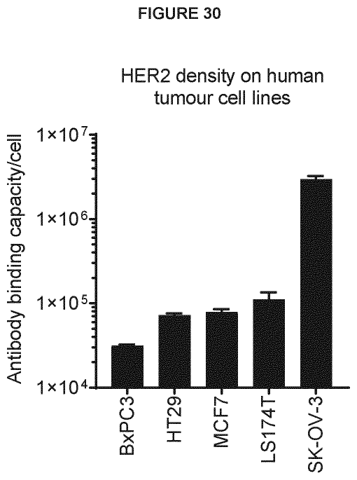

HER2 quantification was performed on the tumour cell lines BxPC3, HT29, MCF7, LS174T and SK-OV-3 to determine the density of HER2. The quantification data (as shown in FIG. 30) demonstrate that BxPC3 display a HER2 density of approx. 3×10⁴ molecules per cell (HER2^{low}), HT29 and MCF7 display a HER2 density of approx. 7.5×10⁴ molecules per cell (HER2^{int}), LS174T display a HER2 density of approx. 1×10⁵ molecules per cell (HER2^{int}) and SK-OV-3 display a HER2 density of approx. 3×10⁶ molecules per cell (HER2^{hi}). The LS174T-HER2 KO display no detectable HER2 molecules on the cell surface (data not shown).

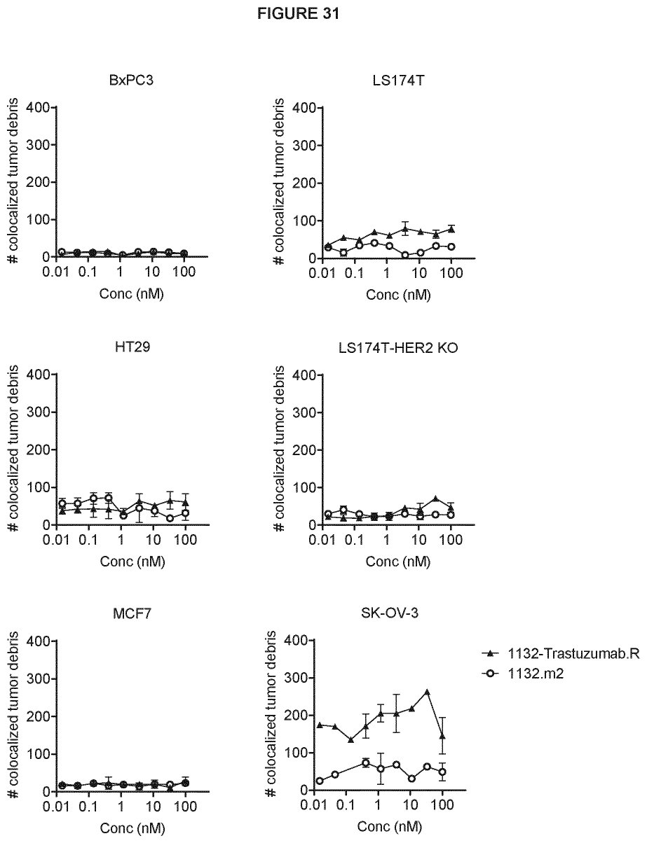

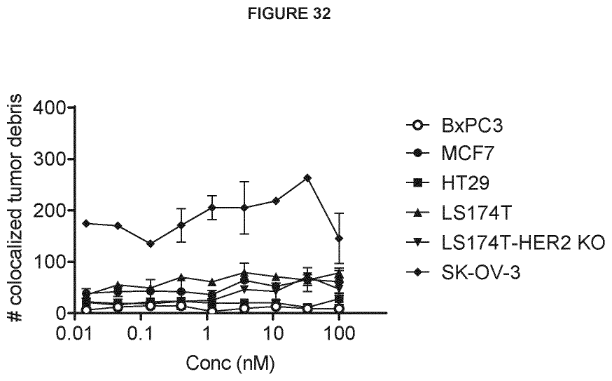

The internalization data (as shown in FIGS. 31 and 32) demonstrate that 1132-Trastuzumab® mediates increased localization of HER2+ tumour cell debris in CD40+ cells, whereas the CD40 monoclonal antibody 1132.m2 does not. Further, the data also demonstrate that this effect is achieved only when tumour debris from HER2^{hi} cells (SK-OV-3) are used (FIGS. 31 and 32). Thus, a HER2 density of at least between 1×10⁵-3×10⁶ molecules per tumour cell is required for 1132-Trastuzumab® to mediate efficient internalization of tumour cell debris.

### Example 27: Effect of the DEC-205-EpCAM Bispecific Antibody 3G9-3174.R on Internalization of EpCAM+ Tumour Cell Debris in a DEC-205-Expressing Cell Line

### Background and Aim

3G9-3174.R is a DEC-205-EpCAM bispecific antibody in the RUBY™ format wherein 3G9 refers to the DEC-205-binding domain and 3174 to the EpCAM-binding, tumour-targeting, domain. The antibody has been LALA-mutated to silence Fcγ receptor binding.

The aim of this study was to assess the effect of 3G9-3174.R on internalization of cell debris from EpCAM+ tumour cell lines into a DEC-205-expressing cell line. The cell debris were obtained from different human tumour cell lines with varying endogenous expression of EpCAM.

### Materials and Methods

The tumour cell lines BxPC3, MCF7 and JAR were stained with the fluorescent membrane dye PKH26 (Sigma-Aldrich) followed by heat shock at 45° C. for 10 min to induce cell death. Heat-shocked tumour cells were incubated at 37° C. overnight, spun down and supernatant containing tumour cell debris was collected.

DEC-205+ Raji cells were labeled with nuclear stain Hoechst 33342 (Thermo Fisher) at a concentration of 0.045 μg/ml and cultured with BxPC3, MCF7 or JAR tumour cell debris. The bispecific antibody 3G9-3174.R, or 1188-3174.R, an isotype-EpCAM bispecific antibody, were added to the cultures of Raji cells and tumour cell debris at a concentration of 1.2 nM. Cells were imaged every second hour using the live cell imaging system Cytation5 (BioTek). Images were analyzed and the number of tumour cell debris localized in Raji cells was quantified using Gen5 software (BioTek).

### Results and Conclusions

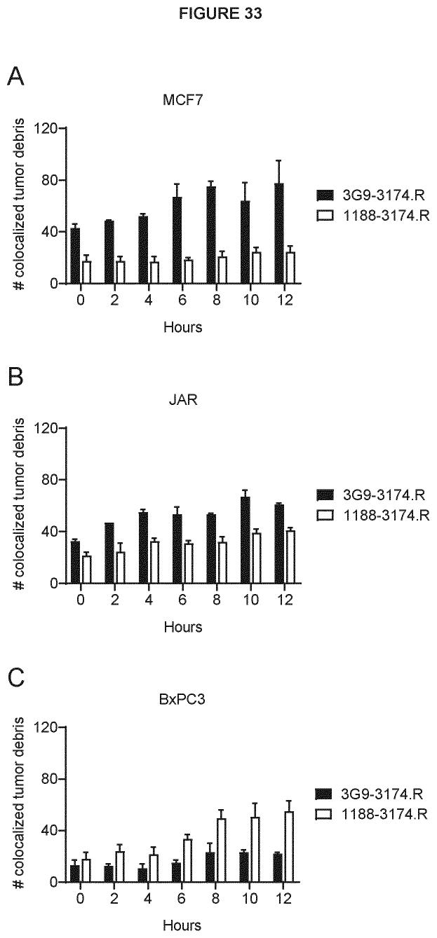

The internalization data (as shown in FIG. 33) demonstrate that 3G9-3174.R mediates increased localization of the EpCAM^{int} debris from MCF7 and EpCAM^{hi} debris from JAR by DEC-205+ cells, whereas the isotype-EpCAM bispecific antibody 1188-3174.R does not (A and B). Further, the data also demonstrate that this effect is not achieved when debris from the EpCAM^{low} cell line BxPC3 are used (C). Thus, an EpCAM density of at least between 2.5×10⁵-1.5×10⁶ molecules per tumour cell is required for 3G9-3174.R to mediate efficient internalization of tumour cell debris.

### Example 28: Effect of the CD40-EpCAM Bispecific Antibody 1132-3174.R on Cross-Presentation of Exosome-Associated Antigen In Vitro

### Background and Aim

1132-3174.R is a CD40-EpCAM bispecific antibody intended to bind CD40 on dendritic cells (DC) and EpCAM on tumour debris or tumour extracellular vesicles such as exosomes (30-200 nm diameter), as EpCAM is overexpressed in a variety of tumours. These interactions would result in activation of DC as well as uptake of tumour exosomes, or tumour extracellular vesicles, by the DC. As tumour extracellular vesicles contain neoantigen, this would lead to improved cross-presentation of neoantigen-derived peptides, from DC to T cells, and subsequently result in a neoantigen-specific T cell expansion.

The aim of this study was to assess the effect of 1132-3174.R on DC in vitro cross-presentation of antigen from tumour-derived exosomes and priming of CD8+ T cells using the model neoantigen ovalbumin (OVA), and compare it to the bispecific isotype-EpCAM antibody 1188-3174.R.

### Materials and Methods

Human EpCAM and membrane-bound chicken OVA were transfected into the murine bladder carcinoma cell line MB49, generating a double transfected cell line, MB49-hEpCAM-OVA.

Exosomes were isolated from cell culture media from MB49-hEpCAM-OVA cells cultured under serum-free conditions for 24 hours by incubating with Total Exosome Isolation Reagent (Invitrogen #4478359) at 4° C. overnight. After incubation, the sample was centrifuged at 10,000×g for one hour at 4° C. Supernatant was discarded and exosomes collected by resuspending the pellet at the bottom of the tube. Exosomes were filtered using 0.22 μm Millex-GV (Merck Millipore #SLGV033RS) in order to remove larger particles. Exosomes were then filtered using Amicon Ultra-4 Centrifugal Filter Devices (Merck Millipore #UFC810024) with 100,000 molecular weight cut-off in order to remove smaller particles. Purified exosomes were analyzed by Dynamic Light Scattering (DLS) using Uncle (Unchained Labs) to ensure that both larger and smaller particles have been removed. To assess the yield of isolated exosomes, quantification of total protein was measured using Pierce BCA Protein Assay Kit (Thermo Scientific #23227).

OVA-specific T cells were obtained by collecting spleens from OT-1 mice (OVA T cell receptor transgenic, designed to recognize OVA peptide in the context of MHCI) and isolating CD8+ T cells using MACS according to the manufacturer's protocol (Miltenyi Biotec #130-104-075). The isolated CD8+OT-1 T cells were labeled with CellTrace Violet proliferative dye (CTV; Invitrogen C34557).

Spleens were collected from hCD40 transgenic mice and the tissue was digested with Liberase TL (Roche #05401020001) and DNase I (Roche #0104159001). CD11c+DC were isolated by MACS according to the manufacturer's protocol (Miltenyi Biotec #130-108-338).

In a 96-well plate, 100 000 DC/well were cultured with 200 000 CD8+ T cells/well and a 2-fold serial dilution of exosomes from MB49-hEpCAM-OVA cells/well with 100 nM 1132-3174.R or 1188-3174.R. After four days, cells were harvested, stained with fluorescently-labeled antibodies against murine CD45, MHC II (I-A/I-E) CD11c and CD8 followed by Fixable Viability Stain 780 (BD Biosciences #565388). Samples were analyzed by flow cytometry to determine the frequency of CTV low (proliferating) CD8+ T cells.

### Results and Conclusions

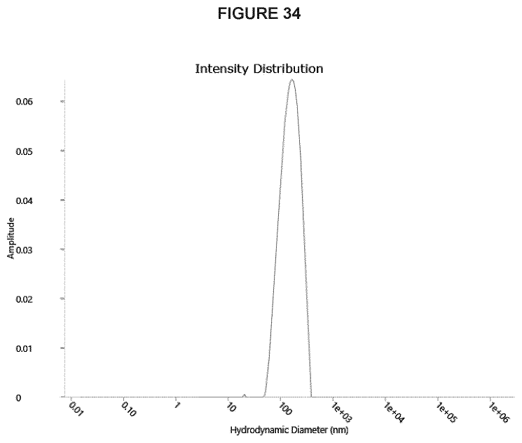

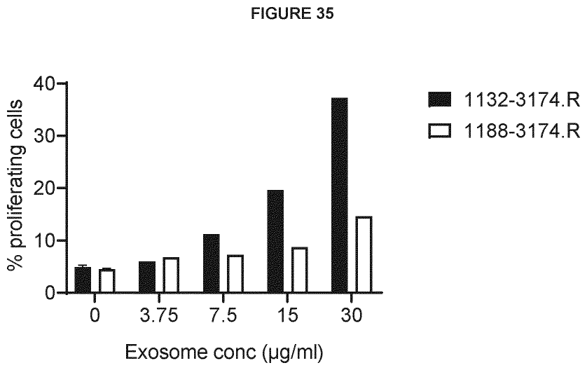

Dynamic Light Scattering (shown in FIG. 34) demonstrates that the isolated exosomes were a homogenous population without contamination of larger whole cells. Further, the data (shown in FIG. 35) also demonstrate that 1132-3174.R induces increased proliferation of OVA-specific T cells compared to 1188-3174.R in cultures with DC and MB49-hEpCAM-OVA-derived exosomes. This indicates that 1132-3174.R promotes uptake and cross-presentation of antigen present in exosomes.

### Example 29: Anti-Tumour Effect of the CD40-EpCAM Bispecific Antibody 1132-3174.R

### Background and Aim

1132-3174.R is a CD40-EpCAM bispecific antibody in RUBY™ format wherein 1132 refers to its CD40 agonist domain and 3174 to its EpCAM-binding, tumour-targeting domain. The antibody has been LALA-mutated to silence Fcγ receptor binding.

The aim of this study was to evaluate the anti-tumour effect of 1132-3174.R in human CD40 transgenic (hCD40tg) mice inoculated with murine MB49 tumours transfected with human EpCAM (MB49-hEpCAM) or MB49-wt (hEpCAM negative) tumours.

### Materials and Methods

Female hCD40tg mice of 13-16 weeks of age were inoculated with either 2.5×10⁵ MB49-wt or MB49-hEpCAM cells s.c. in the right flank. On days 10, 13 and 16 after inoculation, the mice were administered i.p. with 100 μg of wildtype CD40 monospecific antibody 1132 or 100 μg of the LALA-mutated equivalent 1132.m2. Alternatively, the mice received 167 μg 1132-3174.R (dose of molecular mass equivalence to the monospecific antibodies) or 417 μg 1132-3174.R (dose 2.5 fold higher in terms of molecular mass, compared to monospecific antibodies). A group of vehicle-treated mice was also included. The mice were kept in the study until the individual tumour volume reached the ethical limit of 2000 mm³, at which point the mice were sacrificed.

### Results and Conclusions

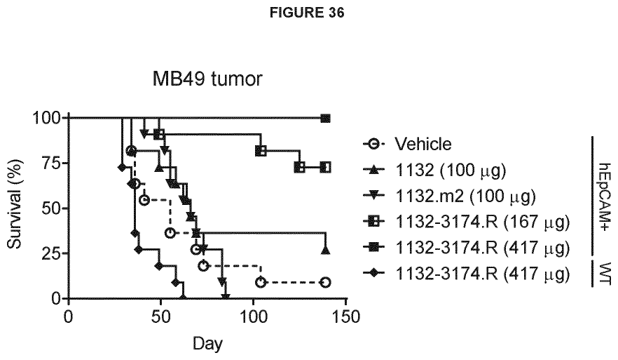

The data (shown in FIG. 36) demonstrate that treatment with 1132-3174.R significantly improves the survival compared to vehicle-treated mice, as well as mice treated with a molecular mass equivalent dose of 1132. A 2.5-fold higher dose of 1132-3174.R results in complete tumour eradication, and 100% survival of the mice. Additionally, in mice bearing MB49-wt tumours administered the same high dose of 1132-3174.R, the anti-tumour effect of 1132-3174.R is completely diminished. Thus, 1132-3174.R has a potent, EpCAM-dependent anti-tumour effect in the MB49 tumour model.

1. A bispecific polypeptide comprising a first binding domain, designated B1, which is capable of binding specifically to CD40, and a second binding domain, designated B2, which is capable of specifically binding to a tumour cell-associated antigen (TAA).

2. A bispecific polypeptide according to claim 1, wherein the first and/or second binding domains are/is selected from the group consisting of antibodies and antigen-binding fragments thereof.

3. A bispecific polypeptide according to claim 2 wherein the antigen-binding fragment is selected from the group consisting of: Fv fragments (such as a single chain Fv fragment, or a disulphide-bonded Fv fragment), Fab-like fragments (such as a Fab fragment; a Fab′ fragment or a F(ab)₂ fragment) and domain antibodies.

4. A bispecific polypeptide according to any one of the preceding claims wherein the polypeptide is a bispecific antibody.

5. A bispecific polypeptide according to claim 4 wherein:
(a) binding domain B1 and/or binding domain B2 is an intact IgG antibody;
(b) binding domain B1 and/or binding domain B2 is an Fv fragment;
(c) binding domain B1 and/or binding domain B2 is a Fab fragment; and/or
(d) binding domain B1 and/or binding domain B2 is a single domain antibody.

6. A bispecific polypeptide according to claim 4 or 5 wherein the bispecific antibody comprises a human Fc region or a variant of a said region, where the region is an IgG1, IgG2, IgG3 or IgG4 region, preferably an IgG1 or IgG4 region.

7. A bispecific polypeptide according to claim 6 wherein the Fc exhibits no or very low affinity for FcγR.

8. A bispecific polypeptide according to claim 6 or 7 wherein the Fc region is a variant of a human IgG1 Fc region comprising a mutation at one or more of the following positions:
L234, L235, P239, D265, N297 and/or P329.

9. A bispecific polypeptide according to claim 8 wherein alanine is present at the mutated position(s).

10. A bispecific polypeptide according to claim 9 wherein the Fc region is a variant of a human IgG1 Fc region comprising the double mutations L234A and L235A.

11. A bispecific polypeptide according to any one of claims 4 to 10 wherein the bispecific antibody is selected from the groups consisting of:
(a) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
(b) monovalent bispecific antibodies, such as a DuoBody® or a ‘knob-in-hole’ bispecific antibody (for example, an scFv-KIH, scFv-KIH^{r}, a BiTE-KIH or a BiTE-KIH^{r});
(c) scFv₂-Fc bispecific antibodies (for example, ADAPTIR™ bispecific antibodies);
(d) BiTE/scFv₂ bispecific antibodies;
(e) DVD-Ig bispecific antibodies;
(f) DART-based bispecific antibodies (for example, DART₂-Fc or DART);
(g) DNL-Fab₃ bispecific antibodies; and
(h) scFv-HSA-scFv bispecific antibodies.

12. A bispecific polypeptide according to claim 11 wherein the bispecific antibody is an IgG-scFv bispecific antibody.

13. A bispecific polypeptide according to any one of the preceding claims wherein binding domain B1 and binding domain B2 are fused directly to each other.

14. A bispecific polypeptide according to any one of claims 1 to 12 wherein binding domain B1 and binding domain B2 are joined via a polypeptide linker.

15. A bispecific polypeptide according to claim 14 wherein the linker is selected from the group consisting of the amino acid sequence SGGGGSGGGGS (SEQ ID NO: 162), SGGGGSGGGGSAP (SEQ ID NO: 163), NFSQP (SEQ ID NO: 164), KRTVA (SEQ ID NO: 165), GGGSGGGG (SEQ ID NO: 166), GGGGSGGGGS (SEQ ID NO: 167), GGGGSGGGGSGGGGS (SEQ ID NO: 168), GSTSGSGKPGSGEGSTKG (SEQ ID NO: 169), THTCPPCPEPKSSDK (SEQ ID NO: 170), GGGS (SEQ ID NO: 171), EAAKEAAKGGGGS (SEQ ID NO: 172), EAAKEAAK (SEQ ID NO: 173), or (SG)m, where m=1 to 7.

16. A bispecific polypeptide according to any one of the preceding claims wherein one of B1 or B2 is an immunoglobulin molecule, and one of B1 or B2 is a Fab fragment, wherein the Fab fragment is fused to the C-terminus of the heavy chain of the immunoglobulin via the light chain of the Fab fragment.

17. A bispecific polypeptide according to claim 16 wherein the bispecific polypeptide comprises one or more mutations to promote association of the heavy chain polypeptide of the immunoglobulin with the light chain polypeptide of the immunoglobulin and/or to promote association of the heavy chain polypeptide of the Fab with the light chain polypeptide of the Fab.

18. A bispecific polypeptide according to claim 17 wherein the one or more mutations prevent the formation of aggregates and a Fab by-product.

19. A bispecific polypeptide according to claim 18, wherein the mutations prevent formation of aggregates and Fab by-products by generating steric hindrance and/or incompatibility between charges.

20. A bispecific polypeptide according to any one of claims 17 to 19 wherein the polypeptide comprises one or more mutation pairs each comprising two functionally compatible mutations.

21. A bispecific polypeptide according to any one of the preceding claims, wherein the polypeptide is incapable of inducing antibody-dependent cell cytotoxicity (ADCC), antibody-dependent cellular phagocytosis (ADCP) and/or complement-dependent cytotoxicity (CDC).

22. A bispecific polypeptide according to any one of the preceding claims, wherein the polypeptide is capable of inducing tumour immunity.

23. A bispecific polypeptide according to any one of the preceding claims, wherein the polypeptide is capable of inducing:
(a) tumour-specific immune activation; and/or
(b) activation of dendritic cells; and/or
(c) internalisation of associated tumour debris and/or extracellular vesicles containing tumour cell-associated antigens as well as tumour neoantigens; and/or
(d) cross-presentation of peptides derived from internalised tumour antigens on MHC; and/or
(e) priming and activation of effector T cells; and/or
(f) direct tumoricidal effects, selected from the list consisting of: apoptosis, antibody-dependent cellular cytotoxicity (ADCC) and complement-dependent cytotoxicity (CDC).

24. A bispecific polypeptide according to any one of the preceding claims wherein binding domain B1 binds to human CD40 with a K_{D} of less than 100×10⁻⁹M or less than 50×10⁻⁹M or less than 25×10⁻⁹M, preferably less than 10, 9, 8, 7, or 6×10⁻⁹M, more preferably less than 5, 4, 3, 2, or 1×10⁻⁹M, most preferably less than 9×10⁻¹⁹M.

25. A bispecific polypeptide according to any one of the preceding claims, wherein B1 exhibits at least one of the following functional characteristics when present independently of B2:
(a) binding to human CD40 with a K_{D} value which is less than 100×10⁻⁹M, more preferably less than 10×10⁻⁹M;
(b) does not bind to murine CD40; and
(c) does not bind to other human TN FR superfamily members, for example human CD137 or OX40

26. A bispecific polypeptide according to any one of the preceding claims, wherein binding domain B1 comprises one or more heavy chain CDR sequences selected from those in Table C(1) and/or wherein binding domain B1 comprises one or more light chain CDR sequences selected from those in Table C(2).

27. A bispecific polypeptide according to any one of the preceding claims, wherein binding domain B1 comprises one, two or three light chain CDR sequences from a particular row for an individual antibody reference in Table C(2), and/or one, two or three heavy chain CDR sequences from the corresponding row for the antibody with the same reference in Table C(1).

28. A bispecific polypeptide according to any one of the preceding claims wherein binding domain B1 comprises all three heavy chain CDR sequences of a particular antibody reference as shown in Table C(1), and/or all three light chain CDR sequences of an antibody reference as shown in Table C(2), or wherein binding domain B1 comprises a heavy chain VH sequence and/or a light chain VL sequence as shown in Table A.

29. A bispecific polypeptide according to any one of the preceding claims, wherein B1 comprises any one, two, three, four, five or all six features independently selected from the following:
(a) a heavy chain CDR1 sequence which consists of the sequence “G, F, T, F, S, S, Y, A”;
(b) a heavy chain CDR2 sequence which is 8 amino acids in length and comprises the consensus sequence: “I, G/S, S/G, Y/S, G/S, G/S, G/Y/S, T”;
(c) a heavy chain CDR3 sequence which is 9 to 12 amino acids in length and which comprises the consensus sequence of: “A, R, Y/R/G, Y/P/V/-, N/S/V, F/Y/W, G/H/S, -/S, -/V, M/F, D, Y”
(d) a light chain CDR1 sequence which consists of the sequence: “Q, S, I, S, S, Y”;
(e) a light chain CDR2 sequence which consists of the sequence: “A, A, S”;
(f) a light chain CDR3 sequence which is 9 amino acids in length and comprises the consensus sequence: “Q, Q, Y/S, G/Y, R/S/V, N/A/Y/T, P, P/F/Y, T”.

30. A bispecific polypeptide according to any one of the preceding claims wherein binding domain B1 comprises:
(a) the three CDRs of the heavy chain and/or the three CDRs of the light chain of antibody 1132/1133 (SEQ ID NOs: 73, 74 and 75; and/or 90, 91 and 92); or
(b) the three CDRs of the heavy chain and/or the three CDRs of the light chain of antibody 1107/1108 (SEQ ID NOs: 73, 78 and 80; and/or SEQ ID NOs: 90, 91 and 95); or
(c) the three CDRs of the heavy chain and/or the three CDRs of the light chain of antibody 1150/1151 (SEQ ID NOs: 73, 76 and 77; and/or SEQ ID NOs: 90, 91 and 93); or
(d) the three CDRs of the heavy chain and/or the three CDRs of the light chain of antibody 1140/1135 (SEQ ID NOs: 73, 78 and 79; and/or SEQ ID NOs: 90, 91 and 94); or
(e) the three CDRs of the heavy chain and/or the three CDRs of the light chain of antibody ADC-1013 (SEQ ID NOs: 81, 82 and 83; and/or SEQ ID NOs: 96, 97, and 98); or
(f) the three CDRs of the heavy chain and/or the three CDRs of the light chain of antibody APX005 (SEQ ID NOs: 84, 85 and 86; and/or SEQ ID NOs: 99, 100, and 101); or
(g) the three CDRs of the heavy chain and/or the three CDRs of the light chain of antibody 21.4.1 (SEQ ID NOs: 87, 88 and 89; and/or SEQ ID NOs: 102, 103, and 104).

31. A bispecific polypeptide according to any one of the preceding claims wherein binding domain B1 comprises:
(a) the heavy chain variable region and/or the light chain variable region of antibody 1132/1133 (SEQ ID NOs: 3 and 1); or
(b) the heavy chain variable region and/or the light chain variable region of antibody 1107/1108 (SEQ ID NOs: 15 and 13); or
(c) the heavy chain variable region and/or the light chain variable region of antibody 1150/1151 (SEQ ID NOs: 7 and 5); or
(d) the heavy chain variable region and/or the light chain variable region of antibody 1140/1135 (SEQ ID NOs: 11 and 9); or
(e) the heavy chain variable region and/or the light chain variable region of antibody ADC-1013 (SEQ ID NOs: 19 and 17); or
(f) the heavy chain variable region and/or the light chain variable region of antibody APX005 (SEQ ID NOs: 23 and 21); or
(g) the heavy chain variable region and/or the light chain variable region of antibody 21.4.1 (SEQ ID NOs: 27 and 25).

32. A bispecific polypeptide according to any one of the preceding claims wherein binding domain B1 comprises the light chain of antibody 1132/1133 (SEQ ID NO: 182) and/or the heavy chain of antibody 1132/1133 (SEQ ID NO: 181).

33. A bispecific polypeptide according to any one of the preceding claims wherein binding domain B2 binds to a tumour cell-associated antigen selected from the group consisting of:
(a) products of mutated oncogenes and tumour suppressor genes;
(b) overexpressed or aberrantly expressed cellular proteins;
(c) tumour antigens produced by oncogenic viruses;
(d) oncofetal antigens;
(e) altered cell surface glycolipids and glycoproteins;
(f) cell type-specific differentiation antigens;
(g) hypoxia-induced antigens;
(h) tumour peptides presented by MHC class I;
(i) epithelial tumour antigens;
(j) haematological tumour-associated antigens;
(k) cancer testis antigens; and
(l) melanoma antigens.

34. A bispecific polypeptide according to any one of the preceding claims wherein the tumour cell-associated antigen is selected from the group consisting of 5T4, CD20, CD19, MUC-1, carcinoembryonic antigen (CEA), CA-125, CO17-1A, EpCAM, HER2, HER3, EphA2, EphA3, DR4, DR5, FAP, OGD2, VEGFR, EGFR, NY-ESO-1, survivin, TROP2 and WT-1.

35. A bispecific polypeptide according to any one of the preceding claims wherein the tumour cell-associated antigen is an oncofetal antigen.

36. A bispecific polypeptide according to any one of the preceding claims wherein the tumour cell-associated antigen is 5T4.

37. A bispecific polypeptide according to claim 34, wherein the tumour cell-associated antigen is selected from the group consisting of CD20, EGFR, EpCAM and HER2.

38. A bispecific polypeptide according to claim 37, wherein the tumour-cell associated antigen is EpCAM.

39. A bispecific polypeptide according to any one of the preceding claims wherein the tumour cell is a solid tumour cell.

40. A bispecific polypeptide according to claim 39 wherein the solid tumour is selected from the groups consisting of renal cell carcinoma, colorectal cancer, lung cancer, prostate cancer, breast cancer, melanomas, bladder cancer, brain/CNS cancer, cervical cancer, oesophageal cancer, gastric cancer, head/neck cancer, kidney cancer, liver cancer, leukaemia, lymphomas, ovarian cancer, pancreatic cancer and sarcomas.

41. A bispecific polypeptide according to any one of the preceding claims wherein binding domain B2 binds to the tumour cell-associated antigen with a K_{D} of less than 100×10⁻⁹M, for example less than 10×10⁻⁹M or less than 5×10⁻⁹M.

42. A bispecific polypeptide according to any one of the preceding claims wherein binding domain B2 comprises one or more heavy chain CDR sequences selected from those in Table D(1) and/or wherein binding domain B2 comprises one or more light chain CDR sequences selected from those in Table D(2).

43. A bispecific polypeptide according to any one of the preceding claims, wherein binding domain B2 comprises one, two or three light chain CDR sequences from a particular row for an individual antibody reference in Table D(2), and/or one, two or three heavy chain CDR sequences from the corresponding row for the antibody with the same reference in Table D(1).

44. A bispecific polypeptide according to any one of the preceding claims wherein binding domain B2 comprises all three heavy chain CDR sequences of a particular antibody reference as shown in Table D(1), and/or all three light chain CDR sequences of an antibody reference as shown in Table D(2), or wherein binding domain B2 comprises a heavy chain VH sequence and/or a light chain VL sequence as shown in Table B.

45. A bispecific polypeptide according to any one of the preceding claims wherein binding domain B2 comprises:
(a) the three CDRs of the light chain and/or the three CDRs of the heavy chain of antibody Solitomab (SEQ ID NOs: 136, 137, and 138 and/or SEQ ID NOs: 105, 106 and 107); or
(b) the three CDRs of the light chain and/or the three CDRs of the heavy chain of antibody 005025 (SEQ ID NOs: 90, 91, and 139 and/or SEQ ID NOs: 108, 109 and 110); or
(c) the three CDRs of the light chain and/or the three CDRs of the heavy chain of antibody 005038 (SEQ ID NOs: 90, 91, and 140 and/or SEQ ID NOs: 108, 109 and 111); or
(d) the three CDRs of the light chain and/or the three CDRs of the heavy chain of antibody Adecatumumab (SEQ ID NOs: 90, 137, and 141 and/or SEQ ID NOs: 112, 113 and 114); or
(e) the three CDRs of the light chain and/or the three CDRs of the heavy chain of antibody 4D5MOCB (SEQ ID NOs: 142, 143, and 144 and/or SEQ ID NOs: 115, 116 and 117); or
(f) the three CDRs of the light chain and/or the three CDRs of the heavy chain of antibody 3-171 (SEQ ID NOs: 145, 146, and 147 and/or SEQ ID NOs: 118, 119 and 120); or
(g) the three CDRs of the light chain and/or the three CDRs of the heavy chain of antibody Trastuzumab (SEQ ID NOs: 148, 149, and 150 and/or SEQ ID NOs: 121, 122 and 123); or
(h) the three CDRs of the light chain and/or the three CDRs of the heavy chain of antibody Pertuzumab (SEQ ID NOs: 151, 149, and 152 and/or SEQ ID NOs: 124, 125 and 126); or
(i) the three CDRs of the light chain and/or the three CDRs of the heavy chain of antibody 2992/2993 (SEQ ID NOs: 153, 91, and 154 and/or SEQ ID NOs: 127, 128 and 129); or
(j) the three CDRs of the light chain and/or the three CDRs of the heavy chain of antibody Rituximab (SEQ ID NOs: 155, 156, and 157 and/or SEQ ID NOs: 130, 131 and 132); or
(k) the three CDRs of the light chain and/or the three CDRs of the heavy chain of antibody Cetuximab (SEQ ID NOs: 158, 159, and 160 and/or SEQ ID NOs: 133, 134 and 135).

46. A bispecific polypeptide according to any one of the preceding claims wherein binding domain B2 comprises:
(a) the light chain variable region and/or the heavy chain variable region of antibody Solitomab (SEQ ID NO: 29 and 31) or
(b) the light chain variable region and/or the heavy chain variable region of antibody 005025 (SEQ ID NO: 35 and 36) or
(c) the light chain variable region and/or the heavy chain variable region of antibody 005038 (SEQ ID NO: 39 and 40) or
(d) the light chain variable region and/or the heavy chain variable region of antibody Adecatumumab (SEQ ID NO: 41 and 43) or
(e) the light chain variable region and/or the heavy chain variable region of antibody 4D5MOCB (SEQ ID NO: 45 and 47) or
(f) the light chain variable region and/or the heavy chain variable region of antibody 3-17I (SEQ ID NO: 49 and 51) or
(g) the light chain variable region and/or the heavy chain variable region of antibody Trastuzumab (SEQ ID NO: 53 and 55) or
(h) the light chain variable region and/or the heavy chain variable region of antibody Pertuzumab (SEQ ID NO: 57 and 59) or
(i) the light chain variable region and/or the heavy chain variable region of antibody 2992/2993 (SEQ ID NO: 61 and 63) or
(j) the light chain variable region and/or the heavy chain variable region of antibody Rituximab (SEQ ID NO: 65 and 67) or
(k) the light chain variable region and/or the heavy chain variable region of antibody Cetuximab (SEQ ID NO: 69 and 71).

47. A bispecific polypeptide according to any one of the preceding claims wherein binding domain B1 is an IgG and binding domain B2 is an scFv.

48. A bispecific polypeptide according to any one of claims 1 to 46 wherein binding domain B1 is an scFv and binding domain B2 is an IgG.

49. A bispecific polypeptide according to any one of claims 1 to 46 wherein binding domain B1 is an IgG and binding domain B2 is a Fab.

50. A bispecific polypeptide according to any one of claims 1 to 46 wherein binding domain B1 is a Fab and binding domain B2 is an IgG.

51. A bispecific polypeptide according to any one of the preceding claims wherein:
(a) B1 comprises the three CDRs of the heavy chain and/or the three CDRs of the light chain of antibody 1132 (SEQ ID NOs: 73, 74 and 75 and/or SEQ ID NOs: 90, 91, and 92) and B2 comprises the three CDRs of the heavy chain and/or the three CDRs of the light chain of antibody Solitomab (SEQ ID NOs: 105, 106 and 107 and/or SEQ ID NOs: 136, 137, and 138); or
(b) B1 comprises the three CDRs of the heavy chain and/or the three CDRs of the light chain of antibody 1132 (SEQ ID NOs: 73, 74 and 75 and/or SEQ ID NOs: 90, 91, and 92) and B2 comprises the three CDRs of the heavy chain and/or the three CDRs of the light chain of antibody 2992 (SEQ ID NOs: 127, 128 and 129 and/or SEQ ID NOs: 153, 91, and 154); or
(c) B1 comprises the three CDRs of the heavy chain and/or the three CDRs of the light chain of antibody 1132 (SEQ ID NOs: 73, 74 and 75 and/or SEQ ID NOs: 90, 91, and 92) and B2 comprises the three CDRs of the heavy chain and/or the three CDRs of the light chain of antibody Trastuzumab (SEQ ID NOs: 121, 122 and 123 and/or SEQ ID NOs: 148, 149, and 150).

52. A bispecific polypeptide according to any one of the preceding claims wherein B1 comprises a heavy chain comprising the sequence of SEQ ID NO: 181, and a light chain comprising the sequence of SEQ ID NO: 182, and/or B2 comprises a heavy chain comprising the sequence of SEQ ID NO: 183, and a light chain comprising the sequence of SEQ ID NO: 184.

53. An isolated nucleic acid molecule encoding a bispecific polypeptide according to any one of the preceding claims, or a component polypeptide chain thereof.

54. A nucleic acid molecule according to claim 53 wherein the molecule is a cDNA molecule.

55. A nucleic acid molecule according to claim 53 or 54 encoding an antibody heavy chain or variable region thereof.

56. A nucleic acid molecule according to any one of claims 53 to 55 encoding an antibody light chain or variable region thereof.

57. A vector comprising a nucleic acid molecule according to any one of claims 53 to 56.

58. A vector according to claim 57 wherein the vector is an expression vector.

59. A recombinant host cell comprising a nucleic acid molecule according to any one of claims 52 to 55 or a vector according to claim 57 or 58.

60. A host cell according to claim 59 wherein the host cell is a bacterial cell.

61. A host cell according to claim 59 wherein the host cell is a mammalian cell.

62. A host cell according to claim 59 wherein the host cell is a human cell.

63. A method for producing bispecific polypeptide according to any one of claims 1 to 52, the method comprising culturing a host cell as defined in any of claims 59 to 62 under conditions which permit expression of the bispecific polypeptide or component polypeptide chain thereof.

64. A pharmaceutical composition comprising an effective amount of bispecific polypeptide according to any one of the claims 1 to 52 and a pharmaceutically-acceptable diluent, carrier or excipient.

65. A pharmaceutical composition according to claim 64 adapted for parenteral delivery.

66. A pharmaceutical composition according to claim 64 adapted for intravenous delivery.

67. A bispecific polypeptide according to any one of the claims 1 to 52 for use in medicine.

68. A bispecific polypeptide according to any one of the claims 1 to 52 for use in treating or preventing a neoplastic disorder in a subject.

69. A polypeptide for use according to claim 68 wherein the neoplastic disorder is associated with the formation of solid tumours within the subject's body.

70. A polypeptide for use according to claim 69 wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, leukemia, lymphomas, ovarian cancer, pancreatic cancer and sarcomas.

71. A polypeptide for use according to claim 70 wherein the solid tumour is selected from the groups consisting of renal cell carcinoma, colorectal cancer, lung cancer, prostate cancer, ovarian cancer, bladder cancer and breast cancer.

72. A polypeptide for use according to any one of claims 67 to 71 wherein the polypeptide is for use in combination with one or more additional therapeutic agents.

73. A polypeptide for use according to claim 72 wherein the one or more additional therapeutic agents is/are an immunotherapeutic agent that binds a target selected from the group consisting of PD-1/PD-L1, CTLA-4, CD137, OX40, GITR, LAG3, TIM3, CD27, VISTA and KIR.

74. Use of a bispecific polypeptide according to any one of claims 1 to 52 in the preparation of a medicament for treating or preventing a neoplastic disorder in a subject.

75. A use according to claim 74 wherein the neoplastic disorder is associated with the formation of solid tumours within the subject's body.

76. A use according to claim 75 wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, leukaemia, lymphomas, ovarian cancer, pancreatic cancer and sarcomas.

77. A use according to claim 76 wherein the solid tumour is selected from the groups consisting of renal cell carcinoma, colorectal cancer, lung cancer, prostate cancer, ovarian cancer, bladder cancer and breast cancer.

78. A use according to any one of claims 74 to 77 wherein the polypeptide is for use in combination with one or more additional therapeutic agents.

79. A polypeptide for use according to claim 78 wherein the one or more additional therapeutic agents is/are an immunotherapeutic agent that binds a target selected from the group consisting of PD-1/PD-L1, CTLA-4, CD137, OX40, GITR, LAG3, TIM3, CD27 and KIR.

80. A method for the treatment or diagnosis of a neoplastic disorder in a subject, comprising the step of administering to the subject an effective amount of a bispecific polypeptide according to any one of the claims 1 to 52.

81. A method according to claim 80 wherein the neoplastic disorder is associated with the formation of solid tumours within the subject's body.

82. A method according to claim 81 wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, leukaemia, lymphomas, ovarian cancer, pancreatic cancer and sarcomas.

83. A method according to claim 82 wherein the solid tumour is selected from the groups consisting of renal cell carcinoma, colorectal cancer, lung cancer, prostate cancer, ovarian cancer, bladder cancer and breast cancer.

84. A method according to any one of claims 80 to 83 wherein the subject is human.

85. A method according to any one of claims 80 to 84 wherein the method comprises administering the bispecific polypeptide systemically.

86. A method according to any one of claims 80 to 85 further comprising administering to the subject one or more additional therapeutic agents.

87. A method according to any one of claims 80 to 86 wherein the one or more additional therapeutic agents is/are an immunotherapeutic agent that binds a target selected from the group consisting of PD-1/PD-L1, CTLA-4, CD137, OX40, GITR, LAG3, TIM3, CD27 and KIR.

88. A bispecific polypeptide substantially as described herein with reference to the description and figures.

89. A polynucleotide substantially as described herein with reference to the description and figures.

90. A pharmaceutical composition substantially as described herein with reference to the description and figures.

91. Use of a bispecific polypeptide substantially as described herein with reference to the description and figures.

92. A method of treatment substantially as described herein with reference to the description and figures.
